SEQUENCE LISTING (2 sequences)

SEQ ID NO: 1 - DNA, 1741 bp, Homo sapiens
ctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccattcctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaaccctcggccagcaggtcccctatgccaccaagggcaaccagtgggtaggatacgacgaccaggaaagcgtcaaaagcaaggtgcagtacctgaaggataggcagctggcaggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatcctacttccccttcctaattccacagctgctcaataaagtacaagagtttaacagtgtg

SEQ ID NO: 2 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

(19) United States
(12) Patent Application Publication
(10) Pub. No.: US 2012/0040355 A1
(43) Pub. Date: Feb. 16, 2012

(54) YKL-40 AS A MARKER FOR SELECTION OF TREATMENT AND MONITORING OF A DISEASE

(76) Inventors: Julia Sidenius Johansen, Frederiksberg (DK); Stig Bojesen, Kobenhavn Ø (DK); Børge Grønne Nordestgaard, Gentofte (DK); Hans Jørgen Nielsen, Kongens Lyngby (DK); Ib Jarle Christensen, Hillerød (DK)

(21) Appl. No.: 13/063,404

(22) PCT Filed: Sep. 14, 2009
(86) PCT No.: PCT/DK09/50240

§ 371 Date: Oct. 31, 2011

(30) Foreign Application Priority Data
Sep. 15, 2008 (DK) PA 2008 01292
Sep. 15, 2008 (DK) PA 2008 01294
Oct. 22, 2008 (DK) PA 2008 01465
Oct. 24, 2008 (DK) PA 2008 01471
Jan. 22, 2009 (DK) PCT/DK2009/050014
Mar. 25, 2009 (DK) PA 2009 00414
Jul. 28, 2009 (DK) PA 2009 00900

(51) Int. Cl. C12Q 1/68; G01N 33/577

(52) U.S. Cl. 435/6.12; 435/7.94

(57) ABSTRACT

The present invention relates to a methods for selecting a treatment for a specific disease or disorder, together with a method of monitoring therapeutic treatment of a specific disease or disorder and a method of determining a prognosis for a subject before, during and after administering a treatment by determining the level of YKL-40 in a sample obtained from the subject and comparing said level with one or more reference levels of YKL-40. The reference level is typically a level obtained from healthy individuals or a level previously obtained from the same subject. The present invention further relates to a kit and a device that may be used in the methods of the present invention.

### FIELD OF INVENTION

The present invention relates to a method of selecting a treatment for a specific disease or disorder in a subject and/or monitoring the progression of the disease before, during and after administering a treatment, wherein a predetermined level of YKL-40 above a reference level indicates the need for administering a treatment. The subject may suffer from a variety of diseases or disorders. The present invention further relates to a kit and a device that may be used in the method of the present invention comprising means for measuring the level of YKL-40 in a sample; and means for comparing the measured level of YKL-40 with at least one reference level of YKL-40.

### BACKGROUND OF INVENTION

Administering a treatment for a given disease or disorder is typically based on the diagnosis of the disease and occasionally on the severity of the disease disregarding the physiology of the individual suffering from the particular disease or disorder. Likewise, the continued treatment of a disease or disorder is often according to a predetermined schedule, without paying too much attention to the individual patient.

A single marker or method that would facilitate selecting between treatments of varying efficacy and/or monitoring the progression or determining the stage of a disease or disorder prior to, during and following administration of a given treatment would greatly improve the ease with which these selection and monitoration processes occur today.

The advantages associated with choosing the best possible treatment is not limited to be of benefit for the health of the individual suffering from the specific disease or disorder; it is also of benefit to the economy of the individual and the hospital/the economy of the society at large.

Previously the “Erythrocyte sedimentation rate” (also denoted sedimentation rate) has been widely used as an indicator of the presence of inflammation. The sedimentation rate is the rate at which red blood cells precipitate in a period of 1 hour. When an inflammatory process is present, the high proportion of fibrinogen in the blood causes red blood cells to stick to each other. The sedimentation rate is increased by any cause or focus of inflammation. The basal sedimentation rate is slightly higher in women and tends to rise with age. The usefulness of the sedimentation rate in asymptomatic persons is however limited by its low sensitivity and specificity, but it has been used as a sort of sickness index, when a moderate suspicion of disease was present.

At present the biomarker C-reactive protein (CRP) has mostly taken over from the previously used sedimentation rate in initial screenings for inflammation. CRP is an indicator of acute or chronic inflammation or infection, and is therefore a test of value in medicine, reflecting the presence and intensity of inflammation, although an elevation in C-reactive protein is not the telltale diagnostic sign of any one condition. Conditions which can cause a positive response in the serum CRP level are for example rheumatoid arthritis, lupus, rheumatic fever, cancer, hearth disease, cardiovascular disease, inflammatory bowel disease, and bacterial or viral infections. However not all patients with these diseases have an elevated serum CRP level, and for these patients the serum CRP level cannot be used as a sickness-index. CRP can in some cases be used to determine disease progress or the effectiveness of treatments. Since many things can cause elevated CRP, this is not a very specific prognostic indicator.

Administering the best possible treatment for each individual patient would improve the efficacy of any treatment whether it involves administration of medicaments, surgery, or other and independent of whether the treatment given is prophylactic, curative or ameliorative. A classification of the individuals suffering from a disease or disorder according to survival prognosis would be of assistance in determining the best possible treatment, improve the effect of an administered treatment, improve the survival rate, lower relapse risks, and heighten the quality of life following the outbreak of a disease or disorder.

Monitoring the treatment administered to any individual patient depending upon the progression and/or state of their disease or disorder would be of assistance in determining the most effective immediate and follow-up treatment, and be of guidance when counseling on e.g. lifestyle changes required subsequent to the occurrence of a disease or disorder.

### SUMMARY OF INVENTION

The present invention relates to a method for determining a therapy for and/or monitoring a therapeutic treatment of a specific disease or disorder in a subject, said method comprising:
i) determining the level of YKL-40 in a sample obtained from the subject;
ii) comparing the level of YKL-40 with one or more reference levels of YKL-40, wherein the level of YKL-40 with respect to the reference levels indicates the progress and/or state of said specific disease or disorder; and
iii) deducing the progress and/or state of said specific disease or disorder by said comparison, and based thereon determining a therapy to be initiated, continued, terminated or replaced.

Thus a first aspect of the present invention relates to a method for determining a therapy for a specific disease or disorder in a subject, said method comprising:
i) determining the level of YKL-40 in a sample obtained from the subject; and
ii) comparing the level of YKL-40 with one or more reference levels of YKL-40 from the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years),

and

the 97.5^{th} percentile: In(plasma YKL-40 μg/l)=3.9+0.02×age (years);

wherein the level of YKL-40 with respect to the reference levels indicates the progress and/or state of said specific disease or disorder, and therefore the therapy to be initiated or continued.

A second aspect of the present invention relates to a method for monitoring therapeutic treatment of a specific disease or disorder in a subject, said subject being treated for the specific disease, said method comprising
i) determining the level of YKL-40 in a sample obtained from the subject;
ii) comparing the level of YKL-40 with one or more reference levels of YKL-40 from the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years),

and

the 97.5^{th} percentile: In(plasma YKL-40 μg/l)=3.9+0.02×age (years);

or
comparing the level of YKL-40 with one or more previously determined levels of YKL-40 from the same subject:
   where a level of YKL-40 in the sample being increased to at least a factor 1.10 compared to the reference level of YKL-40 indicates that the disease or disorder has evolved to a more severe stage; and
   where a level of YKL-40 in the sample being decreased to at least a factor 0.90 compared to the reference level of YKL-40 indicates that the disease or disorder has evolved to a less severe stage;
wherein the level of YKL-40 with respect to the reference levels indicates the progress and/or state of said specific disease or disorder, and therefore the degree of efficacy of the ongoing therapeutic treatment; and
iii) based thereon determining whether the therapeutic treatment of the specific disease or disorder is to be continued, terminated or replaced.

A third aspect of the present invention relates to a method for determining a prognosis for a subject suffering from a specific disease or disorder, said method comprising
i) determining the level of YKL-40 in a sample obtained from the subject;
ii) comparing said level of YKL-40 with one or more reference levels of YKL-40;
   wherein the level of YKL-40 with respect to the reference levels indicates the development or progression of said disease or disorder during or after the specific treatment regime and therefore the prognosis.

In one embodiment of the methods of the invention the one or more reference levels of YKL-40 is one or more previously determined levels of YKL-40 from the same subject. In this case a level of YKL-40 in the sample being increased to at least a factor 1.10 compared to the reference level of YKL-40 indicates that the disease or disorder has evolved to a more severe stage of the disease or disorder, and thus e.g. requires a therapy of high efficacy to be initiated and/or requires a therapy with higher efficacy than the ongoing therapy to be initiated; and

a level of YKL-40 in the sample being decreased to at least a factor 0.90 compared to the reference level of YKL-40 indicates that the disease or disorder has evolved to a less severe stage of the disease or disorder and thus e.g. requires a therapy of low efficacy to be initiated and/or requires a therapy with lower efficacy than the ongoing therapy to be initiated.

The present invention as described herein further relates to a device for determining a therapy for and/or monitoring a therapeutic treatment of a specific disease or disorder in a subject, wherein the device comprises means for measuring the level of YKL-40 in a sample; and means for comparing the measured level of YKL-40 with one or more reference levels of YKL-40. Furthermore, the present invention as described herein relates to a kit of parts comprising i) means for measuring the level of YKL-40 in a sample; ii) means for comparing the measured level of YKL-40 with one or more reference level of YKL-40; and iii) instructions on how to age adjust the reference level of YKL-40, according to the age of the subject providing the sample.

### DESCRIPTION OF DRAWINGS

FIG. 1. Plasma concentrations of YKL-40 in 2116 healthy women and 1494 healthy men according to age and sex. The participants had no known disease at the time of blood sampling in 1991-1994 and remained healthy during the 16 years follow-up period (i.e. none were dead or had developed cancer, ischaemic cardiovascular disease, liver disease, diabetes, chronic obstructive pulmonary disease, asthma, rheumatoid arthritis, inflammatory bowel disease, and pneumonia). The median plasma YKL-40 in these healthy participants was 42 μg/L (2.5%-97.5% percentile range: 14-168 μg/L; 90% percentile 92 μg/L; 95% percentile 124 μg/L). Plasma YKL-40 levels increased in both sexes with increasing age (trend test p<0.0001). Spearman's rho correlation between plasma YKL-40 and age was 0.41 (p<0.0001). There was no difference between plasma YKL-40 in women and men (Mann-Whitney U; p=0.27).

FIG. 2. Plasma concentrations of YKL-40 in a group of 929 healthy participants (463 women and 466 men), who had their first YKL-40 measurement in the blood from the 1991-1994 examination and the second YKL-40 measurement in the blood from the 2001-2003 examination. The mean increase was 0.5 μg/L/year (interquartile range −0.6-2.1 μg/L/year) in women and 0.8 μg/L/year (−0.3-2.9 μg/L/year) in men. This illustrates that plasma YKL-40 is very stable in subjects that remain healthy and a regression dilution ratio of 0.8042 was computed. There was no statistically difference between men and women.

FIG. 3A. Plasma concentrations of YKL-40 were determined in 2116 healthy women and 1494 healthy men. The participants had no known disease at the time of blood sampling in 1991-1994 and remained healthy during the 16 years follow-up period (i.e. none were dead or had develop cancer, ischaemic cardiovascular disease, liver disease, diabetes, chronic obstructive pulmonary disease, asthma, rheumatoid arthritis, inflammatory bowel disease, and pneumonia). The figure illustrates the 50% percentile plasma YKL-40 in these healthy participants (circles), the 70% percentile (defined as In(plasma YKL-40)=3.1+0.02×age (years)), the 75% percentile (defined as In(plasma YKL-40)=3.2+0.02×age (years)), the 90 percentile (defined as In(plasma YKL-40)=3.5+0.02×age (years)) and the 95% percentile (defined as In(plasma YKL-40)=3.6+0.02×age (years)) according to age. Women and men were combined.

FIG. 3B. Corresponds to FIG. 3A, with additional percentiles for plasma YKL-40: the 85% percentile (defined as In(plasma YKL-40)=3.4+0.02×age (years)), and the 97.5% percentile (defined as In(plasma YKL-40)=3.9+0.02×age (years)).

FIG. 4A. Longevity and survival of the general population according to increasing plasma concentrations of YKL-40 (divided into five gender and 10-year age percentile categories: 0-33% percentile, 34-66%, 67-90%, 91-95%, and 96-100%). Left-truncated age and follow-up time were the underlying time-scales, respectively. Follow-up started at time of blood sampling and ended at death or July 2007, whichever came first. Women and men are combined. For comparison the effect of smoking status in the same population is shown.

FIG. 4B. Absolute 10-year mortality according to plasma YKL-40 percentile categories, smoking status, gender and age. Based on 8899 participants from the Copenhagen City Hearth Study 1991-1994 examination followed for 16 years. P-values are test for log-rank trend. Plasma YKL-40 percentile categories 0-33%, 34-66%, 67-90%, 91-95%, and 96-100%, are given from left to right for each of the age groupings <50 years, 50-70 years, and >70 years.

FIGS. 4C, 4D, and 4E. Kaplan-Meier 15-year survival curves according to increasing plasma concentrations of YKL-40 (divided into three gender and 10-year age percentile categories: 0-33% percentile, 34-90%, and 91-100%) in participants with cancer, liver disease (FIG. 4C), chronic obstructive pulmonary disease, ischaemic cardiovascular disease (FIG. 4D), diabetes, and asthma (FIG. 4E). Y-axis is proportion surviving, in %, X-axis is time after blood sampling, in years. Follow-up started at time of blood sampling and ended at death or July 2007, whichever came first. The participants either had the disease at time of blood sampling or it was diagnosed during the follow-up period. Women and men are combined. Multifactorially adjusted (age, sex, smoking status) hazard ratios of death are noted on each figure (left corner, bottom). P-values are test for log-rank trend. Some participants had more than one disease. The slightly lower numbers for patients with cancer and ischaemic cardiovascular disease in Table 2 are due to unknown smoking status (8 patients with cancer patients and 4 patients with ischaemic cardiovascular disease).

FIG. 5. Individual diurnal variation in serum concentrations of YKL-40 in 16 healthy subjects.

FIG. 6. Individual variation in serum YKL-40 levels of 38 healthy subjects over a period of 3 weeks.

FIG. 7. The median serum YKL-40 level for 23 individuals over 3 weeks available in each of 4 rounds (each bar represents the median of one round for each subject).

FIG. 8. Individual serum YKL-40 levels of 30 healthy women sampled over 4 weeks and repeated 3 years later for 21 of the women.

FIG. 9. A. Individual plasma YKL-40 levels in patients with metastatic upper GI cancer (n=70) and healthy subjects (n=234). B. Individual plasma YKL-40 levels in patients with localized upper GI cancer (n=40, triangles), metastatic upper GI cancer (n=70, white circles), chronic pancreatitis (n=65, upturned triangles), and healthy subjects (n=234, gray circles). The Y-axis is a logarithmic scale.

FIG. 10. Kaplan-Meier survival curves showing the association between baseline plasma YKL-40, i.e. pretreatment plasma YKL-40 levels, and overall survival in patients with metastatic upper gastrointestinal cancer. Plasma YKL-40 levels are divided in tertiles. The P-value refers to the log-rank test for equality of strata.

FIG. 11. Box-plots showing plasma YKL-40 at baseline and during treatment with chemotherapy in patients with metastatic upper gastrointestinal cancer. The Y-axis is a logarithmic scale.

FIG. 12. Kaplan-Meier survival curves showing the association between plasma YKL-40 level after 4 weeks of chemotherapy and overall survival in patients with metastatic upper gastrointestinal cancer. Plasma YKL-40 levels are divided in tertiles. The P-value refers to the log-rank test for equality of strata.

FIG. 13. Kaplan-Meier survival curves showing the association between plasma YKL-40 level 4-6 weeks after end of radiochemotherapy and overall survival in patients with localized pancreatic cancer. Plasma YKL-40 levels are dichotomized according to an increase or decrease/no change compared to the baseline level.

FIG. 14. Kaplan-Meier survival curves showing the association between the ratios of plasma YKL-40 in samples collected 4-6 weeks after end of chemoradiotherapy in patients with locally advanced pancreatic cancer (CORGI Study) and overall survival. The ratios are calculated as YKL-40 level after 4-6 weeks of treatment over YKL-40 baseline level, i.e. pretreatment level. The upper curve is the group with low ratios, and the lower curve the group with high ratios. The P-value refers to the log-rank test for equality of strata.

FIG. 15. Kaplan-Meier survival curves showing the association between the ratios of plasma YKL-40 in samples collected 4 weeks after start of chemotherapy in patients with metastatic pancreatic cancer (GITAC Study) and overall survival. The ratios are calculated as YKL-40 level after 4 weeks of treatment over YKL-40 baseline level, i.e. pretreatment level. The upper curve is the group with low ratios, and the lower curve the group with high ratios. The P-value refers to the log-rank test for equality of strata.

FIG. 16. Kaplan-Meier survival curves showing the association between pre-treatment plasma YKL-40 levels and overall survival in patients with metastatic colorectal cancer treated with irinotecan and Cetuximab every second week. Plasma YKL-40 levels are divided in tertiles. The upper curve is the tertile with the lowest YKL-40 levels, the curve in the middle is the tertile with the medium YKL-40 levels, and the bottom curve is the tertile with the highest YKL-40 levels. The P-value refers to the log-rank test for equality of strata.

FIGS. 17A and 17B Dipstick embodiments seen from above. Dipstick support material (1.) with assay field (2.) for use with the biological sample and one control or standard field (3. in FIG. 17A) or multiple control or standard fields (4a. to 4.e. in FIG. 17B). Standards of a single (for 3.) or various (one concentration for each field in increasing or decreasing order, e.g.) YKL-40 concentrations may be applied to the control or standard fields to enable reading a positive/negative result with the stick portrayed in FIG. 17A or assessing an approximate concentration of YKL-40 in the biological sample compared to which of the control fields in FIG. 17B the sample/assay field resembles the most, post testing.

FIG. 18. Study 2. Kaplan-Meier curves showing the association between the pretreatment serum YKL-40 levels and progression free survival in patients with metastatic colorectal cancer treated with irinotecan and cetuximab. The P-value refers to the log-rank test for equality of strata. Patients are divided into tertiles according to their pretreatment serum YKL-40 levels. Patients in Group 3 have the highest serum YKL-40 levels. Serum YKL-40: Group 1: <94 μg/l; Group 2: ≧94 and ≦253 μg/l; and Group 3: >253 μg/l.

FIG. 19. Study 1. Kaplan-Meier curves showing the association between the pretreatment plasma YKL-40 levels and overall survival in patients with metastatic colorectal cancer treated with irinotecan and cetuximab (FIG. 19A). The P-value refers to the log-rank test for equality of strata. Patients are divided into tertiles according to their pretreatment plasma YKL-40 levels. Patients in Group 3 have the highest plasma YKL-40 levels. Plasma YKL-40: Group 1: <84 μg/l; Group 2: ≧84 and ≦218 μg/l; and Group 3: >218 μg/l.

Study 2. Kaplan-Meier curves showing the association between the pretreatment serum YKL-40 levels and overall survival in patients with metastatic colorectal cancer treated with irinotecan and cetuximab (FIG. 19B). The P-value refers to the log-rank test for equality of strata. Patients are divided into tertiles according to their pretreatment serum YKL-40 levels. Patients in Group 3 have the highest serum YKL-40 levels. Serum YKL-40: Group 1: <94 μg/l; Group 2: ≧94 and ≦253 μg/l; and Group 3: >253 μg/l.

FIG. 20. Study 1. Kaplan-Meier curves showing the association between the pretreatment plasma YKL-40 levels and overall survival in patients with metastatic colorectal cancer treated with irinotecan and cetuximab according to KRAS status (FIG. 20A: wild type; FIG. 20B: mutations). The P-value refers to the log-rank test for equality of strata. Patients are divided into tertiles according to their pretreatment plasma YKL-40 levels. Patients in Group 3 have the highest plasma YKL-40 levels. Plasma YKL-40: Group 1: <84 μg/l; Group 2: ≧84 and ≦218 μg/l; and Group 3: >218 μg/l.

Study 2. Kaplan-Meier curves showing the association between the pretreatment serum YKL-40 levels and overall survival in patients with metastatic colorectal cancer treated with irinotecan and cetuximab according to KRAS status (FIG. 20C: wild type; FIG. 20D: mutations). The P-value refers to the log-rank test for equality of strata. Patients are divided into tertiles according to their pretreatment serum YKL-40 levels. Patients in Group 3 have the highest serum YKL-40 levels. Serum YKL-40: Group 1: <94 μg/l; Group 2: ≧94 and ≦253 μg/l; and Group 3: >253 μg/l.

FIG. 21. Study 1. Kaplan-Meier curves showing the association between the pretreatment plasma YKL-40 levels and overall survival in patients with metastatic colorectal cancer treated with irinotecan and cetuximab according to increasing cut-off levels of plasma YKL-40 in healthy subjects (age-corrected). FIG. 21A: 90 percentile; FIG. 21B: 95 percentile; FIG. 21C: 97.5 percentile; FIG. 21D: 99 percentile; FIG. 21E: 99.5 percentile; and FIG. 21F: 99.9 percentile. The P-value refers to the log-rank test for equality of strata.

FIG. 22. Study 2. Kaplan-Meier curves showing the association between the pretreatment serum YKL-40 levels and overall survival in patients with metastatic colorectal cancer treated with irinotecan and cetuximab according to increasing cut-off levels of serum YKL-40 in healthy subjects (age-corrected). FIG. 22A: 90 percentile; FIG. 22B: 95 percentile; FIG. 22C: 97.5 percentile; FIG. 22D: 99 percentile;

FIG. 22E: 99.5 percentile; and FIG. 22F: 99.9 percentile. The P-value refers to the log-rank test for equality of strata.

FIG. 23A-B. Individual changes in YKL-40 (μg/l) in patients with metastatic colorectal cancer during treatment with cetuximab and irinotecan. The results from Study 1 are shown in A, and from Study 2 in B.

FIG. 24A-B. Individual changes in YKL-40 (ratio) (calculated/defined as the level at different time points during the treatment divided by the baseline level =pre-treatment level) in patients with metastatic colorectal cancer during treatment with cetuximab and irinotecan. The results from Study 1 are shown in A, and from Study 2 in B.

FIG. 25A-B. Kaplan-Meier survival curves of progression free survival (A) and overall survival (B) showing the association between the ratios of YKL-40 in blood samples collected 2-3 months after start of cetuximab treatment and compared to baseline YKL-40 levels in patients with metastatic colorectal cancer (the ratio is calculated/defined as the level of YKL-40 after 2-3 months of treatment divided by the baseline level =pre-treatment level). Low ratio (≦1) reflects a decrease in YKL-40 at 2-3 months compared to pre-treatment. High ratio (>1) reflects an increase in YKL-40 at 2-3 months compared to pre-treatment. The P-value refers to the log-rank test for equality of strata. The patients are dichotomized in two groups with high or low ratio.

### DETAILED DESCRIPTION OF THE INVENTION

The present inventors have surprisingly found that the YKL-40 level can be used as a biomarker for determining a therapy for and/or monitoring a therapeutic treatment of a specific disease or disorder in a subject, said based on the classification of the severity of a specific disease or disorder and/or based on the determined prognosis for the subject, by comparison with one or more reference levels of YKL-40. The present inventors have furthermore found that the YKL-40 level can be used as a marker for keeping track of the development of a disease or disorder, i.e. whether the disease or disorder evolve towards a more or a less severe stage of a diseases or disorder, hereby repeatedly and/or continuously classifying the severity of a disease or disorder over time and thus allowing for the determination of whether to continue the ongoing treatment, replace the treatment with one of higher or lower efficacy or simply alter the administration of the ongoing treatment as well as whether it is prudent to terminate the ongoing treatment. This is especially interesting and feasible when a YKL-40 measurement in a subject is compared to one or more reference levels which are previously obtained measurement from the same subject. Accordingly, by the methods according to the present invention the YKL-40 level can be used not only to determine which treatment to administer, but also to determine which treatment to continue with as determined by monitoration of the therapeutic treatment administered.

The following definitions are provided to simplify discussion of the invention. They should not, therefore, be construed as limiting the invention, which is defined in scope by the appended claims and the specification in its entirety.

The terms “a specific disease or disorder” “a specific disease” or “a specific disorder”, as used herein, are intended to mean a disease or disorder that is known, i.e. being diagnosed prior to the administration of the best possible therapy and/or treatment. The subject may in fact be undergoing a therapy or treatment but this therapy deemed suboptimal as the severity of the disease/the state of the disease or disorder is unknown at the time of administration of the initial therapy or therapeutic treatment.

An example of a widely used general biomarker for inflammation is serum C-reactive protein (CRP). CRP is often used in connection with an initial screening, and is for instance used as a rough indicator of risk of heart disease, cardiovascular disease, bacterial infections, viral infections etc. However, some patients with diseases or disorders will not have an increase in the serum CRP level, and the CRP level can therefore not be used as a sickness index for all patients with these diseases.

Before CRP became widely used and well-known, the Erythrocyte Sedimentation Rate (often referred to as Sedimentation Rate) was used in an initial screening as a non-specific measure of inflammation, i.e. as a sickness index.

The methods according to the present invention provide a new biomarker in the form of the YKL-40 level and provide a method of classifying the severity of non-specific disease or disorder. It has been found that YKL-40 can be used not only to determine the severity of a non-specific disease or disorder, but also to classify whether a disease or disorder in a subject evolves towards a more or a less severe state of the disease or disorder. The present inventors have found the YKL-40 to be a more broadly applicable biomarker than serum CRP.

Patients with the same disease can have marked differences in the disease severity (i.e. different grades of how serious the disease is). The terms “severe stage”, “severity”, “less severe” and “more severe”, as used herein, are intended to mean a graduation of severity according to for example prognosis for being cured, prognosis for survival, or according to different predetermined stages of diseases. Such stages may be according to various symptoms, and/or traditionally measureable levels of biomarkers, physical functions etc. When focusing on the development of a disease in one and same subject, then a more severe stage refers to a worsening of the disease, whereas a less severe stage than previously determined refers to a bettering of the disease, e.g. due to a satisfactory treatment regime. As the prognosis of a patient may be independent of a classical staging of the disease in question, the terms “a more severe stage” and “a less severe stage”, as used herein, is also intended to mean a worsening or an improvement of the prognosis of the patient, respectively. For patients suffering from a gastrointestinal cancer disease the prognosis is typically a prognosis relating to expected time before disease progression, or time before death. Accordingly, a worsening of the prognosis typically corresponds to a shorter progression free interval and/or a shorter survival period.

The terms “determining a therapy and/or therapeutic treatment”, “determining a therapy” and “determining a therapeutic treatment” cover in principle any treatment that a person skilled in the art would administer to a subject for which the YKL-40 level has been determined and compared to that of one or more reference levels. Preferably, the terms cover the most optimal therapy and/or treatment. Hereby is meant the treatment that is best suited for the individual patient in terms of any of the following: ameliorating discomfort, alleviating symptoms, curing the disease, providing the best possible quality of life and so forth for the subject. The terms “best possible” most optimal as so forth in regards to a therapy and/or therapeutic treatment are used interchangeably herein.

The therapies and or therapeutic treatments to be administered, continued, terminated, altered or replaced may be any kind of therapy such as, but not limited to the administration of medicaments, surgery, and may be prophylactic, curative or ameliorative.

A therapy and/or therapeutic treatment may be initiated if none is ongoing, or may be continued if it is already taking place. A therapy and/or therapeutic treatment may be terminated if it is found unsuitable or if it requires replacing by an alternative method of therapy and or therapeutic treatment. By altering a treatment is understood that the treatment is changed for example the dosage is increased or decreased, the concentrations of the drugs are increased or decreased, the administration/dosage regiment is increased or decreased and so on.

Accordingly, the present invention relates to a method for determining a therapy for and/or monitoring a therapeutic treatment of a specific disease or disorder in a subject, said method comprising:
i) determining the level of YKL-40 in a sample obtained from the subject;
ii) comparing the level of YKL-40 with one or more reference levels of YKL-40, wherein the level of YKL-40 with respect to the reference levels indicates the progress and/or state of said specific disease or disorder; and
iii) deducing the progress and/or state of said specific disease or disorder by said comparison, and based thereon determining a therapy to be initiated, continued, terminated or replaced.

A first aspect of the present invention relates to a method for determining a therapy for a specific disease or disorder in a subject, said method comprising:
i) determining the level of YKL-40 in a sample obtained from the subject; and
ii) comparing the level of YKL-40 with one or more reference levels of YKL-40;
   wherein the level of YKL-40 with respect to the reference levels indicates the progress and/or state of said specific disease or disorder, and therefore the therapy to be initiated or continued.

A preferred embodiment of the first aspect of the present invention relates to a method for determining a therapy for a specific disease or disorder in a subject, said method comprising:
i) determining the level of YKL-40 in a sample obtained from the subject; and
ii) comparing the level of YKL-40 with one or more reference levels of YKL-40 from the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years),

and

the 97.5^{th} percentile: In(plasma YKL-40 μg/l)=3.9+0.02×age (years);

wherein the level of YKL-40 with respect to the reference levels indicates the progress and/or state of said specific disease or disorder, and therefore the therapy to be initiated or continued.

A second aspect of the present invention relates to a method for monitoring therapeutic treatment of a specific disease or disorder in a subject, said subject being treated for the specific disease, said method comprising
i) determining the level of YKL-40 in a sample obtained from the subject;
ii) comparing the level of YKL-40 with one or more reference levels of YKL-40;
   wherein the level of YKL-40 with respect to the reference levels indicates the progress and/or state of said specific disease or disorder, and therefore the degree of efficacy of the ongoing therapeutic treatment; and
iii) based thereon determining whether the therapeutic treatment of the specific disease or disorder is to be continued, terminated or replaced.

A preferred embodiment of the second aspect of the present invention relates to a method for monitoring therapeutic treatment of a specific disease or disorder in a subject, said subject being treated for the specific disease, said method comprising
i) determining the level of YKL-40 in a sample obtained from the subject;
ii) comparing the level of YKL-40 with one or more reference levels of YKL-40 from the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years),

and

the 97.5^{th} percentile: In(plasma YKL-40 μg/l)=3.9+0.02×age (years);

or
comparing the level of YKL-40 with one or more previously determined levels of YKL-40 from the same subject:
   where a level of YKL-40 in the sample being increased to at least a factor 1.10 compared to the reference level of YKL-40 indicates that the disease or disorder has evolved to a more severe stage; and
   where a level of YKL-40 in the sample being decreased to at least a factor 0.90 compared to the reference level of YKL-40 indicates that the disease or disorder has evolved to a less severe stage;
wherein the level of YKL-40 with respect to the reference levels indicates the progress and/or state of said specific disease or disorder, and therefore the degree of efficacy of the ongoing therapeutic treatment; and
iii) based thereon determining whether the therapeutic treatment of the specific disease or disorder is to be continued, terminated or replaced.

A more specific embodiment of the methods of the present invention relates to a method for determining a therapy for and/or monitoring a therapeutic treatment of a specific disease or disorder in a subject, said method comprising
i) determining the level of YKL-40 in a sample obtained from the subject;
ii) comparing the level of YKL-40 with one or more reference levels of YKL-40, said reference levels being one or more previously determined levels of YKL-40 from the same subject wherein the level of YKL-40 with respect to the reference levels indicates the progress and/or state of said specific disease or disorder; and
iii) deducing the progress and/or state of said specific disease or disorder by said comparison, and based thereon determining a therapy to be initiated, continued, terminated or replaced,
   wherein a level of YKL-40 in the sample being increased to at least a factor 1.10 compared to the reference level of YKL-40 indicates that the disease or disorder has evolved to a more severe stage, and thus e.g. requires a therapy of high efficacy to be initiated and/or requires a therapy with higher efficacy than the ongoing therapy to be initiated; and
   wherein a level of YKL-40 in the sample being decreased to at least a factor 0.90 compared to the reference level of YKL-40 indicates that the disease or disorder has evolved to a less severe stage, and thus e.g. requires a therapy of low efficacy to be initiated and/or requires a therapy with lower efficacy than the ongoing therapy to be initiated.

An even more specific embodiment of the methods of the invention relates to a method for determining a therapy for a specific disease or disorder in a subject, said method comprising:
i) determining the level of YKL-40 in a sample obtained from the subject;
ii) comparing the level of YKL-40 with one or more reference levels of YKL-40, said reference levels being one or more previously determined levels of YKL-40 from the same subject wherein the level of YKL-40 with respect to the reference levels indicates the progress and/or state of said specific disease or disorder; and
iii) deducing the progress of the specific disease or disorder toward one of these predetermined stages, wherein the level of YKL-40 with respect to the reference levels indicates the progress of said specific disease or disorder, and therefore the therapy to be initiated or continued,
   wherein a level of YKL-40 in the sample being increased to at least a factor 1.10 compared to the reference level of YKL-40 indicates that the disease or disorder has evolved to a more severe stage, and thus requires a therapy of higher efficacy to be initiated; and
   wherein a level of YKL-40 in the sample being decreased to at least a factor 0.90 compared to the reference level of YKL-40 indicates that the disease or disorder has evolved to a less severe stage, and thus requires a therapy of lower efficacy to be initiated.

A third aspect of the present invention relates to a method for determining a prognosis for a subject suffering from a specific disease or disorder, said method comprising
i) determining the level of YKL-40 in a sample obtained from the subject;
ii) comparing said level of YKL-40 with one or more reference levels of YKL-40;
   wherein the level of YKL-40 with respect to the reference levels indicates the development or progression of said disease or disorder during or after the specific treatment regime and therefore the prognosis.

In a preferred embodiment of the methods of the invention, the classification of severity is performed according to prognosis of survival. In this embodiment a more severe stage corresponds to a worsening of the prognosis, and likewise, a less severe stage corresponds to bettering of the prognosis. Accordingly, when the YKL-40 level is increased it may indicate that the prognosis for the subject has worsened, and when the YKL-40 level is decreased and/or equal to the previous level it may indicate that the prognosis for the subject has become better.

A bettering of the prognosis is preferably indicated by a ratio of ≦1, i.e. that the measured YKL-40 level is below or equal to the one or more previous levels, a ratio of ≦1 also corresponds to a factor of 1, e.g. a decrease to a factor of 0.90, see herein under “reference levels” for the concept of factor. The lower ratio or factor the greater the indication that the subject has got a better prognosis, such as e.g. due to a response to a given treatment. Likewise, that the prognosis has worsened, such as e.g. due to a non-responsiveness to a treatment, is indicated by a ratio of >1, i.e. that the measured YKL-40 level is above the one or more previous levels, a ratio of >1 corresponds to a factor of >1, e.g. an increase to a factor of 1.10, see herein under “reference levels”. The higher the ratio or factor the worse is the prognosis. The increase or decrease to a higher or lower factor respectively, as described in the section “reference levels”, applies mutatis mutandis for this aspect of the invention as well.

In preferred embodiments of the second and third aspects of the invention the determination of the YKL-40 level in step i) is performed after initiation of the treatment in question. Specifically, the determination in step i) may be performed after at least 2 weeks of treatment, preferably after at least 4 weeks of treatment, or at least 6 weeks of treatment. The YKL-40 level may be determined continuously through the treatment period, such as e.g. every 2 weeks or every 1 months, as appropriate for the treatment regime in question. The determination in step i) may furthermore be performed after end of treatment, and for example regularly thereafter in a follow-up period. Follow-up measurements could for example be made every 1 month, every 2 months or every 3 months. Alternatively, or additionally, the determination in step i) is performed after end of treatment, such as at least 2 weeks after end treatment, preferably at least 4 weeks after end treatment, or at least 6 weeks after end treatment.

The methods of the present invention may furthermore be used to monitor a subject after end of treatment. Depending on the specific disease or disorder, it may be relevant to continuously monitor the subject in a follow-up period, which may be e.g. 1 year or as long as 5 to 10 years after end of treatment. By determining the YKL-40 level in the follow-up period it is possible to diagnose a re-lapse, determine the prognosis, or initiate a new or repeat a treatment. Hereby enabling the best possible treatment of the subject.

The methods according to the present invention are relevant for classifying the severity of any disease or disorder for determining the best possible treatment hereof. Said diseases or disorders may for instance be any disease of disorder for which the YKL-40 level is increased. The disease or disorder may have been diagnosed prior to, during or after the measurement of the previously determined YKL-40 levels; in a preferred embodiment, the disease or disorder is a previously diagnosed disease or disorder.

It is further an object of the present invention to provide a method for monitoring the health state of an individual suffering from any one or more diseases or disorders for determining the best possible treatment hereof in relation to a prognosis of their survival, said method comprising: measuring the level of YKL-40 in a biological sample from said individual; and comparing the measured level to a reference level of YKL-40.

It has been found that the serum or plasma YKL-40 level in an individual is stable over long time, and independent of diurnal and weekly changes; it has furthermore been found that the level is independent of at least 20 minutes of exercise. Accordingly, one measurement of the serum or plasma YKL-40 level in an individual can be used in the methods according to the invention. Preferably, the sample may be obtained from a subject that for example have abstained from heavy alcohol consumption the previous day and that for example do not have evident symptoms of e.g. bacterial infections. If necessary a second or further sample may be obtained at a later time point (e.g. after 2 weeks) to confirm the results of the first determined level of YKL-40.

It is to be emphasised that increased levels of YKL-40, such as e.g. in plasma or serum, can reflect several and diverse types of diseases and disorders, and that such increased levels of YKL-40 is not generally seen in healthy subjects. Therefore the YKL-level can be used as a sickness index according to the present invention.

The methods according to the present invention can be used to classify the severity of diseases that also may be identified and/or classified by CRP, but can furthermore be used to classify diseases that will not give a response in the CRP level. Accordingly, in one embodiment of the present invention, the specific disease or disorder is one or more diseases or disorders or a group of diseases or disorders that do not provide an elevated C-reactive protein level.

The term “ameliorate”, as used herein, is intended to mean to improve or make better; in association with a disease state a lessening in the severity or progression of a disease state, including remission or cure thereof, alternatively the perceived lessening of severity such as lessening of associated pain.

The term “antibody”, as used herein, is intended to mean Immunoglobulin molecules and active portions or fragments of immunoglobulin molecules such as Fab and F(ab′).sub.2 which are capable of binding an epitopic determinant of the YKL-40 protein. Antibodies are for example intact immunoglobulin molecules or fragments thereof retaining the immunologic activity. The term “antigen”, as used herein, is intended to mean an immunogenic full-length or fragment of an YKL-40 molecule.

The term “biological sample”, as used herein, is intended to mean a sample obtained from a subject or individual. The term “biomarker”, as used herein, is intended to mean a molecular indicator of a specific biological property, such as a pathological or physiological state. The terms “disease” and/or “disorder”, as used herein, is intended to mean an illness, injury, or disorder in a subject or individual. A disorder is often an illness or injury of a congenital type. The terms “subject” and/or “individual”, as used herein, is intended to mean a single member of a species, herein preferably a mammalian species. The term “mammal”, as used herein, is intended to include both humans and non-humans. The term “patient” as used herein, is intended to mean any individual suffering from a disease or disorder.

The term “hnRNA”, as used herein, means heteronuclear RNA. The term “mAb”, as used herein, means monoclonal antibody. The term “mRNA”, as used herein, means messenger RNA. The term “RNA”, as used herein, means any type of RNA originating alternatively isolated from nature or synthesized. The term “substantially pure”, as used herein to describe YKL-40, refers to the substantially intact molecule which is essentially free of other molecules with which YKL-40 may be found in nature.

YKL-40

YKL-40 is named based on its three N-terminal amino acids Tyrosine (Y), Lysine (K) and Leucine (L) and its molecular mass of about 40 kDa (Johansen et al. 1992). The complete amino acid (SEQ ID NO: 2) and coding sequence (SEQ ID NO: 1) of human YKL-40 is found in GenBank under Accession number: M80927. Human YKL-40 contains a single polypeptide chain of 383 amino acids and is a phylogenetically highly conserved heparin- and chitin-binding plasma glycoprotein. The sequence identity between human YKL-40 and homologs from several other mammals is: pig (84% sequence identity), cow (83%), goat (83%), sheep (83%), guinea pig, rat (80%), and mouse (73%). YKL-40 is a member of “mammalian chitinase-like proteins”, but has no chitinase activity. YKL-40 expression in vitro is absent in normal human monocytes but strongly induced during late stages of macrophage differentiation by activated monocytes and neutrophils, by vascular smooth muscle cells, cancer cells and arthritic chondrocytes. In vivo YKL-40 mRNA and protein are expressed by a subpopulation of macrophages in tissues with inflammation such as atherosclerotic plaques, arthritic vessels of individuals with giant cell arthritis, inflamed synovial membranes, sarcoid lesions, and by peritumoral macrophages.

The molecular processes governing the induction of YKL-40 and its precise functions are unknown. YKL-40 is a secreted protein suggesting that its sites of actions are most likely to be extracellular; however, specific cell-surface or soluble receptors for YKL-40 have not yet been identified. YKL-40 is a growth factor for fibroblasts and chondrocytes, acts synergistically with IGF-1, is regulated by TNF and IL-6, and requires sustained activation of NF-kappaB (Recklies et al., 2002, Ling et al., 2004, Recklies et al., 2005) YKL-40 treatment of fibroblasts can counteract the inflammatory response to TNF and IL-1 by phosphorylation of AKT, thereby attenuating ASK1 mediated signaling pathways. This leads to decreased levels of metalloproteinase and IL-8 expression (Recklies et al., 2002, Ling et al., 2004, Recklies et al., 2005). Furthermore, YKL-40 binds to collagen types I, II and III and modulates the rate of type I collagen fibril formation (Bigg et al., 2006) These observations suggest that YKL-40 may play a protective role in inflammatory environments, limiting degradation of the extracellular matrix and thereby controlling tissue remodeling. YKL-40 also acts as a chemo-attractant for endothelial cells, stimulates their migration and promotes migration and adhesion of vascular smooth muscle cells (Millis et al., 1986, Nishikawa et al., 2003; Shackelton et al., 1995) suggesting a role in angiogenesis. YKL-40 is also a growth factor for fibroblasts and has an anti-catabolic effect preserving extracellular matrix during tissue remodeling (De Ceunicnck et al., 2001, Recklies et al., 2002, Ling et al., 2004, Recklies et al., 2005). In addition, macrophages in atherosclerotic plaques express YKL-40 mRNA, particularly macrophages that have infiltrated deeper in the lesion, and the highest YKL-40 expression is found in macrophages in the early lesion of atherosclerosis (Boot et al., 1999). Furthermore YKL-40 can be regarded as an acute phase protein, since its plasma or serum concentration is increased in several inflammatory diseases.

Cellular receptors mediating the biological effects of YKL-40 are not known, but the activation of cytoplasmic signal-transduction pathways suggests that YKL-40 interacts with signaling components on the cell membrane.

It is an object of the present invention to detect any transcriptional product of the YKL-40 gene. A transcriptional product of the gene may thus be hnRNA, mRNA, full length protein, fragmented protein, or peptides of the YKL-40 protein. It is understood that one or more proteins, RNA transcripts, fragments and/or peptides may be detected simultaneously. It is furthermore an aspect of the present invention to detect transcriptional products by any means available such as by immunoassays such as antibody detection of the YKL-40 protein, fragments or peptides hereof, as well as by detection by PCR based assays such as detection of RNA by RT-PCR.

Detection of YKL-40

Peptides and polynucleotides of the invention include functional derivatives of YKL-40, YKL-40 peptides and nucleotides encoding therefore. By “functional derivative” is meant the “fragments,” “variants,” “analogs,” or “chemical derivatives” of a molecule. A “fragment” of a molecule, such as any of the DNA sequences of the present invention, includes any nucleotide subset of the molecule. A “variant” of such molecule refers to a naturally occurring molecule substantially similar to either the entire molecule, or a fragment thereof. An “analog” of a molecule refers to a non-natural molecule substantially similar to either the entire molecule or a fragment thereof.

A molecule is said to be “substantially similar” to another molecule if the sequence of amino acids in both molecules is substantially the same. Substantially similar amino acid molecules will possess a similar biological activity. Thus, provided that two molecules possess a similar activity, they are considered variants as that term is used herein even if one of the molecules contains additional amino acid residues not found in the other, or if the sequence of amino acid residues is not identical.

Further, a molecule is said to be a “chemical derivative” of another molecule when it contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th Ed., Mack Publishing Co., Easton, Pa., 1980.

Minor modifications of the YKL-40 primary amino acid sequence may result in proteins and peptides that have substantially similar activity as compared to the YKL-40 peptides described herein. Such modifications may be deliberate, as by site-directed mutagenesis, or may be spontaneous. All of the peptides produced by these modifications are included herein as long as the biological activity of YKL-40 still exists. Further, deletion of one or more amino acids can also result in a modification of the structure of the resultant molecule without significantly altering its biological activity. This can lead to the development of a smaller active molecule which would have broader utility. For example, one can remove amino or carboxy terminal amino acids which may not be required for the enzyme to exert the desired catalytic or antigenic activity.

Either polyclonal or monoclonal antibodies may be used in the immunoassays and therapeutic methods of the invention described below. Some anti-YKL-40 antibodies are available commercially or may alternatively be raised as herein described or known in the art. Polyclonal antibodies may be raised by multiple subcutaneous or intramuscular injections of substantially pure YKL-40 or antigenic YKL-40 peptides into a suitable non-human mammal. The antigenicity of YKL-40 peptides can be determined by conventional techniques to determine the magnitude of the antibody response of an animal which has been immunized with the peptide. Generally, the YKL-40 peptides which are used to raise the anti-YKL-40 antibodies should generally be those which induce production of high titers of antibody with relatively high affinity for YKL-40. In one embodiment of the invention the YKL-40 level is determined by use of a dipstick.

If desired, the immunizing peptide may be coupled to a carrier protein by conjugation using techniques which are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit). Because YKL-40 may be conserved among mammalian species, use of a carrier protein to enhance the immunogenicity of YKL-40 proteins is preferred.

The antibodies are then obtained from blood samples taken from the mammal. The techniques used to develop polyclonal antibodies are known in the art see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies, see, for example, Coligan, et al., Unit 9, Current Protocols in Immunology, Wiley Interscience, 1991).

Preferably, however, the YKL-40 antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′).sub.2, which are capable of binding an epitopic determinant. Also, in this context, the term “mAb's of the invention” refers to monoclonal antibodies with specificity for YKL-40.

The general method used for production of hybridomas secreting mAbs is well known (Kohler and Milstein, 1975). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines.

Confirmation of YKL-40 specificity among mAb's can be accomplished using relatively routine screening techniques (such as the enzyme-linked immunosorbent assay, or “ELISA”) to determine the elementary reaction pattern of the mAb of interest. It is also possible to evaluate an mAb to determine whether it has the same specificity as a mAb of the invention without undue experimentation by determining whether the mAb being tested prevents a mAb of the invention from binding to YKL-40 isolated as described above, if the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to pre-incubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

Immunoassay Procedures

The immunoassay procedure used must be quantitative so that levels of YKL-40 in an individual with disease may be distinguished from normal levels which may be present in healthy humans and/or background levels measured in the individual. Competitive and sandwich assays on a solid phase using detectible labels (direct or indirect) are, therefore, preferred. The label will provide a detectible signal indicative of binding of antibody to the YKL-40 antigen. The antibody or antigen may be labeled with any label known in the art to provide a detectible signal, including radioisotopes, enzymes, fluorescent molecules, chemiluminescent molecules, bioluminescent molecules and colloidal gold. Of the known assay procedures, radioimmunoassay (RIA) or enzyme-linked immunoassay (ELISA) are most preferred for its sensitivity. A radioisotope will, therefore, be the preferred label.

Accordingly, in a specific embodiment of the method according to the present invention the YKL-40 level is determined using an immunoassay. In one version of this embodiment the immunoassay is a competitive immunoassay.

In one embodiment of the invention, the immunoassay uses a monoclonal antibody to measure YKL-40. In an alternative embodiment of the invention the immunoassay uses a polyclonal antibody to measure YKL-40.

When a method of the present invention utilizes an immunoassay, then a detectable label selected from the group consisting of radioisotopes, enzymes, fluorescent molecules, chemiluminescent molecules, bioluminescent molecules and colloidal metals, may be used to measure YKL-40.

Examples of metallic ions which can be directly bound to an antibody, or indirectly bound to the YKL-40 antigen are well-known to those of ordinary skill in the art and include .sup.125 I, .sup.111 In, .sup.97 Ru, .sup.67 Ga, .sup.68 Ga, .sup.72 As, .sup.89 Zr, .sup.90 Y and .sup.201 TI. Preferred for its ease of attachment without compromise of antigen binding specificity is .sup.125 I (sodium salt, Amersham, United Kingdom). Labeling of YKL-40 with .sup.125 I may be performed according to the method described in Salacinski, et al. (1981). Iodogen for use to provide the .sup.125 I label (1,3,4,6-tetrachloro-3.alpha., 6.alpha.-diphenyl glycoluril) is commercially available from Pierce and Warriner, Chester, England.

In a specific preferred embodiment of the invention plasma levels of YKL-40 can be determined in duplicates by a two-site, sandwich-type enzyme-linked immunosorbent assay (ELISA) (such as e.g. the commercial Quidel, California, USA) (Harvey et al. 1998), using streptavidin-coated microplate wells, a biotinylated-Fab monoclonal capture antibody, and an alkaline phosphatase-labeled polyclonal detection antibody. When Quidel was used the recovery of the ELISA was 102% and the detection limit 10 pg/L. Sensitivity in this context is defined as the detectible mass equivalent to twice the standard deviation of the zero binding values. The standard curve will generally be linear between 20 and 300 μg/l. The intra-assay coefficients of variations were 5% (at 40 μg/L), 4% (at 104 μg/L), and 4% (at 155 μg/L). The inter-assay coefficient of variation was <6%.

In another embodiment of the invention a radioimmunoassay is used, wherein standards or samples are incubated with a substantially equal volume of YKL-40 antiserum and of YKL-40 tracer. Standards and samples are generally assayed in duplicate. The sensitivity (detection limit) of the assay of the invention is about 10 μg/l. Sensitivity in this context is defined as the detectible mass equivalent to twice the standard deviation of the zero binding values. The standard curve will generally be linear between 20 and 100 μg/l. The intra- and interassay coefficients of variance for the assay described in the following examples are <6.5% and <12%, respectively.

It will be appreciated by those skilled in the art that, although not necessarily as sensitive as an RIA, assay procedures using labels other than radioisotopes have certain advantages and may, therefore, be employed as alternatives to a RIA format. For example, an enzyme-linked immunosorbent assay (ELISA) may be readily automated using an ELISA microtiter plate reader and reagents which are readily available in many research and clinical laboratories. Fluorescent, chemiluminescent and bioluminescent labels have the advantage of being visually detectible, though they are not as useful as radioisotopes to quantify the amount of antigen bound by antibody in the assay.

PCR Based Assays

Further, it will be appreciated by those of skill in the art that means other than immunoassays may be employed to detect and quantify the presence of YKL-40 in a biological sample. For example, a polynucleotide encoding YKL-40 may be detected using quantitative polymerase chain reaction (PCR) protocols known in the art. Accordingly, in one embodiment of the method according to the present invention the YKL-40 level is determined in a PCR based assay. The preferred method for performance of quantitative PCR is a competitive PCR technique performed using a competitor template containing an induced mutation of one or more base pairs which results in the competitor differing in sequence or size from the target YKL-40 gene template. One of the primers is biotinylated or, preferably, aminated so that one strand (usually the antisense strand) of the resulting PCR product can be immobilized via an amino-carboxyl, amino--amino, biotin-streptavidin or other suitably tight bond to a solid phase support which has been tightly bound to an appropriate reactant. Most preferably, the bonds between the PCR product, solid phase support and reactant will be covalent ones, thus reliably rendering the bonds resistant to uncoupling under denaturing conditions.

Once the aminated or biotinylated strands of the PCR products are immobilized, the unbound complementary strands are separated in an alkaline denaturing wash and removed from the reaction environment. Sequence-specific oligonucleotides (“SSO's”) corresponding to the target and competitor nucleic acids are labelled with a detection tag. The SSO's are then hybridized to the antisense strands in absence of competition from the removed unbound sense strands. Appropriate assay reagents are added and the degree of hybridization is measured by ELISA measurement means appropriate to the detection tag and solid phase support means used, preferably an ELISA microplate reader. The measured values are compared to derive target nucleic acid content, using a standard curve separately derived from PCR reactions amplifying templates including target and competitor templates. This method is advantageous in that it is quantitative, does not depend upon the number of PCR cycles, and is not influenced by competition between the SSO probe and the complementary strand in the PCR product.

Alternatively, part of the polymerization step and the entire hybridization step can be performed on a solid phase support. In this method, it is a nucleotide polymerization primer (preferably an oligonucleotide) which is captured onto a solid phase support rather than a strand of the PCR products. Target and competitor nucleic acid PCR products are then added in solution to the solid phase support and a polymerization step is performed. The unbound sense strands of the polymerization product are removed under the denaturing conditions described above.

A target to competitor nucleic acid ratio can be determined by detection of labeled oligonucleotide SSO probes using appropriate measurement means (preferably ELISA readers) and standard curve as described supra. The efficiency of this method can be so great that a chain reaction in the polymerization step may be unnecessary, thus shortening the time needed to perform the method. The accuracy of the method is also enhanced because the final polymerization products do not have to be transferred from a reaction tube to a solid phase support for hybridization, thus limiting the potential for their loss or damage. If necessary for a particular sample, however, the PCR may be used to amplify the target and competitor nucleic acids in a separate reaction tube, followed by a final polymerization performed on the solid phase support.

Molecules capable of providing different, detectible signals indicative of the formation of bound PCR products known to those skilled in the art (such as labeled nucleotide chromophores which will form different colors indicative of the formation of target and competitor PCR products) can be added to the reaction solution during the last few cycles of the reaction. The ratio between the target and competitor nucleic acids can also be determined by ELISA or other appropriate measurement means and reagents reactive with detection tags coupled to the 3′ end of the immobilized hybridization primers. This method may also be adapted to detect whether a particular gene is present in the sample (without quantifying it) by performing a conventional noncompetitive PCR protocol.

Those of ordinary skill in the art will know, or may readily ascertain, how to select suitable primers for use in the above methods. For further details regarding the above-described techniques, reference may be made to the disclosures in Kohsaka, et al., Nuc. Acids Res., 21:3469-3472, 1993; Bunn, et al., U.S. Pat. No. 5,213,961; and to Innis, et al., PCR Protocols: A Guide to Methods and Applications, Acad. Press, 1990, the disclosures of which are incorporated herein solely for purposes of illustrating the state of the art regarding quantitative PCR protocols.

Reference Levels

Whether the YKL-40 level of a given subject is increased or not may be asserted by comparing a determined value with that of a reference level. The reference level may be one or more reference levels that for instance each reflects an increased severity of a specific disease or disorder, or the reference level may for instance be one or more reference levels obtained by previous measurements of samples from the same subject.

Previously, YKL-40 levels have been reported for e.g. various diseases or from healthy individuals, hereby giving an indication of the normal level. However, such previously reported “normal” YKL-40 levels from healthy individuals where not supported by a follow-up over time investigating whether the “healthy individuals” remained healthy over time. Accordingly, previously reported YKL-40 levels therefore included individuals who at the time of sampling potentially had unidentified diseases, and the reported YKL-40 levels therefore did not represent a true “normal level”. Such previously reported YKL-40 levels obtained from e.g. healthy individuals have also been reported as e.g. average levels without considering the effect of age.

As can be seen from the examples included in the present invention, the present inventors have identified a way to express a true “normal level”. This normal level has been identified on the basis of a large population of healthy individuals, and the individuals have been followed over time to confirm whether they were true “healthy individuals”. Individuals who did not continue to be healthy, e.g. who developed cancer, was removed from the normal data. The inventors have surprisingly found that the identified “normal level” can be used to classify the severity of diseases or disorders, e.g. a non-specific disease or disorder, in a subject in accordance with the methods of the present invention. The present inventors have furthermore found that age has a great influence on the YKL-40 level, and that this is to be considered when utilizing the methods of the present invention.

A reference level for YKL-40 can be expressed in various ways; traditionally reference levels may be from a group of healthy individuals of various ages. The present inventors have investigated the influence of age on the YKL-40 level and found that a measured YKL-40 level preferably is compared with age specific group.

An age specific group of individuals may comprise individuals that are all born within the same year or decade or any other groupings such as groups comprising individuals that are of 0 to 10 years of age, 10 to 20 years of age, 20 to 30 years of age, 30 to 40 years of age, 40 to 50 years of age, 50 to 60 years of age, 60 to 70 years of age, 70 to 80 years of age, 80 to 90 years of age, 90 to 100 years of age, and so on. The intervals may span 2 years of age difference, 3, 4, or 5 years of age difference, 6, 7, 8, 9, 10 years of age difference (as written), 12, 15, 20 or more years of age difference. The intervals may furthermore be open ended e.g. the individuals are all above the age of 20, 30, 40, 50, 60 or other.

The present inventors have found that there is no statistically difference between the plasma YKL-40 level in men and in women (see example 1 herein). Accordingly, the group of individuals who form the basis for the calculation of the reference level may furthermore be a group of individuals of mixed sex or same sex. Reference levels may also be obtained from the same individual as the sample YKL-40 level that is to be compared with the reference level. When this is the case the one or more reference levels may for example be YKL-40 levels measured in one or more samples obtained prior to diagnosis of the disease or disorder (pre-illness), prior to the establishment of symptoms of the disease or disorder (pre-symptom), or after a diagnosis has been established.

In a preferred embodiment of the methods of the invention, the reference level of YKL-40 is an age adjusted average level obtained by measuring the YKL-40 levels in samples from healthy individuals. In a more preferred embodiment the one or more reference levels of YKL-40 are one or more age adjusted reference levels. In an alternative embodiment the one or more reference levels is one or more previously determined levels of YKL-40 from the same subject.

Plasma YKL-40 levels increase in both sexes with increasing age and there is no difference between plasma YKL-40 in women and men. These plasma YKL-40 levels have been found from samples of and by studying a large group of healthy subjects, hereby giving a well founded reference level for plasma YKL-40 levels that may be used in the method according to the present invention (see example 1 herein).

When the present invention utilizes an age-adjusted level, then the level may be age adjusted by adding 0.5 μg/l per year for women, and 0.8 μg/l per year for men. This age-adjustment is preferably performed for a previously measured YKL-40 level from the same subject. Alternatively, the reference level may be a set of YKL-40 age dependent reference levels, e.g. one or more reference levels of YKL-40, obtained by measuring the YKL-40 levels in samples from age distributed subpopulations of healthy individuals, i.e. age specific groups of individuals as described herein above, such as e.g. individuals that are all born within the same decade. For example a set of reference levels, each being the average YKL-40 plasma level for a group of healthy individuals within the following age groups: from 30 to 39 years, from 40 to 49 years, from 50 to 59 years, and from 60 to 69 years. Preferred sets of YKL-40 age dependent reference levels are given herein further below.

Alternatively, one of the one or more reference levels of YKL-40 may be an average or median level obtained by measuring the YKL-40 levels in samples from healthy individuals, preferably the median level.

Another way of specifying a reference level is by the use of a cut-off value. A cut-off value is a value the typically divides a number of individuals into two groups: those that have an YKL-40 level above a specific cut-off value, and those that have an YKL-40 level below the specified cut-off value. The cut-off value may be used as a yes or no indicator of whether an individual is within a certain category, in relation to the present invention this corresponds to different progress and/or states of the disease, and the prognosis of the individual in question.

In a specific embodiment of the methods according to the invention, one of the one or more reference levels of YKL-40 is an age adjusted cut-off value corresponding to the 70^{th} percentile of serum or plasma YKL-40 levels in healthy individuals.

In a specific embodiment of the methods according to the invention, one of the one or more reference levels of YKL-40 is an age adjusted cut-off value corresponding to the 75^{th} percentile of YKL-40 as determined in healthy individuals.

In another specific embodiment of the methods according to the invention, one of the one or more reference levels of YKL-40 is an age adjusted cut-off value corresponding to the 85^{th} percentile of YKL-40 as determined in healthy individuals.

In another specific embodiment of the methods according to the invention, one of the one or more reference levels of YKL-40 is an age adjusted cut-off value corresponding to the 90^{th} percentile of YKL-40 as determined in healthy individuals.

In another specific embodiment of the methods according to the invention, one of the one or more reference levels of YKL-40 is an age adjusted cut-off value corresponding to the 95^{th} percentile of YKL-40 as determined in healthy individuals.

In another specific embodiment of the methods according to the invention, one of the one or more reference levels of YKL-40 is an age adjusted cut-off value corresponding to the 97.5^{th} percentile of YKL-40 as determined in healthy individuals.

Accordingly, in a preferred embodiment of the invention, the reference level of YKL-40 is an age adjusted cut-off value corresponding to the 90^{th} percentile of plasma YKL-40 in healthy individuals, such as for example a YKL-40 plasma value of 92 μg/l for a subject of about 50 years of age, or a YKL-40 plasma value of 111 μg/l for a subject of about 60 years of age; and more preferably it is an age adjusted cut-off value corresponding to the 95^{th} percentile of plasma YKL-40 in healthy individuals, such as for example a YKL-40 plasma value of 100 μg/l for a subject of about 50 years of age, or a YKL-40 plasma value of 124 μg/l for a subject of about 60 years of age. When the 95^{th} percentile plasma level is age adjusted and applied as a cut-off value, there is allowed for greater potential individual variations in the YKL-40 level. The use of the 95^{th} percentile, or even the 97.5^{th} percentile, may for instance be relevant when the methods of the invention are used in relation to severe diseases such as cancer diseases. However, in other instances of the method of the present invention it is preferred that the 90^{th} percentile plasma YKL-40 level is applied. This is e.g. when the methods are applied in relation to less severe diseases that have not yet given cause to symptoms. In the same manner, it may furthermore be relevant to utilize the 70^{th} percentile, the 75^{th} percentile, or the 85^{th} percentile of the plasma YKL-40 level in healthy individuals, which percentile is used will depend on which level of sensitivity is desired. The lower the percentile selected, as e.g. a cut-off value, the higher sensitivity is obtained. By using a low percentile subjects may be found that yet only are slightly affected by a disease or disorder, such as e.g. in an early stage of a disease or disorder. However, the lower the percentile selected the higher is the fraction of subjects that may be classified as having a disease without actually having a disease or disorder, which may be due to the potential individual biological variations.

The cut-off value may preferably be defined as a plasma YKL-40 level corresponding to the following percentiles defined in 3610 healthy subjects:

the 70% percentile (defined as: In(plasma YKL-40)=3.1+0.02×age (years)),

the 75% percentile (defined as: In(plasma YKL-40)=3.2+0.02×age (years)),

the 90% percentile (defined as: In(plasma YKL-40)=3.5+0.02×age (years)); and

the 95% percentile (defined as: In(plasma YKL-40)=3.6+0.02×age (years)) according to age.

The cut-off value may furthermore be defined as a plasma YKL-40 level corresponding to the following percentiles defined in 3610 healthy subjects:

the 70% percentile (defined as: In(plasma YKL-40)=3.1+0.02×age (years)),

the 75% percentile (defined as: In(plasma YKL-40)=3.2+0.02×age (years)),

the 85% percentile (defined as: In(plasma YKL-40)=3.4+0.02×age (years)),

the 90% percentile (defined as: In(plasma YKL-40)=3.5+0.02×age (years)),

the 95% percentile (defined as: In(plasma YKL-40)=3.6+0.02×age (years)), and

the 97.5% percentile (defined as: In(plasma YKL-40)=3.9+0.02×age (years)), according to age.

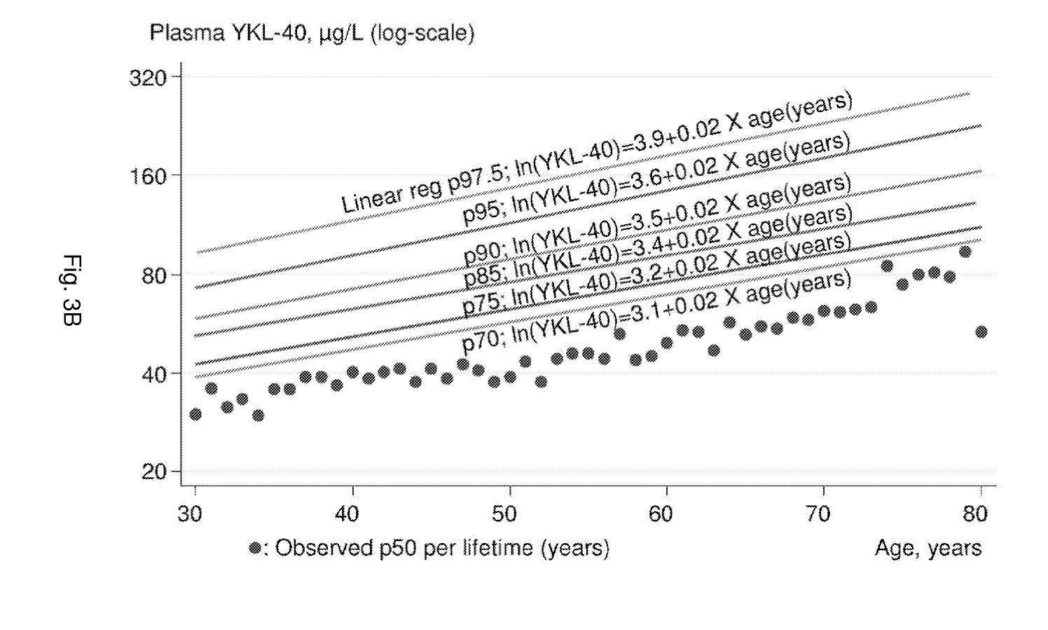

In a preferred embodiment of the methods according to the present invention the reference level of YKL-40 is calculated according to the immediately above mentioned formulas, by the use of the age of the subject. The formulas are furthermore depicted in FIG. 3A and FIG. 3B, which figures may be used in a more direct approach allowing for the determination of a cut-off value without the need for calculations. FIGS. 3A and 3B furthermore allows for an immediate comparison of a measured YKL-40 level and the subject age with e.g. both the 90^{th} percentile and the 95^{th} percentile. Hereby furthermore giving an immediate indication of the extend to which a measured YKL-40 level differs from the reference levels. By use of the above-mentioned formula for the 90^{th} percentile, the cut of value for subjects having an age of about 20 years, about 30 years, about 40 years, about 50 years, about 60 years, and about 70 years are: about 49 μg/l, about 60 μg/l, about 74 μg/l, about 90 μg/l, about 110 μg/l, and about 134 μg/lYKL-40, respectively. Correspondingly, the above mentioned formula for the 95^{th} percentile give the following cut-off values: about 55 μg/l, about 67 μg/l, about 81 μg/l, about 99 μg/l, about 122 μg/l, and about 148 μg/lYKL-40, respectively.

In one embodiment of the method according to the invention the reference level of YKL-40 is an age adjusted cut-off value corresponding to the 70^{th} percentile of serum or plasma YKL-40 levels in healthy individuals. More preferably the age adjusted cut-off value is the 70^{th} percentile defined as: In(plasma YKL-40)=3.1+0.02×age (years).

In another embodiment of the methods according to the invention the reference level of YKL-40 is an age adjusted cut-off value corresponding to the 75^{th} percentile of serum or plasma YKL-40 levels in healthy individuals. More preferably the age adjusted cut-off value is the 75^{th} percentile defined as: In(plasma YKL-40)=3.2+0.02×age (years).

In another embodiment of the methods according to the invention the reference level of YKL-40 is an age adjusted cut-off value corresponding to the 85^{th} percentile of serum or plasma YKL-40 levels in healthy individuals. More preferably the age adjusted cut-off value is the 85^{th} percentile defined as: In(plasma YKL-40)=3.4+0.02×age (years).

In another embodiment of the methods according to the invention the reference level of YKL-40 is an age adjusted cut-off value corresponding to the 90^{th} percentile of serum or plasma YKL-40 levels in healthy individuals. More preferably the age adjusted cut-off value is the 90^{th} percentile defined as: In(plasma YKL-40)=3.5+0.02×age (years).

In another embodiment of the methods according to the invention the reference level of YKL-40 is an age adjusted cut-off value corresponding to the 95^{th} percentile of serum or plasma YKL-40 levels in healthy individuals. More preferably the age adjusted cut-off value is the 95^{th} percentile defined as: In(plasma YKL-40)=3.6+0.02×age (years).

In another embodiment of the methods according to the invention the reference level of YKL-40 is an age adjusted cut-off value corresponding to the 97.5^{th} percentile of serum or plasma YKL-40 levels in healthy individuals. More preferably the age adjusted cut-off value is the 97.5^{th} percentile defined as: In(plasma YKL-40)=3.9+0.02×age (years).

In an alternative embodiment of the invention the following YKL-40 plasma levels may each independently be one of the one or more reference levels of YKL-40 to be used in a method according to the invention: a plasma level of from about 35 to about 55 μg/l, such as e.g. from about 40 to about 50 μg/l, preferably about 42 μg/l; a plasma level of from about 90 to about 100 μg/l, such as preferably about 97 μg/l; a plasma level of from about 120 to about 130 μg/l, such as preferably about 124 μg/l; and a plasma level of from about 160 to about 170 μg/l, such as preferably about 168 μg/l. These values may be used alone or in combinations of two or more of these values, such as for example as a set of reference values comprising three or more of these values. The specific values, as can be seen from the examples, have been determined from a large group of healthy individuals and correspond to the median value, the 90^{th} percentile, the 95^{th} percentile, and the 97.5^{th} percentile, respectively.

In another alternative embodiment of the methods according to the invention the one or more reference levels of YKL-40 comprises a set of reference levels of YKL-40 obtained by measuring the YKL-40 levels in samples from healthy individuals: a first reference level being the median value of YKL-40, a second reference level being the 75^{th} percentile of YKL-40, a third reference level being the 85^{th} percentile of YKL-40, a fourth reference level being the 90^{th} percentile of YKL-40, a fifth reference level being the 95^{th} percentile of YKL-40, a sixth reference level being the 97.5^{th} percentile of YKL-40 in healthy individuals, a seventh reference level being a factor 4.5 of the median value of YKL-40, and a eighth reference level being a factor 5 of the median value of YKL-40 in healthy individuals. More specifically, the median value of YKL-40 may be a plasma level of 42 μg/l, the 90^{th} percentile of YKL-40 may be a plasma level of 92 μg/l, the 95^{th} percentile of YKL-40 may be a plasma level of 124 μg/l, and the 97.5^{th} percentile of YKL-40 may be a plasma level of 168 μg/l. Furthermore, the one or more reference levels may independently be a combination of any one or more of these levels.

In a specific embodiment of the methods of the invention the reference level of YKL-40 is a set of YKL-40 age dependent cut-off values defined as two or more of the herein immediately above mentioned age adjusted cut-off value corresponding to the 70^{th}, 75^{th}, 85^{th}, 90^{th}, 95^{th}, or 97.5^{th} percentile, respectively.

In a preferred embodiment of the methods of the invention the one or more reference levels of YKL-40 is one or more of the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years), and

the 97.5^{th} percentile: In(plasma YKL-40 μg/l)=3.9+0.02×age (years).

In a more preferred embodiment of the methods of the invention the one or more reference levels of YKL-40 is one or more of the following age dependent cut-off values defined as:

the 90^{th} percentile: In(plasma YKL-40 μg/l)=3.5+0.02×age (years), and

the 95^{th} percentile: In(plasma YKL-40 μg/l)=3.6+0.02×age (years).

In another preferred embodiment of the methods of the invention, the reference level of YKL-40 is a set of YKL-40 age dependent cut-off values defined by two or more of the percentiles 70^{th}, 75^{th}, 85^{th}, 90^{th}, 95^{th}, and 97.5^{th}, as e.g. preferably calculated by the above mentioned formulas. A set of YKL-40 age dependent cut-off values may furthermore be calculated for a set of age groups, e.g. 20-29 years, 30-39 years, 40-49 years etc. where for instance the cut-off value is the highest value in the age group. In one preferred embodiment of the first or third aspect of the invention the set of cut-off values is as follows:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/ | (μg/ | (μg/ | (μg/ | (μg/l YKL- |
| (years) | l YKL-40) | l YKL-40) | l YKL-40) | l YKL-40) | 40) |
| 20-29 | 40 | 44 | 54 | 59 | 65 |
| 30-39 | 48 | 54 | 65 | 72 | 80 |
| 40-49 | 59 | 65 | 80 | 88 | 98 |
| 50-59 | 72 | 80 | 98 | 108 | 119 |
| 60-69 | 88 | 98 | 119 | 132 | 145 |
| 70-79 | 108 | 119 | 154 | 161 | 178 |
| 80-89 | 132 | 145 | 178 | 196 | 217 |

Likewise obtained by the above mentioned formulas is a more detailed set of preferred age dependent cut-off values to be used in the methods according to the present invention:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-24 | 36 | 40 | 48 | 54 | 59 |
| 25-29 | 40 | 44 | 54 | 59 | 65 |
| 30-34 | 44 | 48 | 59 | 65 | 72 |
| 35-39 | 48 | 54 | 65 | 72 | 80 |
| 40-44 | 54 | 59 | 72 | 80 | 88 |
| 45-49 | 59 | 65 | 80 | 88 | 98 |
| 50-54 | 65 | 72 | 88 | 98 | 108 |
| 54-59 | 72 | 80 | 98 | 108 | 119 |
| 60-64 | 80 | 88 | 108 | 119 | 132 |
| 65-69 | 88 | 98 | 119 | 132 | 145 |
| 70-74 | 98 | 108 | 132 | 145 | 161 |
| 75-79 | 108 | 119 | 145 | 161 | 178 |
| 80-84 | 119 | 132 | 161 | 178 | 196 |
| 85-89 | 132 | 145 | 178 | 196 | 217 |

Furthermore, the one or more reference levels of YKL-40 may be a set of YKL-40 age dependent reference levels obtained by measuring the YKL-40 levels in sample from age distributed subpopulations of healthy individuals. A preferred set of age dependent reference levels for healthy subjects can be calculated by the above formulas. Accordingly, a set of preferred age dependent reference levels to be used in the methods according to the present invention are as follows:

| | Age dependent reference levels for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 33-40 | 37-44 | 45-54 | 49-59 | 55-65 |
| 30-39 | 40-48 | 45-54 | 55-65 | 60-72 | 67-80 |
| 40-49 | 49-59 | 55-65 | 67-80 | 74-88 | 81-98 |
| 50-59 | 60-72 | 67-80 | 81-98 | 90-108 | 99-119 |
| 60-69 | 74-88 | 81-98 | 99-119 | 110-132 | 122-145 |
| 70-79 | 90-108 | 99-119 | 122-154 | 134-161 | 148-178 |
| 80-89 | 110-132 | 122-145 | 148-178 | 164-196 | 181-217 |

Likewise obtained by the above mentioned formulas is a more detailed set of preferred age dependent reference levels to be used in the methods according to the present:

| | Age dependent reference levels for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-24 | 33-36 | 37-40 | 45-48 | 49-54 | 55-59 |
| 25-29 | 37-40 | 40-44 | 49-54 | 55-59 | 60-65 |
| 30-34 | 40-44 | 45-48 | 55-59 | 60-65 | 67-72 |
| 35-39 | 45-48 | 49-54 | 60-65 | 67-72 | 74-80 |
| 40-44 | 49-54 | 55-59 | 67-72 | 74-80 | 81-88 |
| 45-49 | 55-59 | 60-65 | 74-80 | 81-88 | 90-98 |
| 50-54 | 60-65 | 67-72 | 81-88 | 90-98 | 99-108 |
| 54-59 | 67-72 | 74-80 | 90-98 | 99-108 | 110-119 |
| 60-64 | 74-80 | 81-88 | 99-108 | 110-119 | 122-132 |
| 65-69 | 81-88 | 90-98 | 110-119 | 122-132 | 134-145 |
| 70-74 | 90-98 | 99-108 | 122-132 | 134-145 | 148-161 |
| 75-79 | 99-108 | 110-119 | 134-145 | 148-161 | 164-178 |
| 80-84 | 110-119 | 122-132 | 148-161 | 164-178 | 181-196 |
| 85-89 | 122-132 | 134-145 | 164-178 | 181-196 | 200-217 |

Accordingly, by determining whether the determined level of YKL-40 in the sample is above one or more of the reference levels provides the classification of the severity of the specific disease or disorder. In other words, the classification of the specific disease or disorder is provided by comparing the determined YKL-40 level from the sample with the one or more reference levels of YKL-40, wherein the higher the level of YKL-40 the more severe the specific disease or disorder is classified as. The more severe the disease or disorder, the higher is the efficacy required of the therapy to be initiated. And likewise if the subject is already undergoing treatment the YKL-40 level is determined during monitoration of the subject, the more severe the disease and accordingly, the more severe the prognosis, the more must the ongoing treatment be altered as in administering more medicine, higher concentrations of same, or replacing the ongoing treatment for another, more efficient treatment. In other words: if the specific disease or disorder has evolved to a more severe stage of the disease or disorder and it requires a therapy of high efficacy to be initiated and/or requires a therapy with higher efficacy than the ongoing therapy to be initiated.

Another way of classifying the severity of a specific disease or disorder according to the methods of the present invention is by determining the increase in the YKL-40 level of the sample compared to a previously determined YKL-40 level from the one or more reference levels from the same subject. By determining the increase in the YKL-40 level of the sample compared to the one or more reference levels it can be determined whether a change in severity has taken place. Accordingly, in one embodiment wherein a level of YKL-40 in the sample being increased to at least a factor of 1.10 or more compared to the YKL-40 reference level indicates that a non-specific disease or disorder has evolved to a more severe stage of the disease or disorder, more preferably increased to at least a factor of 1.25, such as e.g. a factor of 1.30, or a factor of 1.40; even more preferably increased to at least a factor of 1.50, such as e.g. a factor of 1.60, a factor of 1.70, or a factor of 1.75; yet even more preferably increased to at least a factor of 1.75, such as e.g. a factor of 1.80, or a factor of 1.90, or a factor of 2; most preferably increased to at least a factor of 2, such as e.g. a factor of 2.10, a factor of 2.20, a factor of 2.25, or a factor of 2.50 compared to the YKL-40 reference level indicates that a specific disease or disorder has evolved to a more severe stage of the disease or disorder. The following is a calculation example giving a level being increased to a factor of 1.10 compared to a reference level of 50 μg/l: 50 μg/l×1.10=55 μg/l (i.e. the new level is: 55 μg/l).

In a more preferred embodiment of the first aspect of the invention a level of YKL-40 in the sample being increased by 109% compared to the YKL-40 reference level indicates that a specific disease or disorder has evolved to a more severe stage. The following is a calculation example, where the previously measured YKL-40 level is 50 μg/l, and an YKL-40 level increased by 109% is calculated: 50 μg/l+(50×1.09) μg/l=50 μg/l+54.5 μg/l=104.5 μg/l. In an increase by about 109% or more is included any method variation, biological variation or other that may influence the YKL-40 level, see example 2 herein for details.

It follows from the above that the higher the increase the stronger is the indication that a disease or disorder has evolved to a more severe stage. In a preferred embodiment of the methods of the invention a level of YKL-40 in the sample increased to a factor of 2, such as to at least a factor of 2, compared to the reference level of YKL-40 obtained as a previous measurement from the same individual, significantly indicates the worsening of a disease or disorder, i.e. that the disease or disorder has evolved to a more severe stage. An increase to at least a factor of 2 corresponds to the above-mentioned significant increase by 109% or more.

Likewise the classification of the severity of a non-specific disease or disorder according to the methods of the present invention may be performed by determining a decrease in the YKL-40 level of the sample compared to the a previously determined YKL-40 level from the same subject. Accordingly, in one embodiment wherein a level of YKL-40 in the sample being decreased at least to a factor of 0.90 compared to the YKL-40 reference level indicates that a non-specific disease or disorder has evolved to a less severe stage of the disease or disorder, more preferably decreased to least by a factor of 0.80, such as e.g. a factor of 0.70; even more preferably decreased at least to a factor of 0.60; yet even more preferably decreased at least to a factor of 0.50; most preferably decreased at least to a factor of 0.48, such as e.g. a factor of 0.45, a factor of 0.43, a factor of 0.40, or a factor of 0.38, compared to the YKL-40 reference level indicates that a non-specific disease or disorder has evolved to a less severe stage of the disease or disorder. The following is a calculation example giving a level being decreased to a factor of 0.90 compared to a reference level of 100 μg/l: 100 μg/l×0.90=90 μg/l, i.e. the new plasma YKL-40 level is 90 μg/l.

When it is written that a level is decreased at least to a factor of e.g. 0.90, it is intended to mean that the level is decreased to a factor 0.90 or e.g. 0.80, 0.70 etc., i.e., that a level of 100 μg/l is decreased to at least 90 μg/l or a lower value. Thus in line with the above increase in severity if a specific disease or disorder has evolved to a less severe stage of the disease or disorder it may thus requires a therapy of low efficacy to be initiated and/or requires a therapy with lower efficacy than the ongoing therapy to be initiated.

In a more preferred embodiment of the methods of the invention a level of YKL-40 in the sample being decreased by 52% compared to the YKL-40 reference level indicates that a specific disease or disorder has evolved to a less severe stage. The following is a calculation example, where the previously measured YKL-40 level is 100 μg/l, and an YKL-40 level decreased by 52% is calculated: 100 μg/l−(100×0.52) μg/l=100 μg/l−52 μg/l=48 μg/l. In a decrease by about 52% is included any method variation, biological variation or other that may influence the YKL-40 level, see example 2 herein for details.

It follows from the above that the greater the decrease the stronger is the indication that the disease or disorder has evolved to a less severe stage. In a preferred embodiment of the methods of the invention a level of YKL-40 in the sample decreased to a factor of 0.50, such as at least a factor of 0.50, compared to the reference level of YKL-40 obtained as a previous measurement from the same individual, significantly indicates that a change to the better has occurred, i.e. that the disease or disorder has evolved to a less severe stage. A decrease to at least a factor of 0.50 corresponds to the above-mentioned significant decrease by 52% or more.

Preferably, the previously obtained reference level of YKL-40 from the same subject, is, if necessary, an age adjusted reference level, for example obtained by adding 0.5 μg/l per year for women, and 0.8 μg/l per year for men. This may for instance be relevant when the previously obtained reference level is more than 3 years old, such as e.g. more than 5 years old, more than 8 years old, or more than 10 years old. For example when the previously obtained reference level is more than 10 years old.

In yet another embodiment of the invention, the determined level of YKL-40 in the sample is said to be above the reference level when the level of YKL-40 in the sample is increased by about 25% or more, such as e.g. by about 50% or more, about 60% or more, about 70% or more, about 80% or more, about 90% or more, about 100% or more, about 110% or more, about 120% or more, about 130% or more, or about 150% or more.

In one embodiment the one or more reference levels of YKL-40, i.e. the one or more previously determined levels of YKL-40 from the same subject, has been determined after diagnosis of the disease or disorder. In this case the method can be used to monitor the therapeutic treatment, e.g. whether the disease severity increases or decreases, and/or to determine the prognosis for the subject.

By determining the increase in the YKL-40 level of the sample compared to the one or more reference levels it can be determined whether a change in severity has taken place. Accordingly, in one embodiment of the methods of the invention wherein a level of YKL-40 in the sample being increased to at least a factor of 1.20 or more compared to the YKL-40 reference level indicates that a disease or disorder has evolved to a more severe stage of the disease or disorder, more preferably increased to at least a factor of 1.25, such as e.g. a factor of 1.30, or a factor of 1.40; even more preferably increased to at least a factor of 1.50, such as e.g. a factor of 1.60, a factor of 1.70, or a factor of 1.75; yet even more preferably increased to at least a factor of 1.75, such as e.g. a factor of 1.80, or a factor of 1.90, or a factor of 2; most preferably increased to at least a factor of 2, such as e.g. a factor of 2.10, a factor of 2.20, a factor of 2.25, or a factor of 2.50 compared to the YKL-40 reference level indicates that a disease or disorder has evolved to a more severe stage of the disease or disorder. For calculation examples, see herein above. In a more preferred embodiment a level of YKL-40 in the sample being increased by 109% or more compared to the YKL-40 reference level significantly indicates that a disease or disorder has evolved to a more severe stage of the disease or disorder and thus e.g. requires a therapy of high efficacy to be initiated and/or requires a therapy with higher efficacy than the ongoing therapy to be initiated.

Likewise a change in severity, such as e.g. lack of response of a treatment, or change to a worse prognosis, may be performed by determining a decrease in the YKL-40 level of the sample compared to the one or more reference levels. Accordingly, in one embodiment wherein a level of YKL-40 in the sample being decreased at least to a factor of 0.80 compared to the YKL-40 reference level indicates that a disease or disorder has evolved to a less severe stage of the disease or disorder, more preferably decreased at least to a factor of 0.70; even more preferably decreased at least to a factor of 0.60; yet even more preferably decreased to least by a factor of 0.50; most preferably decreased to least by a factor of 0.48, such as e.g. a factor of 0.45, a factor of 0.43, a factor of 0.40, or a factor of 0.38, compared to the YKL-40 reference level indicates that a disease or disorder has evolved to a less severe stage of the disease or disorder. For calculation examples, see herein above. In a more preferred embodiment a level of YKL-40 in the sample being decreased by 52% or more compared to the YKL-40 reference level significantly indicates that a disease or disorder has evolved to a less severe stage of the disease or disorder and thus e.g. requires a therapy of low efficacy to be initiated and/or requires a therapy with lower efficacy than the ongoing therapy to be initiated.

If a previously determined level of YKL-40 from the same subject increases by more than 0.5 μg/l per year for women, and 0.8 μg/l per year for men, then there is a risk that a disease or disorder has evolved to more severe stage. Therefore an increase, but an increase by more than the 0.5 μg/l per year for women and 0.8 μg/l per year for men, but less than the above described 109%, may be indicative for the worsening of a disease or disorder. Accordingly, if for instance a previously determined YKL-40 level was about 60 μg/l for a woman of about 25 years of age, and a new level was determined 5 years after, the increase due to age should be about 2.5 μg/l, i.e. a new age corrected value should be about 62.5 μg/l. If this value instead was measured to about 66 μg/l, it would give an indication that disease or disorder not previously present now is present or that a previous disease has become more severe. If for instance a determined YKL-40 level was about 90 μg/l for a woman of about 35 years of age with a diagnosed disease, and the YKL-40 level was determined 10 years later (45 years), the increase due to age should be about 5 μg/l, i.e. a new age corrected value should be about 95 μg/l. If this value instead was measured to e.g. 105 μg/l, it would give an indication that the disease has become more severe.

If for instance a previously determined level of YKL-40 from the same subject already was at a level where a specific disease or disorder is to be expected to be present, then an increase over time is not expected to be more than the age dependent increase of 0.5 μg/l per year for women or 0.8 μg/l per year for men; unless the specific disease or disorder is worsening. In this case it is especially preferred that the factor describing an increase is low. Accordingly, that a level of YKL-40 in the sample increased by at least a factor of 1.10 compared to the reference level of YKL-40 indicates a worsening of the non-specific disease or disorder.

Classification of Individuals

The best possible treatment is a treatment tailored to each individual, and to the stage/severity of a disease or a disorder in said individual. The present invention provides a method of classifying the severity of a specific disease or disorder, so as each individual may be classified according to e.g. a prognosis of survival. The invention further provides a method of classifying the severity of a disease or disorder, where a disease or disorder may be followed by monitoring the development of the disease or the disorder to determine whether the diseases or disorder evolve towards a more or a less severe stage of the disease or disorder. The classification and monitoration is based on the measurement of YKL-40 levels in biological samples taken from the individuals to be classified/monitored and comparing the found levels with that of one or more reference levels.

By allowing the treatment for each individual to be tailored by the classification according to severity and/or survival prognosis, both the ameliorative and the curative effect of the administered treatment will improve, the survival rate of the patients as whole improve, the relapse risks will be lowered, and the quality of life will be heightened. Furthermore, there will be a financial benefit in that the amount of drugs administered may be adjusted acutely. Also, the ability to monitor a group of individuals and determined the development in disease severity will be of assistance in choosing the most effective immediate and follow-up treatment, and be of guidance when counseling on for example required lifestyle changes.

The classification of individuals based on their YKL-40 levels may be performed according to the results described in the Examples. As can be seen from these there is a relationship between increased YKL-40 levels and increased hazard ratio of death. Hazard ratios indicate increased risk of death and are calculated as known to those skilled in the art. Accordingly, when classifying the severity of a disease or disorder according to the methods of the present invention, the severity of the disease or disorder may be deduced from cox analysis showing that patients with higher YKL-40 levels have a shorter time to disease progression and shorter time to death compared to patients/subjects with low YKL-40 levels (illustrated by the increased hazard ratio in patients with high YKL-40 levels).

The preferred groupings for the purpose of classification may be related to the age of the individuals to be classified as well disease state, future treatments and other.

A further example of a classification scheme is shown in the table below. In this example the groups are characterized by a concentration range of YKL-40 as measured in a biological sample. The ranges given in the example span increments of 25 μg/l, but may span smaller increments such as 5, 10, 15 or 20 μg/l, or alternatively span larger increments such as 30, 35, 40, 45 or 50, 60, 70 80 90 or 100 μg/l.

| | Serum YKL-40 |
|---|---|
| Group | μg/l |
| 1 | <85 |
| 2 | 85-110 |
| 3 | 110-135 |
| 4 | 135-160 |
| 5 | 160-185 |
| 6 | 185-210 |
| 7 | 210-235 |
| 8 | 235-260 |
| 9 | 260-285 |
| 10 | >285 |

Due to the relationship between YKL-40 levels in serum or plasma and the associated hazard ratios, the individuals to be classified may also be classified according to the calculated hazard ratios. A group of individuals may also be classified according to percentiles, such that the total group 100% and the 10% of the group with the lowest YKL-40 levels are group 1, the second lowest 10% percentile is group 2 and so forth. The percentiles may be 1%, 2%, 3%, 4%, 5%, 6%, 7%, 8%, 9%, 10%, 11%, 12%, 12.5%, 13%, 14%, 15%, 20%, 25%, 30%, 33% or 35% percentile groupings, or any percentile falling between or above the mentioned percentiles.

Monitoring of Individuals

The present invention relates to the monitoring of individuals based on the prognosis of their survival as measured from their YKL-40 levels. Monitoring individuals according to the measured YKL-40 levels may be used as an indication of the general state of health of an individual and/or as an indication of the effectiveness of an administered treatment. The individuals or patients may be suffering from a specific, i.e. a diagnosed disease or disorder. The specific disease or disorder may be any of the non-limiting examples: diabetes, COLD, asthma, inflammatory bowel diseases, rheumatoid arthritis, osteoarthritis, cardiovascular diseases, atherosclerosis, coronary heart disease, hypertension, liver fibrosis, acute pancreatitis, chronic pancreatitis, lung fibrosis, renal diseases, sepsis, psoriasis, etc.

Monitoring YKL-40 levels as a prognosis of death in individuals suffering from a specific disorders and/or disease facilitates administration of the most optimal treatment for each individual. The administration of an effective treatment improves both the ameliorative and curative effect of the administered treatment as well as the survival chances of the individuals, and lessens relapse risks. Thus, YKL-40 can be used for monitoring the sufficiency of medical treatment of patients with any specific disease or disorder such as, but not limited to: like diabetes, COLD, asthma, inflammatory bowel diseases, rheumatoid arthritis, osteoarthritis, cardiovascular diseases, atherosclerosis, coronary heart disease, hypertension, liver fibrosis, acute pancreatitis, chronic pancreatitis, lung fibrosis, renal diseases, sepsis, psoriasis, etc. and thus improve the curative, ameliorate and general quality of life for an individual (subject) suffering from a specific disease or disorder. Furthermore, the administration of the most effective treatment is also an issue when assessing the cost/benefits of the given treatment.

Therefore it is an aspect of the present invention to provide a method for monitoring the health state of an individual in relation to a prognosis of their survival, said method comprising: measuring the level of YKL-40 in a biological sample from said individual; and comparing the measured level to a reference level of YKL-40; wherein a statistically significant increase is an indicator for shorter survival of the individual.

Other Biomarkers

YKL-40 is an independent biomarker for classifying the severity of a disease or disorder and may be used accordingly. However, YKL-40 may also be used in combination with other known biomarkers such as C-reactive protein (CRP), ESR, carcinoembryonic antigen (CEA), CA-125, human epidermal growth factor receptor 2 (HER2), CA19-9, lactate dehydrogenase (LDH), tissue inhibitor metallo proteinase 1 (TIMP-1), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases, pentraxin 3, secretory phospholipase A2 group IIA, intercellular adhesion molecule-1, Heart-type fatty acid-binding protein (H-FABP), Myosin light chain-1 (MLC-1), P-selectin and CKMB. Of the mentioned biomarkers, both the soluble and insoluble forms of the proteins are of relevance for the present invention, such as UPAR and soluble UPAR; intercellular adhesion molecule-1 and soluble intercellular adhesion molecule-1 and others. The levels of any of the abovementioned markers may be measured in a biological sample such as a blood, serum, plasma or tissue sample and by any means available such as by use of immunoassays or PCR based assays or several assay types in combination.

It is thus furthermore an aspect of the present invention to provide means for diagnosing subjects according to their YKL-40 levels in combination with levels of other biomarkers these being selected from the non-limiting group consisting of C-reactive protein (CRP), ESR, carcinoembryonic antigen (CEA), CA-125, human epidermal growth factor receptor 2 (HER2), CA19-9, lactate dehydrogenase (LDH), tissue inhibitor metallo proteinase 1 (TIMP-1), brain natriuretic protein (BNP), interleukins and tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB; preferably C-reactive protein, ESR, carcinoembryonic antigen (CEA), CA-125, human epidermal growth factor receptor 2 (HER2), CA19-9, lactate dehydrogenase (LDH), brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB. Of these additional biomarkers C-reactive protein, brain natriuretic protein and homocysteine are of particular interest.

In a specific embodiment of this aspect of the invention the additional biomarker is selected from the group consisting of C-reactive protein, ESR, carcinoembryonic antigen (CEA), CA-125, human epidermal growth factor receptor 2 (HER2), CA19-9, lactate dehydrogenase (LDH), tissue inhibitor metallo proteinase 1 (TIMP-1), brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB; more preferably selected from C-reactive protein, brain natriuretic protein and/or homocysteine.

The above mentioned embodiments may be comprised in a kit of parts together with any required medical and or sampling equipment and instructions for use of the equipment and how to perform the assay of choice.

Biological Sample

A biological sample is a sample obtained from a subject. As such a biological sample may be a sample selected from the group consisting of tissue, blood, serum, plasma samples, urine, cerebrospinal fluid, synovial fluid, ascites, and saliva. Of special relevance to the present invention are samples of blood, serum or plasma, more preferably the biological sample is serum or plasma. Those of ordinary skill in the art will be able to readily determine which assay sample source is the most appropriate for use in the diagnosis of a particular disease, or disorder or general state of health. As there is only a minor difference between the YKL-40 levels as measured in plasma and serum, the values as described herein can be applied for both plasma and serum samples.

Subjects

The subjects herein referred to are single members of a species, herein preferably a mammalian species. Any mammalian species is an object of the present invention, although any of the following species are of particular relevance: mouse, rat, guinea pig, hamster, rabbit, cat, dog, pig, cow, horse, sheep, monkey, and human. Most preferably the subject of the present invention is a human. The subjects may in the present text also be referred to as patients or individuals.

Classification of Severity

When classifying the severity of a disease or disorder, this may for example be in relation to predetermined stages of a given disease or disorder, it may for example be in relation to a prognosis of survival, or it may be as a general evaluation of whether the disease or disorder is evolving towards a more or a less severe stage. As the prognosis of a patient may be independent of a classical staging of the disease in question, the terms “a more severe stage” and “a less severe stage”, as used herein, is also intended to mean a worsening or a bettering of the prognosis of the patient, respectively. For patients suffering from e.g. a gastrointestinal cancer disease the prognosis is typically a prognosis relating to expected time before progression, or time before death. Accordingly, a worsening of the prognosis typically corresponds to a shorter progression free interval and/or a shorter survival period.

Non-limiting examples of diseases that may be divided in stages according to severity are cancer, diabetes, COLD (chronic obstructive lung disease), asthma, inflammatory bowel diseases, rheumatoid arthritis, osteoarthritis, cardiovascular diseases, atherosclerosis, coronary heart disease, hypertension, liver fibrosis, acute pancreatitis, chronic pancreatitis, lung fibrosis, renal diseases, sepsis, psoriasis, etc.

For example, in relation to COLD, the Global Initiative for Chronic Obstructive Lung Disease has in 2007 published a report with the title: “Global Strategy for the Diagnosis, Management and Prevention of COPD”. This report gives recommendations and suggestions as to how to e.g. define, monitor and asses, and treat COLD. Especially, chapter 5, pages 31-41, relates to the classification of COLD and the assessment of severity, including the difficulties associated herewith. Different ways of measuring the progress of the disease is described in the report such as for example pulmonary function and arterial blood gas measurements; the report is incorporated herein by reference. The method according to the present invention may be used to classify the severity and at the same time used to monitor the development in the severity of COLD.

Other examples of classifying diseases, e.g. by predetermined stages, will be well-known to the skilled person within the field. This may for instance be for any disease like diabetes, COLD, asthma, inflammatory bowel diseases, rheumatoid arthritis, osteoarthritis, cardiovascular diseases, atherosclerosis, coronary heart disease, hypertension, liver fibrosis, acute pancreatitis, chronic pancreatitis, lung fibrosis, renal diseases, sepsis, psoriasis, etc.

Determination of Therapy and/or Treatment

Based upon the classification of the subject according to YKL-40 level as measured and compared to at least one reference level of YKL-40 a person skilled in the art is better equipped than ever before to determine the best possible treatment of the specific disease or disorder. Thus it is possible for the person skilled in the art based on the method herein disclosed to initiate, continue, terminate, alter or replace a therapy or therapeutic treatment in a subject suffering from the specific disease.

Following the example of above regarding COLD it will be possible for the person skilled in the art to choose the best possible treatment here fore or alternatively adapt the ongoing treatment, through monitoration of the subject during treatment.

Presently, COLD is not curable, but it is treatable. Possible treatments range from the administration of bronchodilators, beta2 agonists, M3 muscarinic antagonists, cromones, leukotriene antagonists, xanthines, corticosteroids and TNF antagonists to the administration of supplemental oxygen, and lung transplantation.

A specific example of the monitoring of chemotherapeutic treatment is given in Example 3 herein. Example 3 shows the monitoring of patients with upper gastrointestinal cancers, such as pancreatic cancers, biliary cancers and gastric cancers. The higher the YKL-40 level after a period of treatment the worse is the prognosis for survival.

Device

A fourth aspect of the present invention relates to a device for classifying the severity of a disease or disorder, wherein the device comprises means for measuring the level of YKL-40 in a sample; and means for comparing the measured level of YKL-40 with at least one reference level of YKL-40. The means for measuring the level of YKL-40 in a sample may for example be a test system that applies any of the above mentioned assay systems, such as an immunoassay, a PCR based assay or an enzymatic assay. An immunoassay is preferred for the present device.

A device according to the present invention may for example comprise a rapid, qualitative and/or quantitative test system mounted on a solid support for the determination of YKL-40 levels in biological samples.

The solid support can be used in any phase in performing any of the above assays, particularly immunoassays, including dipsticks, membranes, absorptive pads, beads, microtiter wells, test tubes, and the like. Preferred are test devices which may be conveniently used by the testing personnel or the patient for self-testing, having minimal or no previous training. Such preferred test devices include dipsticks and membrane assay systems. The preparation and use of such conventional test systems is well described in the patent, medical, and scientific literature. If a stick is used, the anti-YKL-40 antibody is bound to one end of the stick such that the end with the antibody can be dipped into or onto the biological samples. Alternatively, the samples can be applied onto the antibody-coated dipstick or membrane by pipette, dropper, tweezers or the like, or be squirted directly from the body and onto the stick. Accordingly, in a preferred embodiment of this aspect of the invention, the device is a dipstick.

In the present aspect of the invention any biological sample that is or may be converted to a fluid is preferred. Particularly biological samples that are obtainable from a body as a fluid are preferred; examples hereof include, and are not limited to: blood, serum, plasma, urine, cerebrospinal fluid, synovial fluid, ascites, semen, and saliva. More preferably serum and plasma samples.

The antibody against YKL-40 can be of any isotype, such as IgA, IgG or IgM, Fab fragments, or the like. The antibody may be a monoclonal or polyclonal and produced by methods as generally described in Harlow and Lane, Antibodies, A Laboratory Manual, Cold Spring Harbor Laboratory, 1988, incorporated herein by reference. See also section on immunoassays. The antibody can be applied to the solid support by direct or indirect means. Indirect bonding allows maximum exposure of the YKL-40 binding sites to the assay solutions since the sites are not themselves used for binding to the support. Polyclonal antibodies may be used since polyclonal antibodies can recognize different epitopes of YKL-40 thereby enhancing the sensitivity of the assay. Alternatively, monoclonal antibodies against YKL-40 may be used.

The solid support is preferably non-specifically blocked after binding the YKL-40 antibodies to the solid support. Non-specific blocking of surrounding areas can be with whole or derivatized bovine serum albumin, or albumin from other animals, whole animal serum, casein, non-fat milk, and the like.

The sample is applied onto the solid support with bound YKL-40-specific antibody such that the YKL-40 will be bound to the solid support through said antibodies. Excess and unbound components of the sample are removed and the solid support is preferably washed so the antibody-antigen complexes are retained on the solid support. The solid support may be washed with a washing solution which may contain a detergent such as Tween-20, Tween-80 or sodium dodecyl sulphate.

After the YKL-40 has been allowed to bind to the solid support, a second antibody which reacts with YKL-40 is applied. The second antibody may be labelled, preferably with a visible label. The labels may be soluble or particulate and may include dyed immunoglobulin binding substances, simple dyes or dye polymers, dyed latex beads, dye-containing liposomes, dyed cells or organisms, or metallic, organic, inorganic, or dye solids. The labels may be bound to the YKL-40 antibodies by a variety of means that are well known in the art. In some embodiments of the present invention, the labels may be enzymes that can be coupled to a signal producing system. Examples of visible labels include alkaline phosphatase, beta-galactosidase, horseradish peroxidase, and biotin. Many enzyme-chromogen or enzyme-substrate-chromogen combinations are known and used for enzyme-linked assays.

Simultaneously with the sample, corresponding steps may be carried out with a known amount or amounts of YKL-40 and such a step can be the standard for the assay. In one embodiment of the method according to the present invention the one or more reference levels of YKL-40 are reference levels for one or more predetermined stages of the disease or the disorder.

The solid support is washed again to remove unbound labelled antibody and the labeled antibody is visualized and quantitated. The accumulation of label will generally be assessed visually. This visual detection may allow for detection of different colors, e.g., red color, yellow color, brown color, or green color, depending on label used. Accumulated label may also be detected by optical detection devices such as reflectance analyzers, video image analyzers and the like. The visible intensity of accumulated label could correlate with the concentration of YKL-40 in the sample. The correlation between the visible intensity of accumulated label and the amount of YKL-40 may be made by comparison of the visible intensity to a set of reference standards. Preferably, the standards have been assayed in the same way as the unknown sample, and more preferably alongside the sample, either on the same or on a different solid support. The concentration of standards to be used can range from about 1 μg of YKL-40 per liter of solution, up to about 1 mg of YKL-40 per liter of solution, preferably the range for testing serum samples will be from 40 μg/l to 400 μg/l YKL-40. Preferably, several different concentrations of YKL-40 standards are used so that quantitating the unknown by comparison of intensity of color is more accurate. An intensity of color similar to 110 μg/l of YKL-40 may for example be considered negative, as compared with an intensity of color similar to 200 μg/l.

The device, such as the herein described dipstick or other solid support based test system, may thus be used in aid of determining the approximate level of YKL-40 in a biological sample by comparison to one or more standards/control fields. Thus the concentration of YKL-40 can be ascertained to be within a range between two of the concentrations of YKL-40 applied to the standard/control fields of the device. Alternatively the concentration of YKL-40 can be judged to be above or below a cut-off value of YKL-40, the chosen concentration for the cut-off value being applied to the control field of the dipstick. There may be multiple reference levels/standards available within and/or on the device or single reference level/standard within and/or on the device. In the latter case, the device may be used as a yes no test, to compare a YKL-level in a sample with one reference level, i.e. to see whether the YKL-level of the sample is above or below the reference level. In a preferred embodiment of a device according to the invention, the device comprises a single reference level, representing a cut-off value. The reference level may as any of the reference levels described herein above in the section termed “reference levels”.

In a preferred embodiment of the device according to the present invention the one or more reference levels of YKL-40 is one or more of the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years), and

the 97.5^{th} percentile: In(plasma YKL-40 μg/l)=3.9+0.02×age (years).

In a more preferred embodiment of the device according to the present invention the one or more reference levels of YKL-40 is one or more of the following age dependent cut-off values defined as:

the 90^{th} percentile: In(plasma YKL-40 μg/l)=3.5+0.02×age (years), and

the 95^{th} percentile: In(plasma YKL-40 μg/l)=3.6+0.02×age (years).

Although each of the steps can be carried out in the same vessel, such as a test tube, if it is cleaned and washed after each of the steps, a fast and convenient on-site assay is best performed according to the invention by using three separate vessels for each of the steps, one for the sample, one for washing, and one for developing the detectable label.

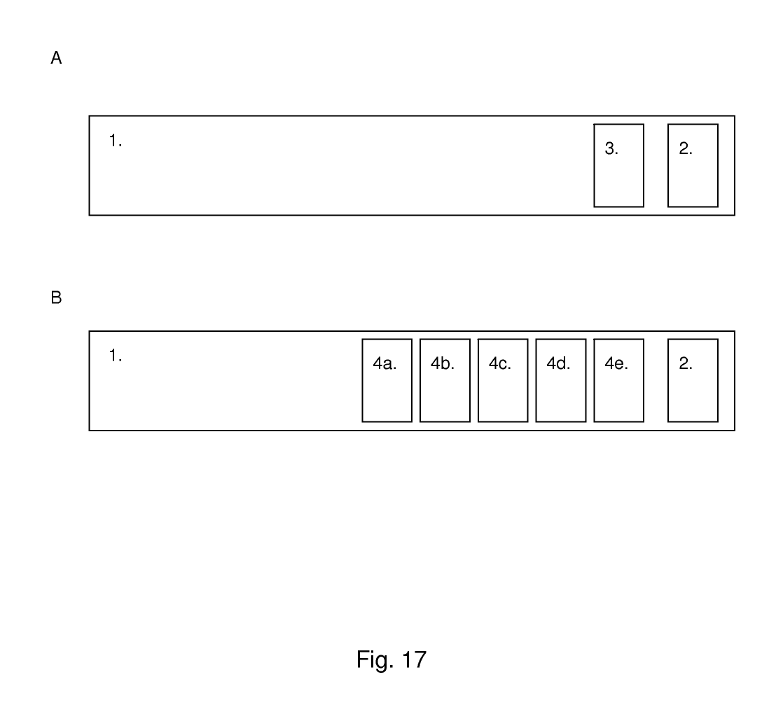

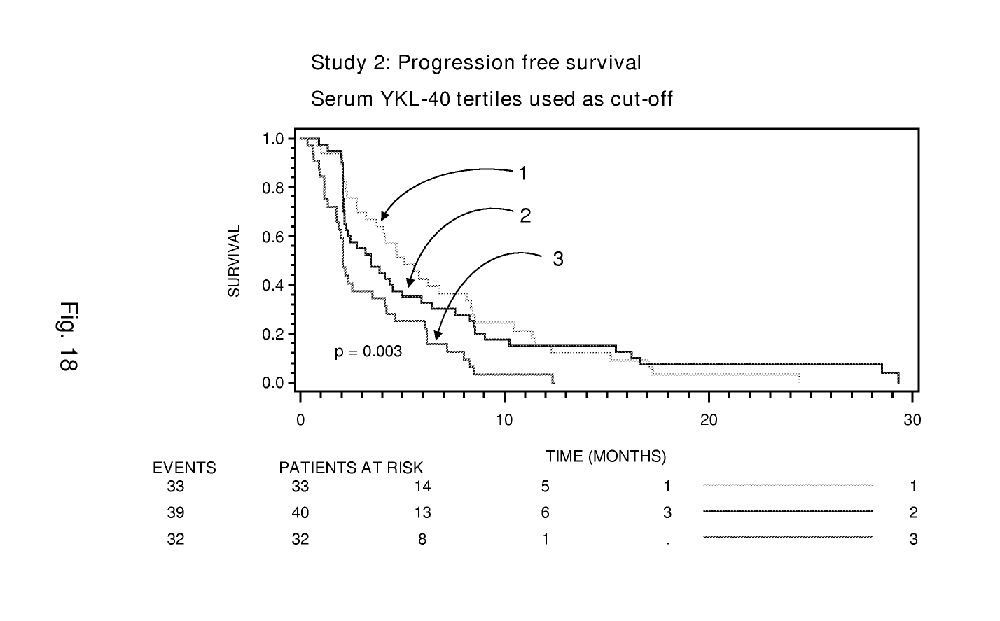

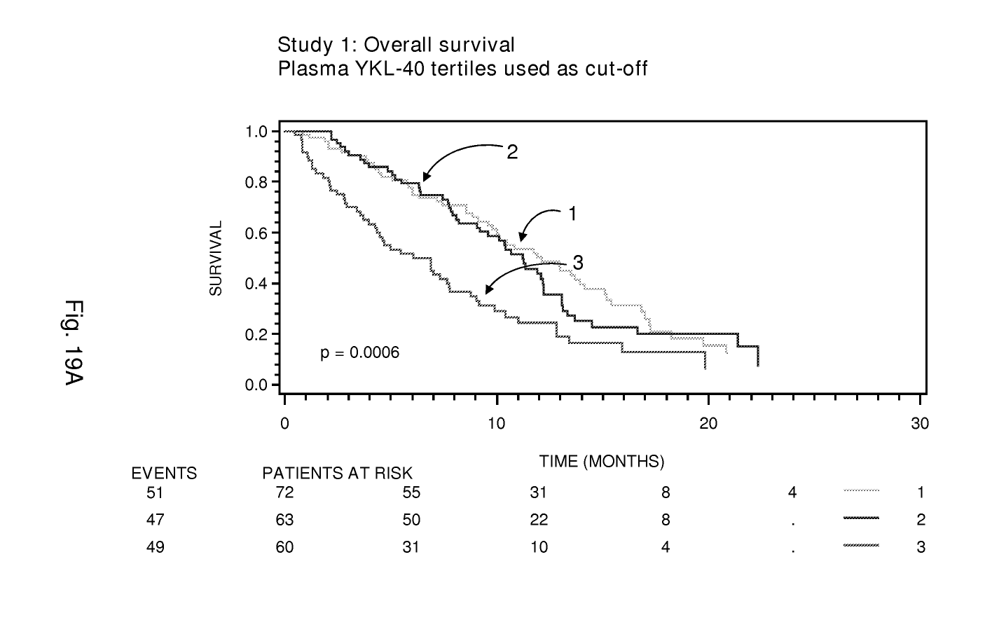

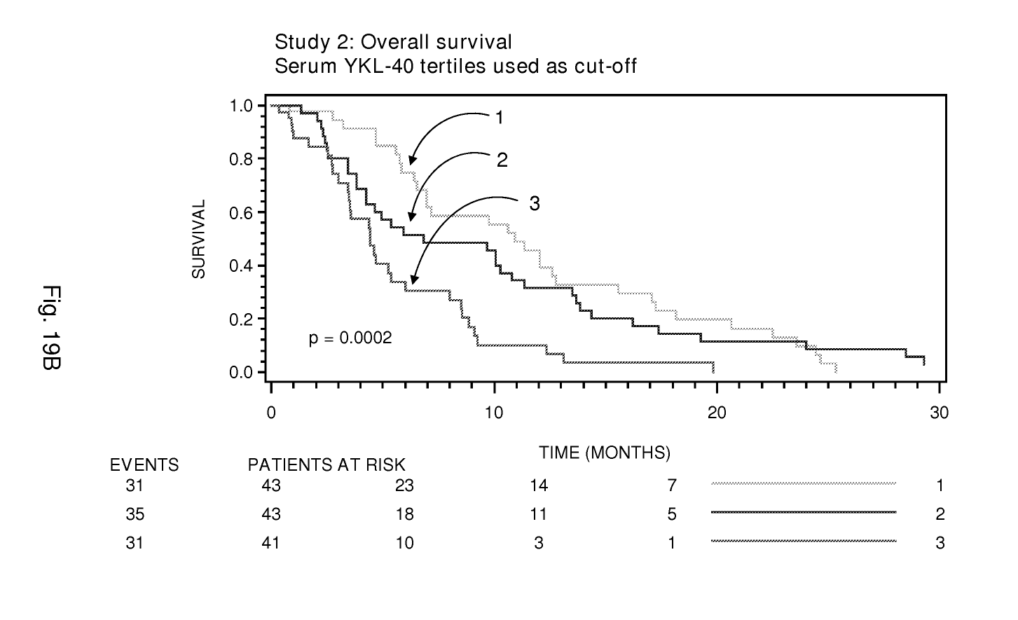

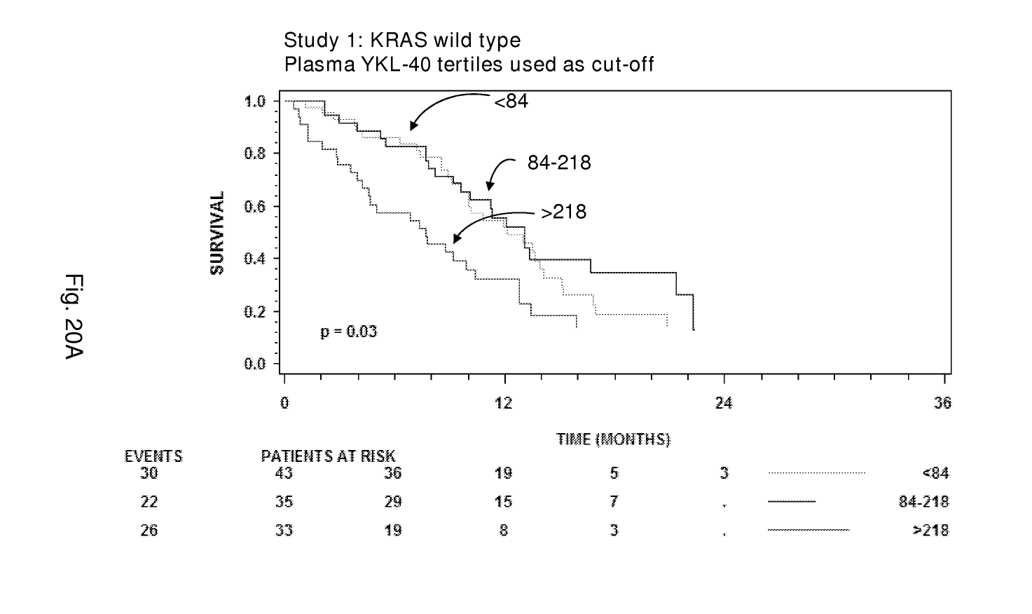

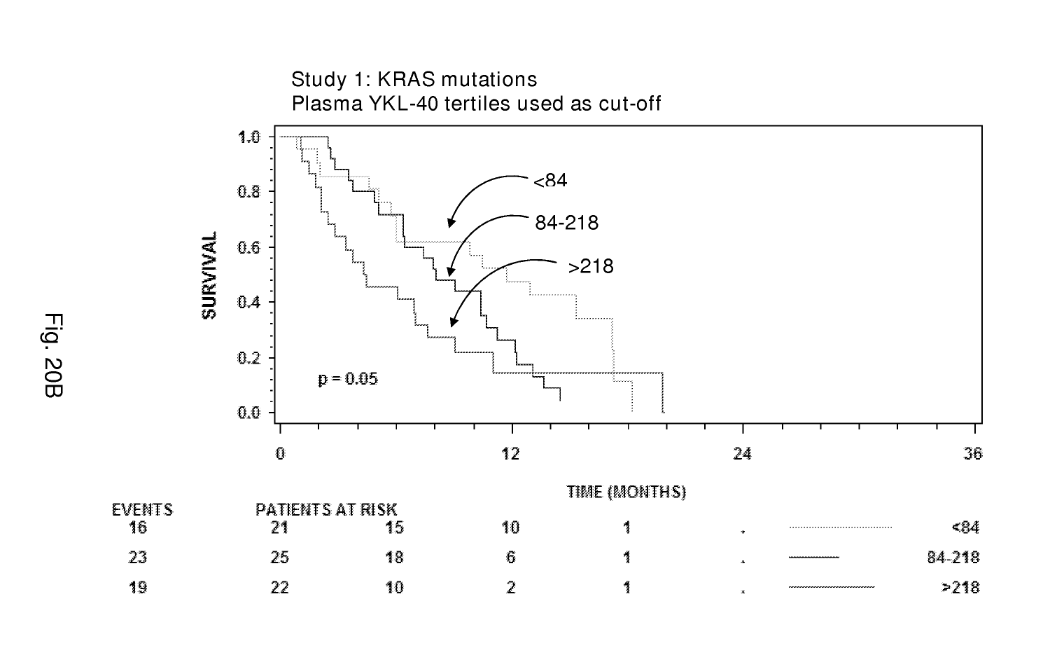

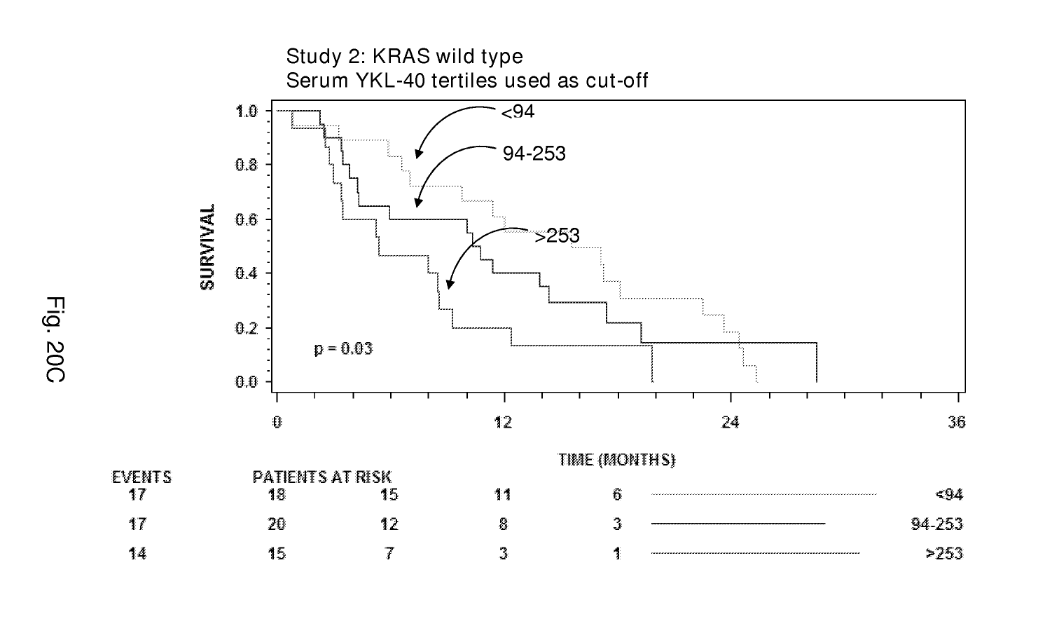

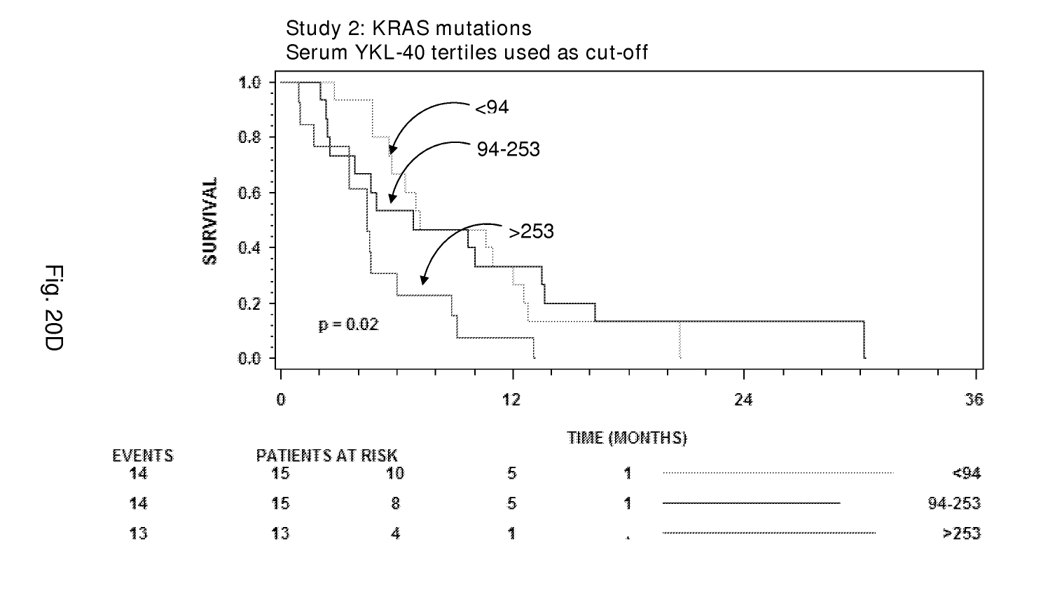

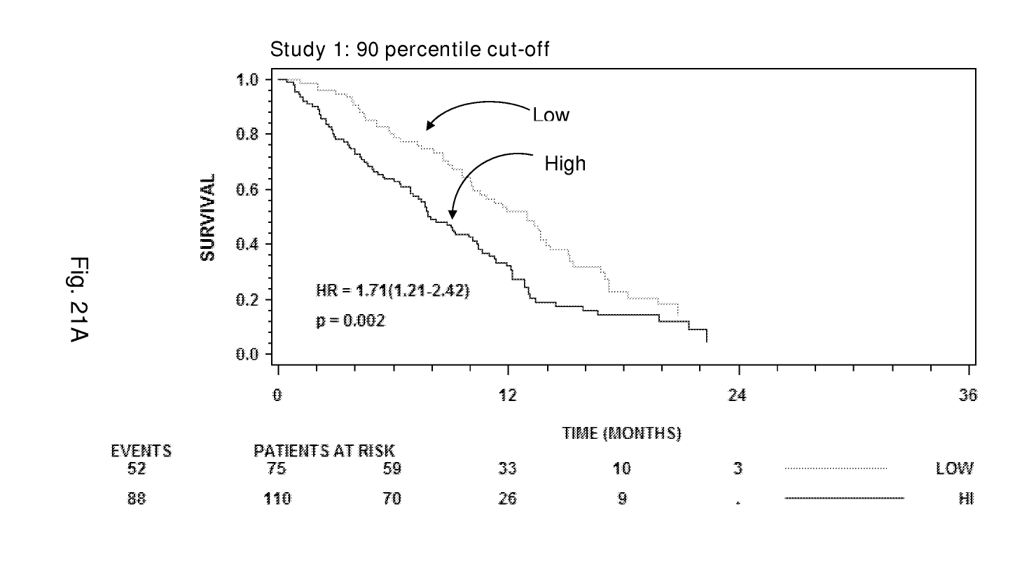

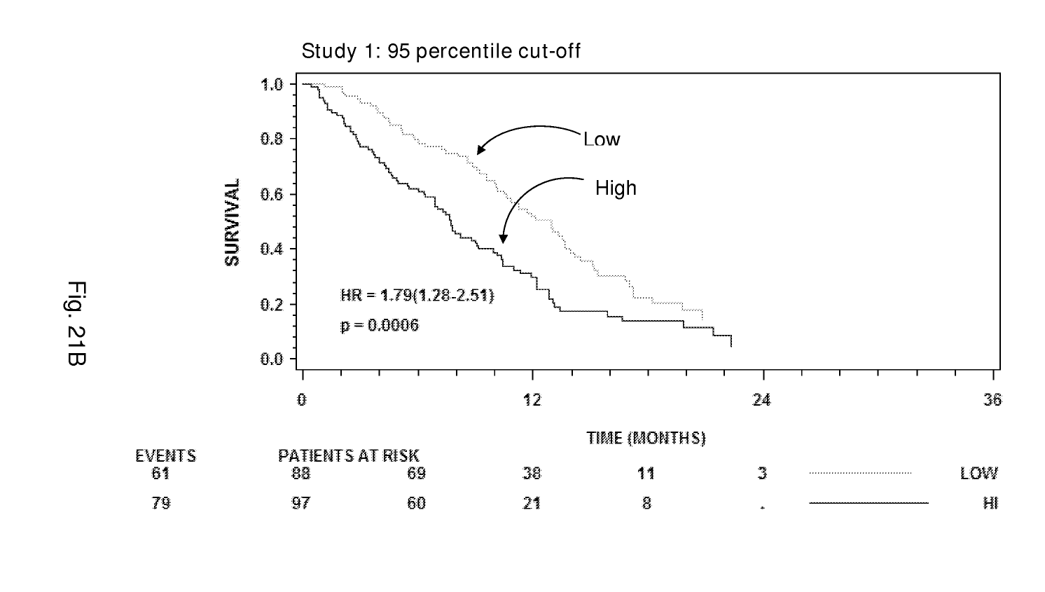

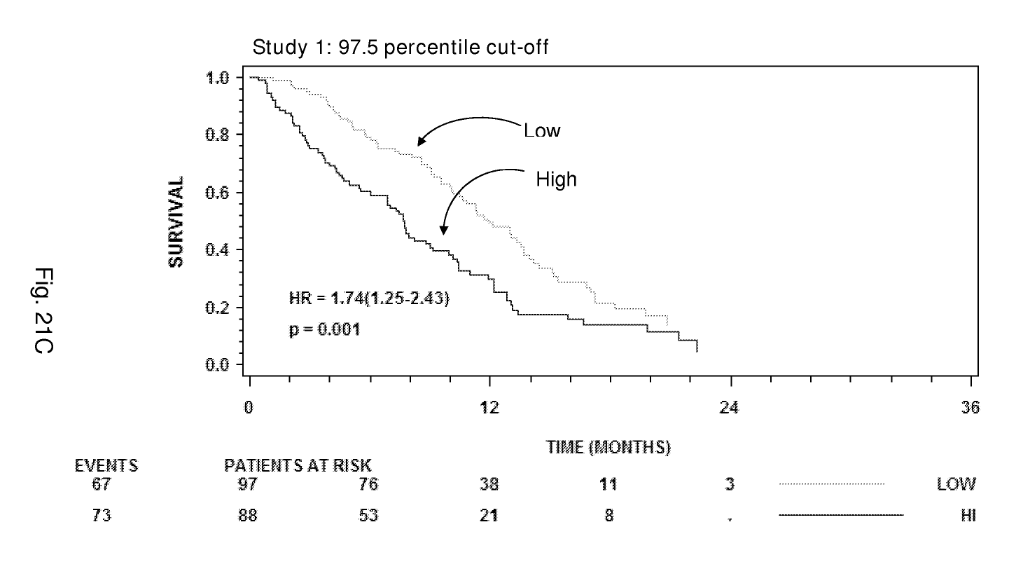

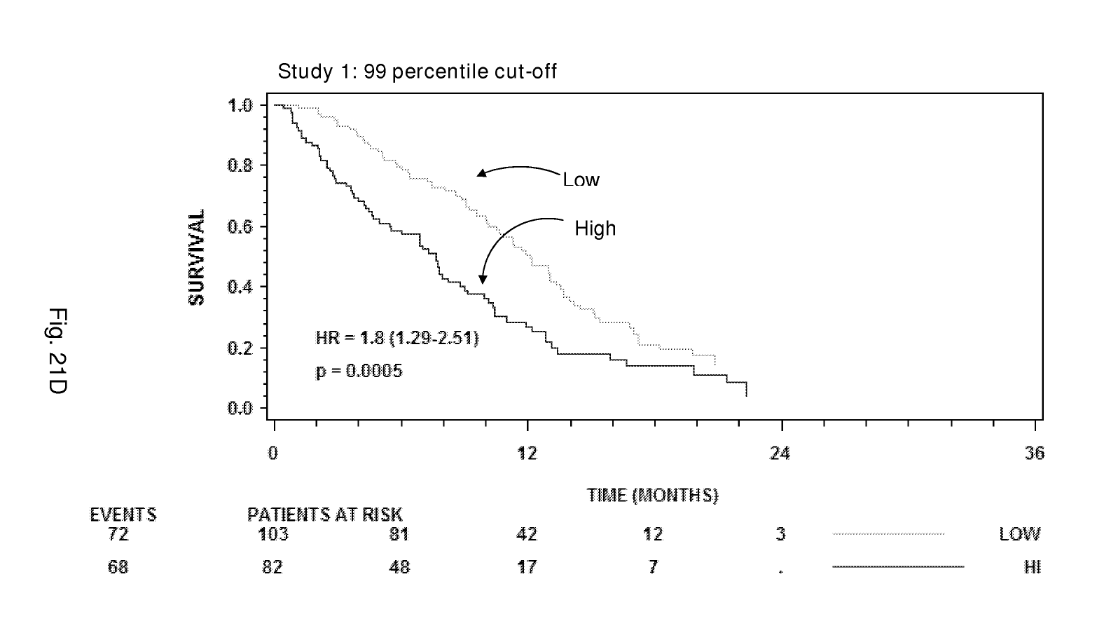

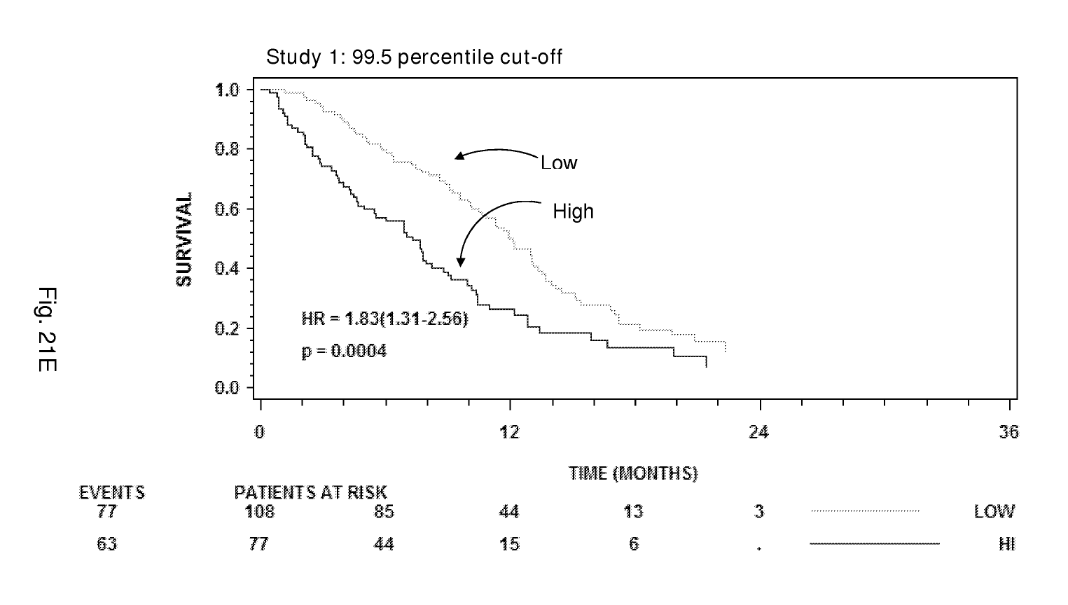

It is thus an object of the present invention that the YKL-40 level of a biological sample for use in the classification according to a reference level of YKL-40 of the individual from which the biological sample originated is measured by use of a dipstick. (see FIGS. 17A and 17B)

In an alternative embodiment of this aspect of the invention the device further comprises means for assaying additional biomarkers than YKL-40, such as any one or more of the biomarkers from the following non-limiting group: C-reactive protein (CRP), ESR, carcinoembryonic antigen (CEA), CA-125, human epidermal growth factor receptor 2 (HER2), CA19-9, lactate dehydrogenase (LDH), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases, pentraxin 3, secretory phospholipase A2 group IIA, intercellular adhesion molecule-1, Heart-type fatty acid-binding protein (H-FABP), Myosin light chain-1 (MLC-1), P-selectin and CKMB. Preferably the device comprises means for assaying C-reactive protein and/or brain natriuretic protein and/or homocysteine.

In a specific embodiment of this aspect of the invention the device comprises means for assaying additional biomarkers selected from the group consisting of C-reactive protein, ESR, carcinoembryonic antigen (CEA), CA-125, human epidermal growth factor receptor 2 (HER2), CA19-9, lactate dehydrogenase (LDH), tissue inhibitor metallo proteinase 1 (TIMP-1), brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB; more preferably means for assaying C-reactive protein, brain natriuretic protein and/or homocysteine.

The at least one reference level in relation to the device may be any reference level of YKL-40 as described herein in the section “reference levels”. In one specific embodiment of the device according to the invention, the device comprises a single reference level, representing a cut-off value.

In another specific embodiment of this aspect of the invention, the device comprises means for comparing the measured level of YKL-40 with at a set of age adjusted reference levels of YKL-40.

In another specific embodiment of this aspect of the invention, the device comprises means for comparing the measured level of YKL-40 with a set of age dependent cut-off values as defined in the following table:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 40 | 44 | 54 | 59 | 65 |
| 30-39 | 48 | 54 | 65 | 72 | 80 |
| 40-49 | 59 | 65 | 80 | 88 | 98 |
| 50-59 | 72 | 80 | 98 | 108 | 119 |
| 60-69 | 88 | 98 | 119 | 132 | 145 |
| 70-79 | 108 | 119 | 154 | 161 | 178 |
| 80-89 | 132 | 145 | 178 | 196 | 217 |

Kit of Parts

All the materials and reagents required for assaying YKL-40 according to the present invention can be assembled together in a kit, such kit includes at least elements in aid of assessing the level of YKL-40 in a biological sample obtained from an individual, and the instruction on how to do so.

Said elements may be a method of detecting the YKL-40 levels such as an immunoassay, or parts required to perform an immunoassay specific for YKL-40 detection. Optionally, a kit may further or alternatively comprise elements for performing PCR based assays for the detection of YKL-40 and determination of levels of the same from biological samples. The kit of parts may further comprise equipment for obtaining one or more biological samples, such equipment may for example be syringes, vials or other. The kit of parts may be packed for single use or for repeated usage, and the elements therein may be disposable such as to be disposed of after a single use or may be of a quality that allows repeated usage.

A fifth aspect of the present invention relates to a kit of parts comprising
i) means for measuring the level of YKL-40 in a sample;
ii) means for comparing the measured level of YKL-40 with at least one reference level of YKL-40; and
iii) instructions on how to age adjust the reference level of YKL-40, according to the age of the subject providing the sample.

The at least one reference level may be any reference level of YKL-40 as described herein in the section “reference levels”. The instructions on how to age adjust the reference level is in one embodiment of this aspect of the invention a table giving a set of age-specific subpopulations with the corresponding one or more levels of YKL-40 normal levels for healthy subjects, such as e.g. the 70^{th} percentile, the 75^{th} percentile, the 85^{th} percentile, the 90^{th} percentile and the 95^{th} percentile for healthy subjects, or any combination of one or more of these percentiles, for an example see the section “reference levels”.

In a preferred embodiment of the kit of parts according to the present invention the one or more reference levels of YKL-40 is one or more of the following age dependent cut-off values defined as:

the 70^{th} percentile: In(plasma YKL-40 μg/l)=3.1+0.02×age (years),

the 75^{th} percentile: In(plasma YKL-40 μg/l)=3.2+0.02×age (years),

the 85^{th} percentile: In(plasma YKL-40 μg/l)=3.4+0.02×age (years),

the 90^{th} percentile: In(plasma YKL-40 μg/l)=3.5+0.02×age (years),

the 95^{th} percentile: In(plasma YKL-40 μg/l)=3.6+0.02×age (years), and

the 97.5^{th} percentile: In(plasma YKL-40 μg/l)=3.9+0.02×age (years).

Means for measuring the level of YKL-40 in a sample may include one or more solutions containing a known concentration of YKL-40, a washing solution, a solution of a chromogen which changes color or shade by the action of the enzyme directly or indirectly through action on a substrate, an anti-YKL-40 antibody conjugated to a label such that it could be detected, pipettes for the transfer of said solutions, test tubes for said solutions, and a solid support, in particular adapted to be inserted into the test tubes, carrying on the surface thereof a polyclonal antibody to YKL-40. The kit may also contain one or more solid support having an anti-YKL-40 antibody for use in assaying one or more samples simultaneously or individually, and the necessary reagent required to develop the label. Included in means for comparing the measured level of YKL-40 with at least one reference level of YKL-40 may be YKL-40 standards that can be assayed fresh along with the unknown sample. Such kits will comprise distinct containers for each individual reagent.

In the above test kit, the reagents may be supplied from storage bottles or one or more of the test tubes may be prefilled with the reagents or controls.

The components of the kit may also be provided in dried or lyophilized forms. When reagents or components are provided as a dried form, reconstitution generally is by the addition of a suitable solvent. It is envisioned that the solvent also may be provided in another container means.

The kits of the present invention also will typically include a means for containing the reagents such as vials or tubes in close confinement for commercial sale such as, e.g. injection or blow-molded plastic containers into which the desired vials are retained. The kits will also comprise a set of instructions on how to perform the assay.

In an alternative embodiment of this aspect of the invention the kit will comprise means for assaying additional biomarkers than YKL-40, such as any one or more of the biomarkers from the following non-limiting group: C-reactive protein (CRP), ESR, carcinoembryonic antigen (CEA), CA-125, human epidermal growth factor receptor 2 (HER2), CA19-9, lactate dehydrogenase (LDH), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases, pentraxin 3, secretory phospholipase A2 group IIA, intercellular adhesion molecule-1, Heart-type fatty acid-binding protein (H-FABP), Myosin light chain-1 (MLC-1), P-selectin and CKMB. Preferably the kit will comprise means for assaying C-reactive protein and/or brain natriuretic protein and/or homocysteine.

In a specific embodiment of this aspect of the invention the kit comprises means for assaying additional biomarkers selected from the group consisting of C-reactive protein, ESR, carcinoembryonic antigen (CEA), CA-125, human epidermal growth factor receptor 2 (HER2), CA19-9, lactate dehydrogenase (LDH), tissue inhibitor metallo proteinase 1 (TIMP-1), brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB; more preferably means for assaying C-reactive protein, brain natriuretic protein and/or homocysteine.

The kit according to the present invention may furthermore comprise a device according to the invention as described above here in the section termed “device”.

All patent and non-patent references cited in the present application, are also hereby incorporated by reference in their entirety.

### EXAMPLES

The following examples are for illustrative purposes only and should not be construed as limiting the scope of the invention, which is defined by the appended claims.

### Example 1

Plasma YKL-40 Levels in Normal Subjects and Plasma YKL-40 as an Independent Risk Factor

Methods

Participants

We used a population-based prospective study of the Danish general population, the 1991-1994 examination of the Copenhagen City Heart Study (Bojesen et al, 2003; Nordestgaard et al, 2007; Schnohr et al, 2002). Participants aged 20 years and above were selected randomly after gender and age stratification into 5-year groups among residents of Copenhagen. Of the 17180 subjects invited, 10135 participated, and plasma was available for YKL-40 determination in 8899 participants. Participants were followed for 16 years using their unique Central Person Registry number from baseline at the 1991-1994 examination until July 2007. Follow-up was 100% complete. Roughly 99% were Caucasians of Danish descent. At time of blood sampling (1991-1994), 1763 participants had a disease known to be associated with increased levels of plasma YKL-40 (cancer, ischaemic cardiovascular disease, liver disease, diabetes, chronic obstructive pulmonary disease, asthma, rheumatoid arthritis, inflammatory bowel disease or pneumonia). During follow-up additional 3526 had developed at least one of these diseases. 3059 had died. Leaving 3610 healthy participants at the end of follow-up.

Plasma YKL-40 was measured a second time in blood samples of 929 participants of the 2001-2003 examination of the Copenhagen City Heart Study cohort. These participants were selected as having no known disease at the 1991-1994 and 2001-2003 examination, allowing correction for regression dilution bias (Clarke R, 1999).

The participants filled out a self-administered questionnaire, which was validated by the participant and an investigator on the day of attendance. Participants reported on smoking habits and subdivided into never, previous and current smoker.

Endpoints

Information on death and morbidity were collected from three different population registries using the participants' unique national Danish Central Person Registry number. Information on death was obtained from the national Danish Civil Registry System (Juel et al, 1999). Information on morbidity in ICD8 and ICD10 codes from 1976 until July 2007 was obtained from the national Danish Patient Registry (34) and subdivided into the following diagnoses associated with increased levels of plasma YKL-40: ischaemic cardiovascular disease, liver disease, diabetes, chronic obstructive pulmonary disease, asthma, rheumatoid arthritis, inflammatory bowel disease or pneumonia. Diagnoses of cancer were obtained from the national Danish Cancer Registry (from 1947 until 2004), which identifies 98% of all cancers in Denmark (35,36) and the national Danish Patient Registry (from 2004 until July 2007).

Ethics

All participants gave written informed consent. The study was approved by Herlev

Hospital and a Danish ethical committee (No. 100.2039/91 and 01-144/01, Copenhagen and Frederiksberg committee) and conducted according to the Declaration of Helsinki.

YKL-40 Analysis

Plasma levels of YKL-40 were determined in duplicates in samples frozen for 12-15 years at −80° C. by a commercial two-site, sandwich-type enzyme-linked immunosorbent assay (ELISA) (Quidel Corporation, San Diego, Calif.) (Harvey et al, 1998), using streptavidin-coated microplate wells, a biotinylated-Fab monoclonal capture antibody, and an alkaline phosphatase-labeled polyclonal detection antibody. The recovery of the ELISA was 102% and the detection limit 10 μg/L. The intra-assay coefficients of variations were 5% (at 40 μg/L), 4% (at 104 μg/L), and 4% (at 155 μg/L). The inter-assay coefficient of variation was <6%.

Statistical Analysis

We used STATA version 10.0 (Stata Corp LP, College Station, Tex.). Two-sided P<0.05 was considered significant. Mann-Whitney rank-sum test and Spearman's rho correlation were used. Plasma YKL-40 levels were stratified into categories according to plasma YKL-40 percentiles in gender and 10-year age-groups: the percentile categories were 0-33%, 34-66%, 67-90%, 91-95%, and 96-100%. In Table 3 only three percentile categories were used 0-33%, 34-90%, and 91-100%.

Kaplan-Meier curves plotted cumulative survival against left-truncated age and follow-up time in all participants. Kaplan-Meier curves also plotted cumulative survival in subgroups of participants with cancer, ischaemic cardiovascular disease, liver disease, diabetes, chronic obstructive pulmonary disease, and asthma against follow-up time. Differences between plasma YKL-40 percentile categories were examined using log-rank tests. Hazard ratios and 95% confidence intervals for death were calculated using Cox regression analysis. Hazard ratios were adjusted for other risk factors such as gender, age (deciles) and smoking habits (never/previous/current smokers) at the time of blood sampling. For trend-test, increasing plasma YKL-40 categories labelled 0, 1, 2, 3, and 4 or 0, 1, and 2 (only for the results in Table 3) were used as a continuous variable in the Cox regression. P-values for the trend-test were calculated using the Chi-square value (1 df) of the likelihood-ratio test of the model without YKL-40 categories nested in the model with YKL-40 categories. We tested for proportionality of hazards over time based on Schonefeld residuals and found no violation. Information on baseline covariates was more than 99% complete; individuals with incomplete information on covariates were excluded from multifactorial analysis. Hazard ratios were corrected for regression dilution bias using a non-parametric method (Clarke et al, 1999). For this correction we used plasma YKL-40 values from 929 healthy individuals attending both the 1991-1994 baseline examination and the 2001-2003 follow-up examination; however, the main analysis were conducted on all 8899 participants. A regression dilution ratio of 0.8042 was computed.

Absolute 10-year mortality by plasma YKL-40 percentile categories was estimated by using the regression coefficients from a Poisson regression model including the following covariates: Gender, age (<50, 50-70, >70 years), and smoking habits (never, previous, current smokers) at time of blood sampling. Absolute mortality is presented as estimated incidence rates (events/10 years) in percentages.

Results

Median survival age was 83 years for participants with plasma YKL-40 in category 0-33% and 69 years in category 96-100%. Multifactorially adjusted HRs for death were 1.2 (95% confidence interval: 1.1-1.3) for plasma YKL-40 in category 34-66%, 1.6 (1.4-1.8) for 67-90%, 2.3 (1.9-2.8) for 91-95%, and 2.8 (2.4-3.4) for 96-100% verus YLK-40 category 0-33% (p-trend=10⁻³⁷). Equivalent HRs in participants with cancer were 1.1(1.0-1.3), 1.4 (1.2-1.6), 2.1(1.5-2.8) and 2.4 (1.8-3.1) (p-trend=10¹¹), in participants with ischaemic cardiovascular disease were 1.2 (1.0-1.5), 1.5 (1.2-1.8), 2.4 (1.8-3.3) and 2.3 (1.7-3.1) (p-trend=10⁻¹³), and in participants with other diseases 1.2 (1.0-1.4), 1.4 (1.2-1.7), 2.0 (1.5-2.5) and 2.4 (1.9-3.0) (p-trend=10⁻¹⁵).

Accordingly, elevated plasma YKL-40 is associated with early death in the general population. The higher the YKL-40 level the more severe the disease or disorder stage of subject is.

Plasma YKL-40 in Healthy Participants

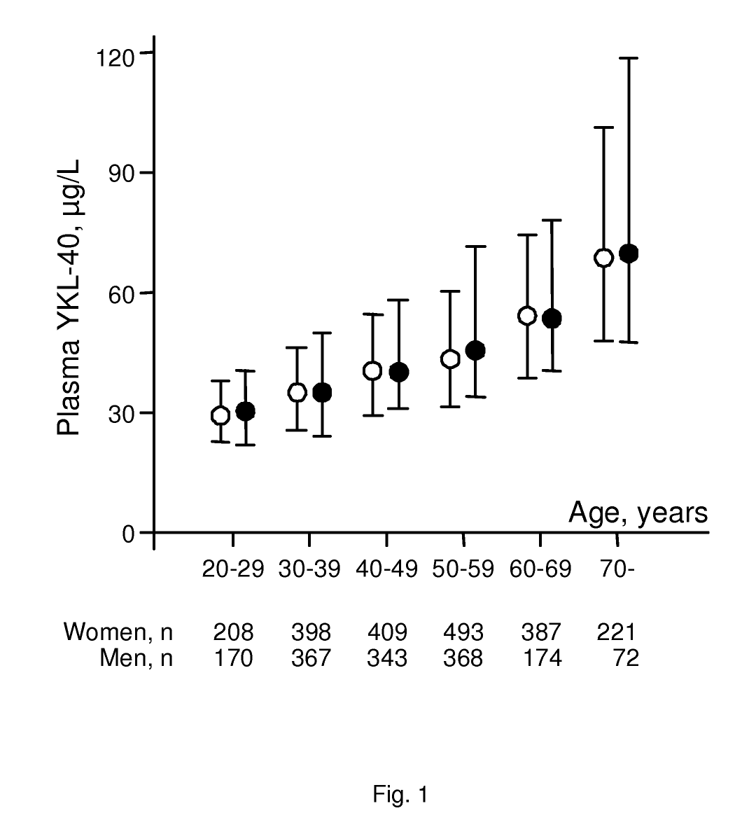

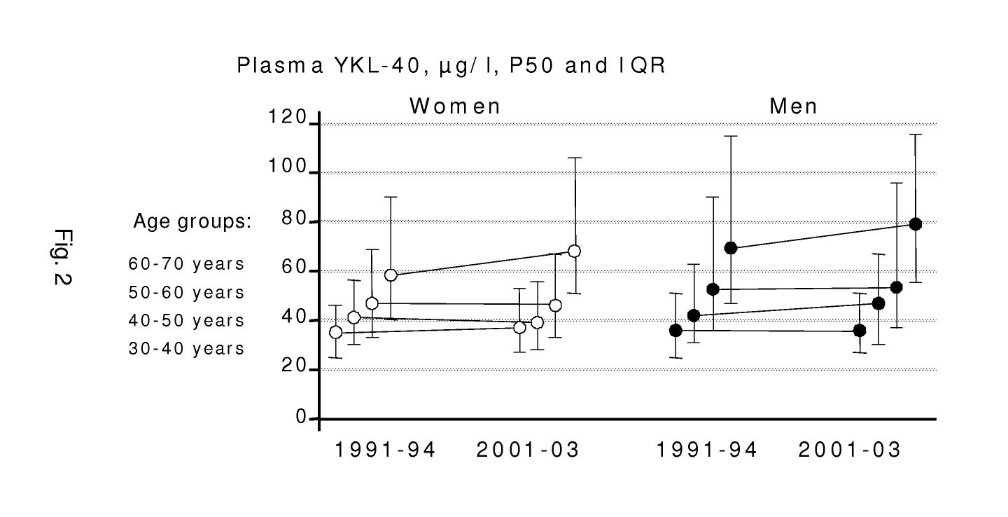

The study population consisted of 8899 participants (56% women), aged from 20 to 95 years with a mean of 59 years. Baseline characteristics of all participants according to plasma YKL-40 percentile categories adjusted for age and sex are given in Table 4. 7136 (80%) participants had no known disease at the time of blood sampling in 1991-1994. During the 16 years follow-up period 3576 developed disease leaving 3610 healthy participants at the end of follow-up. The median plasma YKL-40 in these healthy participants was 42 μg/L (2.5% -97.5% percentile range: 14-168 μg/L; 90% percentile 92 μg/L; 95% percentile 124 μg/L). Plasma YKL-40 levels increased in both sexes with increasing age (trend test p<0.0001) (FIG. 1). Spearman's rho correlation between plasma YKL-40 and age was 0.41 (p<0.0001). There was no difference between plasma YKL-40 in women and men (Mann-Whitney U; p=0.27).

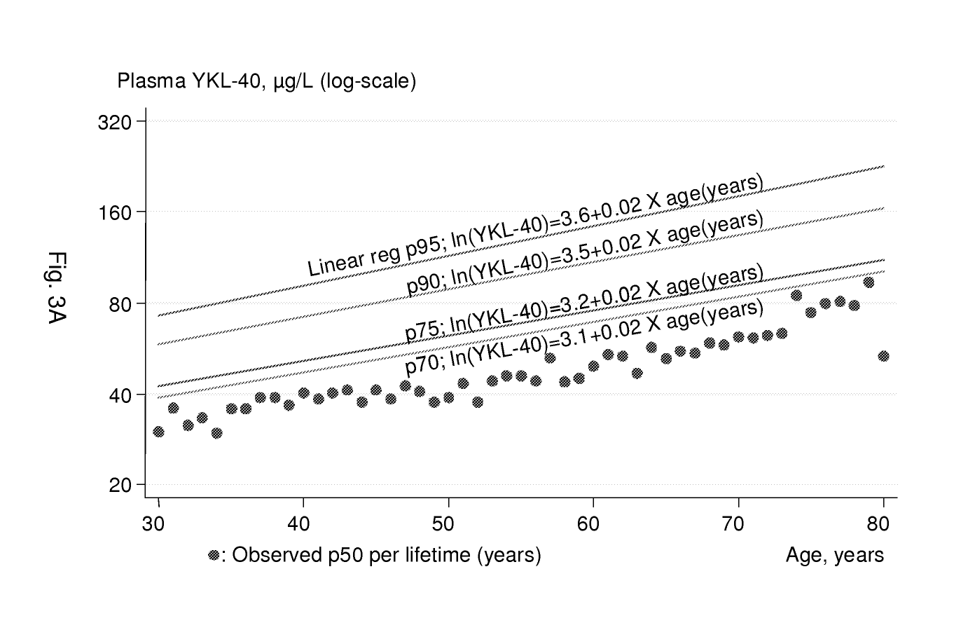

Plasma concentrations of YKL-40 in a group of 929 healthy participants (463 women and 466 men), who had their first YKL-40 measurement in the blood from the 1991-1994 examination and the second YKL-40 measurement in the blood from the 2001-2003 examination can be seen from FIG. 2. The mean increase was 0.5 μg/L/year (interquartile range −0.6-2.1 μg/L/year) in women and 0.8 μg/L/year (-0.3-2.9 μg/L/year) in men. This illustrates that plasma YKL-40 is very stable in subjects that remain healthy and a regression dilution ratio of 0.8042 was computed. There was no statistically difference between men and women.

Plasma concentrations of YKL-40 in a group of 2116 healthy women and 1494 healthy men, which had no known disease at the time of blood sampling in 1991-1994 and remained healthy during the 16 years follow-up period (i.e. none were dead or had develop cancer, ischaemic cardiovascular disease, liver disease, diabetes, chronic obstructive pulmonary disease, asthma, rheumatoid arthritis, inflammatory bowel disease, and pneumonia) can be seen from FIG. 3. The figure illustrates the mean plasma YKL-40 in these healthy participants, the 70% percentile (defined as In(plasma YKL-40)=3.1+0.02×age (years)), the 75% percentile (defined as In(plasma YKL-40)=3.2+0.02×age (years)), the 90 percentile (defined as In(plasma YKL-40)=3.5+0.02×age (years)) and the 95% percentile (defined as In(plasma YKL-40)=3.6+0.02×age (years)) according to age. Women and men were combined.

In contrast to serum CRP (Kushner et al, 2006) we found no difference in plasma YKL-40 between sexes. Furthermore, we demonstrated in a large group of healthy participants that plasma YKL-40 remained stable over time.

The median increase of plasma YKL-40 in the group of 929 healthy participants (463 women and 466 men), who had their first YKL-40 measurement in the blood from the 1991-1994 examination and the second YKL-40 measurement in the blood from the 2001-2003 examination was 0.5 pg/L/year (interquartile range −0.6-2.1 μg/L/year) in women and 0.8 μg/L/year (-0.3-2.9 μg/L/year) in men. The difference between men and women was not significant.

The median plasma concentrations of YKL-40 are higher for the participants with incident events (cancer, ischaemic cardiovascular disease, liver disease, diabetes, chronic obstructive pulmonary disease, and asthma) than for the participants who stay healthy (Table 1).

Since minor elevations in serum C-reactive protein (CRP), a inflammatory biomarker, have been shown to predict death in both healthy and diseased individuals (Kushner et al, 2006) we also examined the predictive value of plasma YKL-40 in the participants with low plasma CRP (i.e. ≦1.75 mg/L). It was examined whether the predictive value of plasma YKL-40 concentration was independent of CRP. In the 4453 participants with low plasma CRP concentrations (i.e. ≦1.75 mg/L) the hazard ratios for death were 1.0 (95% Cl, 0.8-1.2) for plasma YKL-40 percentile category 34-66%, 1.4 (1.1-1.7) for plasma YKL-40 category 67-90%, 2.3 (1.6-3.3) for category 91-95%, and 3.4 (2.5-4.8) for category 96-100% versus plasma YKL-40 percentile category 0-33% (log₁₀ p for trend 12.1). Similar results were found in the participants with plasma CRP >1.75 mg/L (log₁₀ p for trend 18.3) (Table 2). Accordingly, in these subjects the hazard ratios for death increased highly significant with increasing plasma YKL-40 levels, confirming that plasma YKL-40 is independent of plasma CRP.

Elevated plasma YKL-40 and increased risk of death was not related to a specific type of disease, but was found in participants diagnosed with cancer, ischaemic cardiovascular disease, liver disease, diabetes, and chronic obstructive pulmonary disease either before the time of blood sampling in 1991-1994 or during the 16 years follow-up period.

The association between increasing plasma YKL-40 and increased risk of death was similar, or higher, than that of smoking status and risk of death. Furthermore, multivariate cox analysis including smoking status, age and sex demonstrated that plasma YKL-40 was an independent risk factor, i.e. it was shown that plasma YKL-40 percentile category was a risk factor for early death independent of age, gender, plasma CRP, smoking status or disease (cancer, ischemic cardiovascular disease, and other diseases associated with elevated plasma YKL-40). Increasing plasma YKL-40 was associated with smoking (trend, p=0.0005).

In this study of adults from the Danish general population we found that elevated plasma concentrations of YKL-40 predicted early death. The difference in the median survival age between participants with elevated plasma YKL-40 compared to low plasma YKL-40 was 14 years, and the difference in the percentage of participants alive at 15-years follow-up after the time of blood sampling between these two groups was 26%.

It is a strength of the study that the predictive value of plasma YKL-40 was evaluated in a large cohort of well characterized subjects, with a long follow-up period, and with no losses to follow-up.

Plasma YKL-40 as a Risk Factor of Death in the General Population

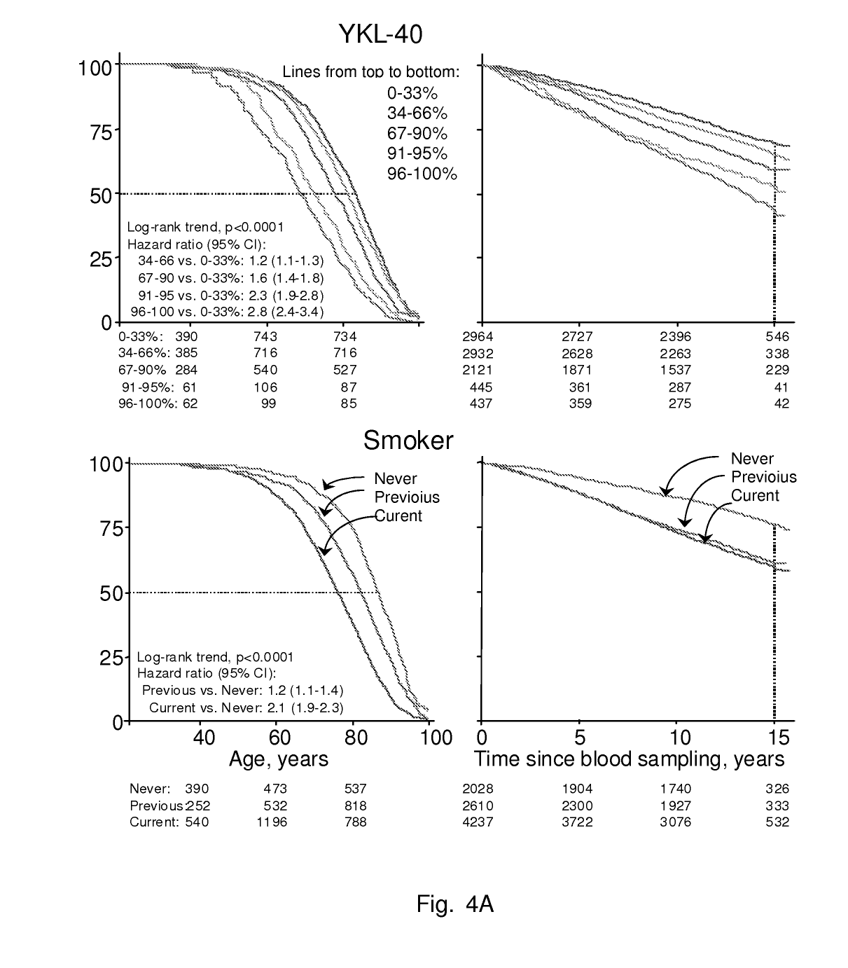

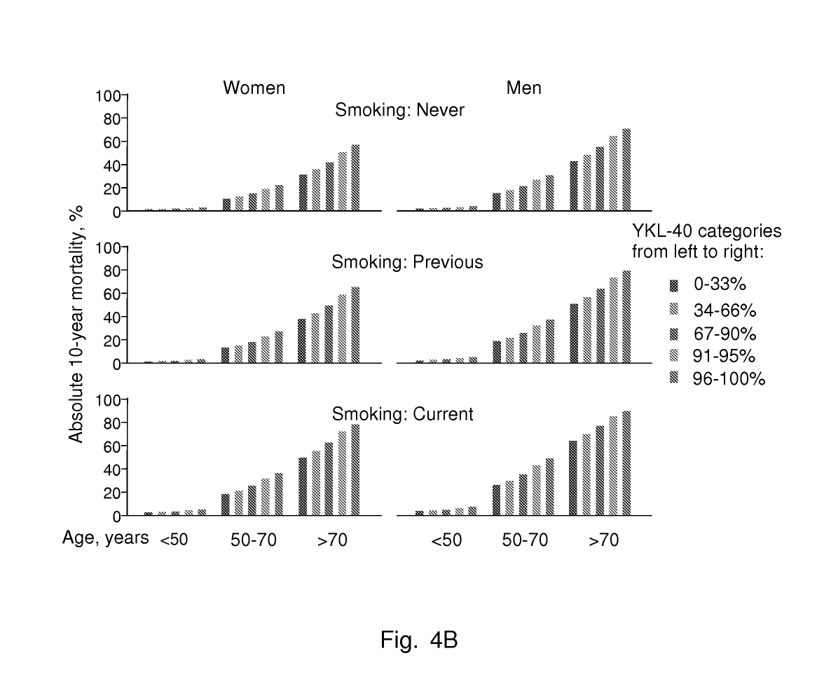

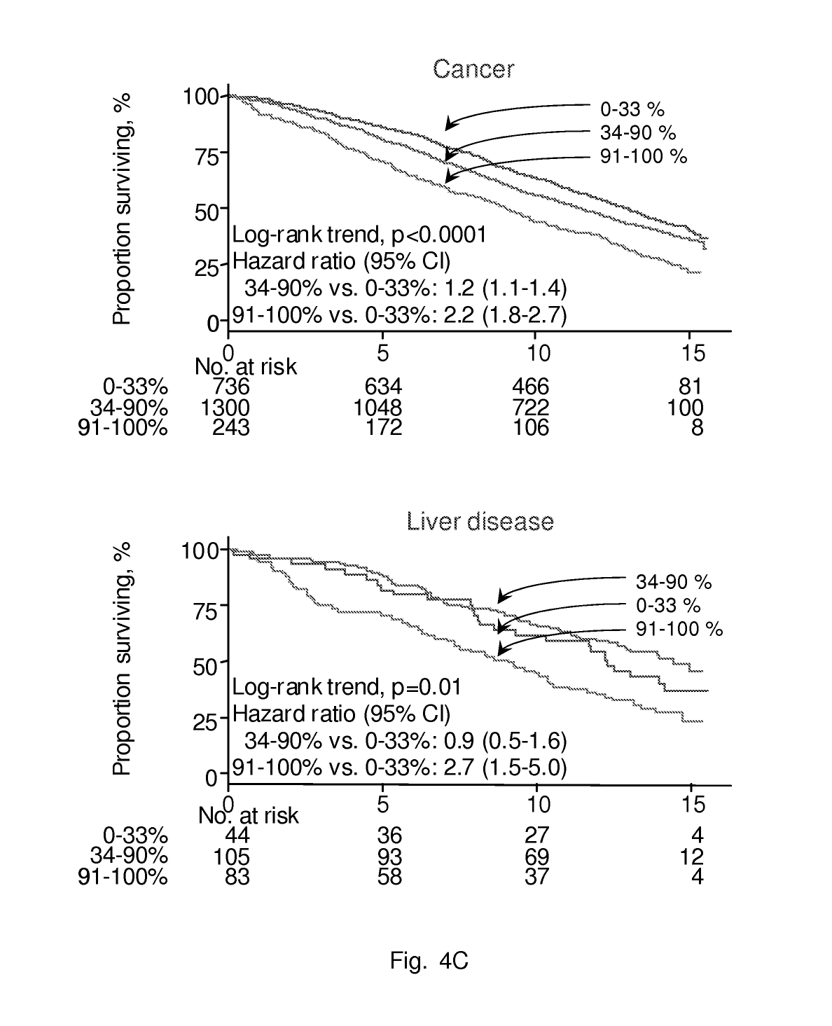

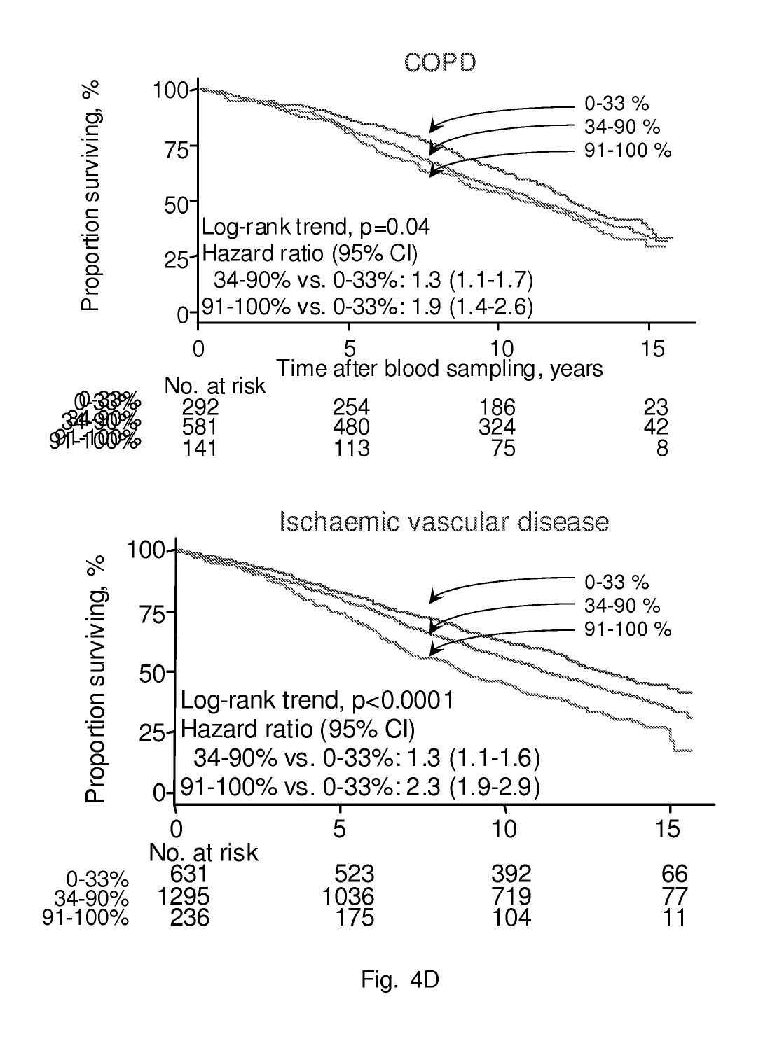

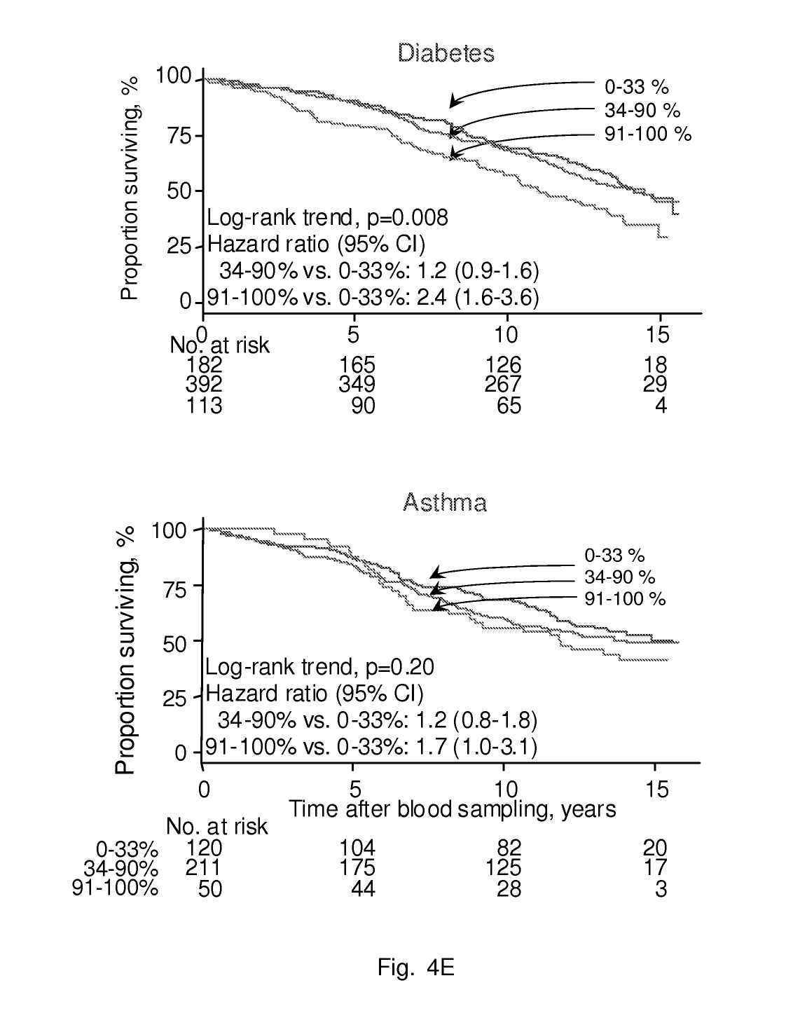

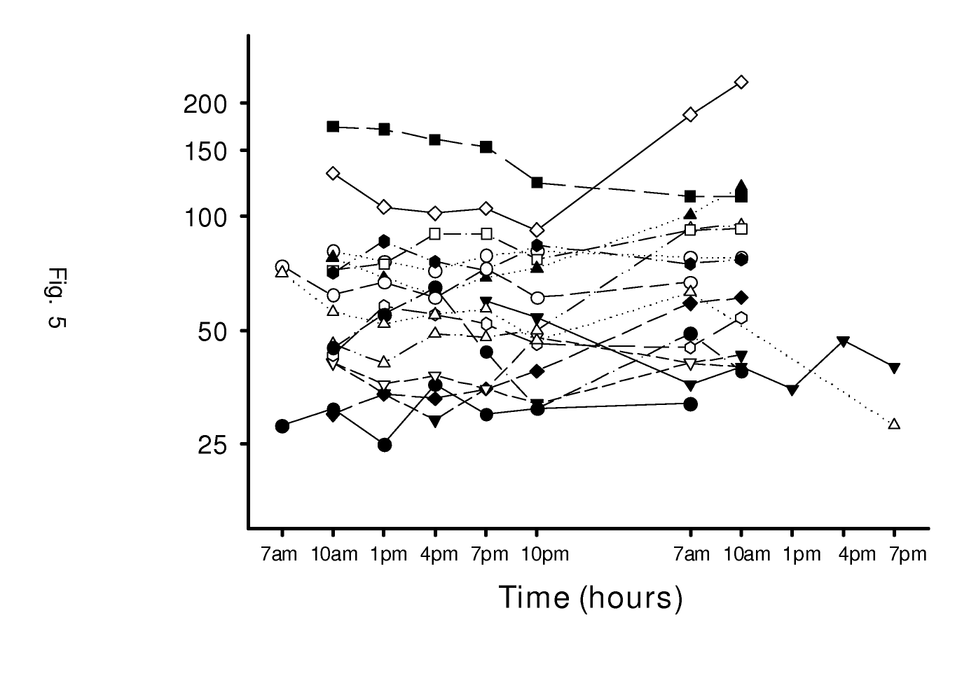

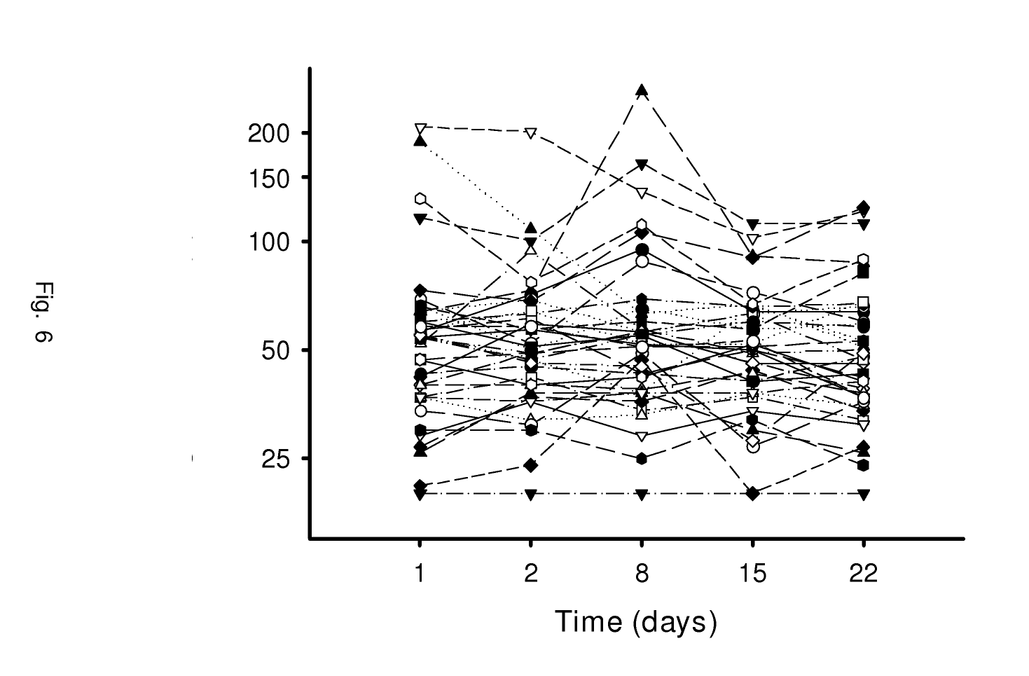

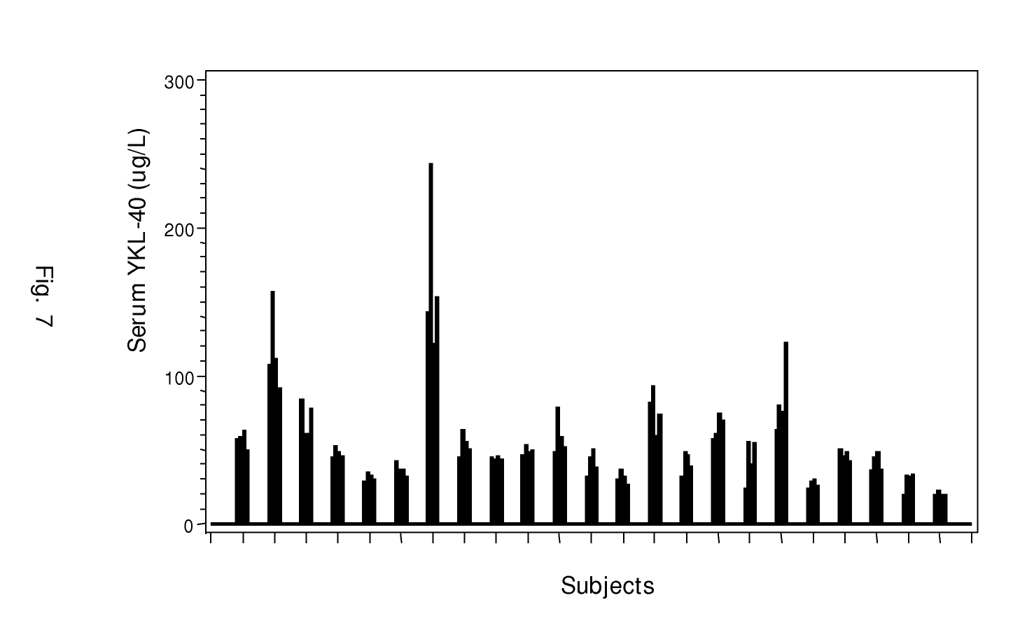

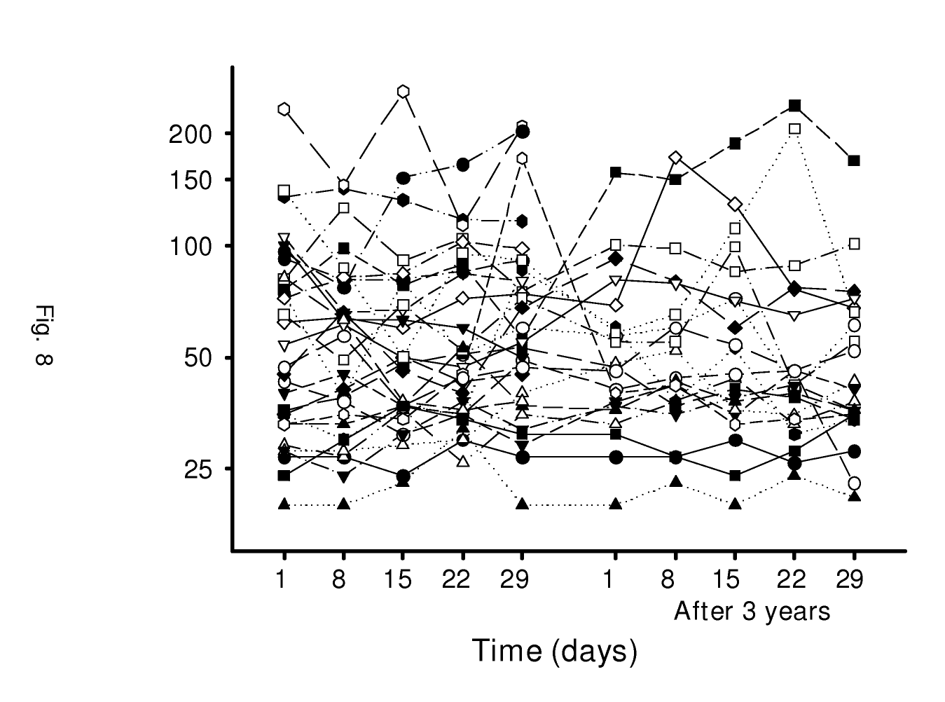

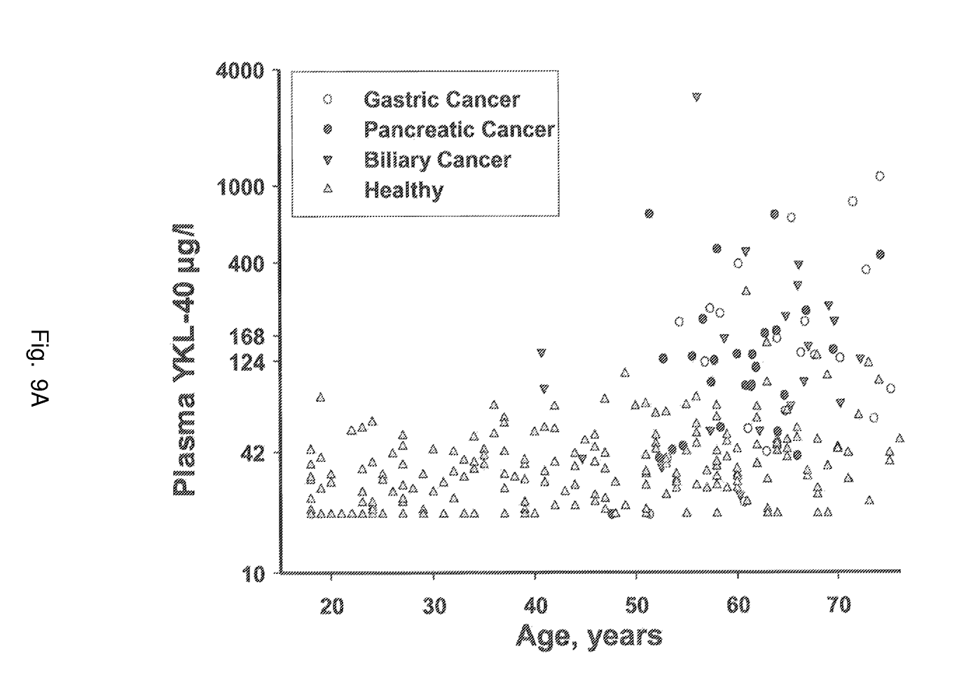

During 16 years follow-up, 3059 of the 8899 participants died. Increasing plasma YKL-40 (divided into five gender and 10-year age percentile categories) was associated with increasing risk of early death of all causes (log rank test, p=3.8*10⁻⁴⁶) (Table 3 and FIG. 4A). Participants with low plasma YKL-40 (percentile 0-33%) vs. participants with high plasma YKL-40 (percentile 96-100%) had a longer median survival age of 83 years vs. 69 years and a higher 15-year survival of 70% vs. 44%. Thus, the effect on median survival age and 15-year survival of increasing plasma YKL-40 was similar or even higher than that of smoking status (Table 3 and FIG. 4A).

Multifactorially adjusted (sex, age, and smoking status at time of blood sampling) hazard ratios for overall death were 1.2 (95% Cl, 1.1-1.3) for plasma YKL-40 percentile category 34-66%, 1.6 (1.4-1.8) for 67-90%, 2.3 (1.9-2.8) for 91-95%, and 2.8 (2.4-3.4) for plasma YKL-40 percentile category 96-100% versus plasma YKL-40 percentile category 0-33% (p-trend, p=1.0*10⁻³⁷). These estimates remained constant after adjusting for violent death (Table 2). Hazard ratios (HR) for death were calculated according to plasma YKL-40 in gender and 10-year age percentile categories.

Plasma YKL-40 as a Risk Factor of Death in Participants with Known (at Time of Follow-Up) Cancer, Ischaemic Cardiovascular Disease, Liver Disease, Diabetes, Chronic Obstructive Pulmonary Disease and Asthma

Increasing plasma YKL-40 (divided into three gender and 10-year age percentile categories) was associated with increasing risk of death in participants with cancer (p<0.0001), ischaemic cardiovascular disease (p<0.0001), liver disease (p=0.01), diabetes (p=0.008), and chronic obstructive pulmonary disease (p=0.04), whereas no association was found in participants with asthma (FIG. 4C-E). The participants had these diagnoses either at time of blood sampling between1991-1994 or during the follow-up period. Participants with cancer and plasma YKL-40 in percentile category 91-100% had the shortest survival with a hazard ratio of 2.2 (1.8-2.7) compared to participants with cancer and plasma YKL-40 in percentile category 0-33% (FIG. 4C). Similar results were found in participants with ischaemic cardiovascular disease with a hazard ratio of 2.3 (1.9-2.9) for plasma YKL-40 in percentile category 91-100% compared to plasma YKL-40 in percentile category 0-33%, liver disease 2.7 (1.5-5.0), diabetes 2.4 (1.6-3.6), and chronic obstructive pulmonary disease 1.9 (1.4-2.6) (FIG. 4C-D).

In participants with cancer, in participants with ischaemic cardiovascular death and in participants with other diseases, highly significant associations were also found between increasing plasma YKL-40 percentile categories and increasing multifactorially adjusted hazard ratios for risk of death (log₁₀ p for trend 11.4, 12.5, and 15.1, respectively) (Table 2).

In order to verify that plasma YKL-40 was not just another marker of inflammation, we examined if the predictive value of plasma YKL-40 concentration was independent of the inflammatory biomarker, C-reactive protein (CRP). Interestingly, in the 4453 participants with low plasma CRP concentrations (i.e. ≦1.75 mg/L) the hazard ratios for death were 1.0 (95% Cl, 0.8-1.2) for plasma YKL-40 percentile category 34-66%, 1.4 (1.1-1.7) for plasma YKL-40 category 67-90%, 2.3 (1.6-3.3) for category 91-95%, and 3.4 (2.5-4.8) for category 96-100% versus plasma YKL-40 percentile category 0-33% (log₁₀ p for trend 12.1). Similar results were found in the participants with plasma CRP >1.75 mg/L (log₁₀ p for trend 18.3) (Table 2).

Absolute 10-Year Mortality

The lowest absolute 10-year mortality was 1.2% in never smoking women aged <50 years in the plasma YKL-40 percentile category 0-33% (FIG. 4B). Absolute 10-year mortality was higher in men than in women and increased with increasing age and from never through previous to current smoking status. The highest absolute 10-year mortality was 78% and 90% in smoking women and men aged >70 years and in the 96-100% plasma YKL-40 percentile category (FIG. 4B).

In conclusion, in this large prospective study of subjects from the general population we found a strong association between elevated plasma concentrations of YKL-40 and early death, independent of smoking.

TABLE 1
Status of study participants from the general population and plasma YKL-40 concentration

| | | Participants with event during follow-up | | | | | | |
|---|---|---|---|---|---|---|---|---|
| | | | Sex and 10-year age-groups percentiles of plasma | | | | | During |
| | At blood | Median | YKL-40, n (%) | | | | | follow- |
| Status | sampling, n | (IQR), μg/l | 0-33% | 34-66% | 67-90% | 91-95% | 96-100% | up, n |
| Healthy | 7136 | 42 (30-61) | 1364 (38) | 1247 (35) | 759 (21) | 138 (4) | 102 (3) | 3610 |
| Any disease* | 1763 | 67 (42-110) | 1121 (32) | 1117 (32) | 883 (25) | 207 (6) | 198 (6) | 3526 |
| Cancer | 704 | 65 (42-107) | 528 (34) | 509 (32) | 376 (24) | 83 (5) | 79 (5) | 1575 |
| Ischaemic | 664 | 73 (46-116) | 455 (30) | 491 (33) | 397 (27) | 79 (6) | 76 (5) | 1498 |
| cardiovasc, disease | | | | | | | | |
| Liver disease | 81 | 96 (49-217) | 30 (20) | 37 (25) | 27 (18) | 20 (13) | 37 (25) | 151 |
| Diabetes | 156 | 71 (45-128) | 147 (28) | 159 (30) | 147 (28) | 36 (7) | 42 (8) | 531 |
| Chronic obstruct, | 155 | 71 (46-122) | 252 (29) | 251 (29) | 237 (28) | 51 (6) | 68 (8) | 859 |
| pulm, disease | | | | | | | | |
| Asthma | 93 | 56 (39-96) | 98 (34) | 88 (31) | 67 (23) | 20 (7) | 15 (5) | 288 |

*Death (only incident), cancer, ischaemic cardiovascular disease, liver disease, diabetes, chronic obstructive pulmonary disease, asthma, rheumatoid arthritis, inflammatory bowel disease, pneumonia. Some participants had more than one disease.
IQR, interquartile range.

TABLE 2
Hazard ratios for death and plasma YKL-40 concentration

| | | Hazard ratio* by sex and 10-year age-groups percentiles | | | | | |
|---|---|---|---|---|---|---|---|
| | Participants/ | of YKL-40 | | | | | −log₁₀ |
| Population/Event | Events | 0-33% | 34-66% | 67-90% | 91-95% | 96-100% | (p-trend) |
| All§/Death | 8875/3047 | 1.0 | 1.2 (1.1-1.3) | 1.6 (1.4-1.8) | 2.3 (1.9-2.8) | 2.8 (2.4-3.4) | 37.3 |
| All§/Non-violent death | 8804/2976 | 1.0 | 1.2 (1.1-1.3) | 1.6 (1.4-1.8) | 2.3 (1.9-2.8) | 2.8 (2.4-3.4) | 36.8 |
| All§/Violent death | 8875/71 | 1.0 | 1.6 (0.8-3.2) | 1.2 (0.5-2.8) | 1.9 (0.5-7.1) | 2.6 (0.8-8.8) | 0.7 |
| Never-smokers/Death | 2028/450 | 1.0 | 1.1 (0.8-1.4) | 1.6 (1.2-2.2) | 2.5 (1.5-4.2) | 3.6 (2.1-6.1) | 7.2 |
| Ever-smokers/Death | 6847/2597 | 1.0 | 1.2 (1.1-1.4) | 1.6 (1.4-1.8) | 2.2 (1.8-2.7) | 2.7 (2.2-3.3) | 30.4 |
| Plasma CRP-conc.¶ ≦1.75 mg/L/Death | 4453/1081 | 1.0 | 1.0 (0.8-1.2) | 1.4 (1.1-1.7) | 2.3 (1.6-3.3) | 3.4 (2.5-4.8) | 12.1 |
| Plasma CRP-conc.¶ >1.75 mg/L/Death | 4404/1958 | 1.0 | 1.3 (1.1-1.5) | 1.5 (1.3-1.8) | 2.1 (1.6-2.6) | 2.4 (1.9-3.0) | 18.3 |
| Participants with cancer§/Death | 2271/1400 | 1.0 | 1.1 (1.0-1.3) | 1.4 (1.2-1.6) | 2.1 (1.5-2.8) | 2.4 (1.8-3.1) | 11.4 |
| Participants with ischaemic cardiovascular | 2158/1327 | 1.0 | 1.2 (1.0-1.5) | 1.5 (1.2-1.8) | 2.4 (1.8-3.3) | 2.3 (1.7-3.1) | 12.5 |
| disease§/Death | | | | | | | |
| Participants with other diseases§**/Death | 2820/1599 | 1.0 | 1.2 (1.0-1.4) | 1.4 (1.2-1.7) | 2.0 (1.5-2.5) | 2.4 (1.9-3.0) | 15.1 |

§For 24 participants smoking status was unknown.
¶For additional 18 participants plasma concentration of CRP was unknown.
*Hazard ratios were adjusted for other risk factors such as gender, age (deciles) and smoking habits (never/previous/current smokers) at time of blood sampling, corrected for regression dilution bias.
CRP = C-reactive protein.
**Benign liver disease, diabetes, chronic obstructive pulmonary disease and asthma, rheumatoid arthritis, inflammatory bowel diasease, pneumonia. Some participants had more than one disease.

TABLE 3
Median survival age and 15-year survival in participants
from the general population according to plasma YKL-40
percentile category or smoking status#.

| Risk | Median survival age, years | 15-year survival, % |
|---|---|---|
| factor | (95% confidence interval) | (95% CI) |
| YKL-40 | | |
| 96-100% | 69 (66-72) | 44 (39-49) |
| 91-95% | 73 (69-75) | 52 (47-58) |
| 67-90% | 78 (77-80) | 59 (57-62) |
| 34-66% | 81 (80-82) | 66 (64-67) |
| 0-33% | 83 (82-84) | 70 (68-71) |
| Smoking | | |
| Current | 76 (75-77) | 60 (58-61) |
| Previous | 82 (81-83) | 61 (59-63) |
| Never | 87 (86-88) | 76 (74-78) |

#Based on 8899 participants from The Copenhagen City Heart Study 1991-1994 examination followed for 16 years.

TABLE 4
Baseline characteristics of study participants from the general population

[symbol]

| | Categories by sex and 10-year age | | | | | |
|---|---|---|---|---|---|---|
| | plasma YKL-40 percentile | | | | | |
| Characteristics | 0-33% | 34-66% | 67-90% | 91-95% | 96-100% | P Trend |
| Number (%) | 2964 (33) | 2932 (33) | 2121 (24) | 445 (5) | 437 (5) | — |
| Women, % | 57 | 56 | 56 | 56 | 57 | 0.96 |
| Age, years | 61 (48-71) | 61 (48-71) | 61 (48-71) | 60 (48-71) | 61 (48-71) | 0.12 |
| Current smokers, % | 43 | 48 | 51 | 56 | 58 | 0.0005 |

[symbol]

Values were collected at the 1991 through 1994 examination of the Copenhagen City Heart Study, and expressed as number, percent, or median (inter-quartile range).
Statistical comparisons between the five YKL-40 percentile categories were made using trend test (YKL-40 categories were coded 0, 1, 2, 3, and 4 for increasing percentile categories).

### Example 2

Diurnal, Weekly and Long Time Variation in Serum Concentrations of YKL-40 in Healthy Subjects

Materials and Methods

Reference Interval

Serum was collected from 245 healthy subjects (women/men 134/111, median age 49 years, range 18-79).

Diurnal Variation

Serum was collected seven times during a 24 hour period (day 1: 10 AM, 1 PM, 4 PM, 7 PM, 10 PM; day 2: 7 AM, 10 AM) from 16 healthy subjects (10/6, 48 years, range 32-66).

Day-to-Day Variation over 3 Weeks

Serum was collected at 8 AM five times during a 3 week period (day 1, 2, 8, 15, and 22) from 38 subjects recruited from the hospital staff (21/17, 41 years, range 22-66). At day 8 samples were also collected at 2 PM.

Week-to-Week Variation over 2 Years

Serum was collected from 23 subjects recruited from the hospital staff (14/9, 42 years, range 31-66) at 8 AM five times during a 3 week period (day 1, 2, 8, 15, and 22) and repeated 6, 12 and 24 months later.

Variation over 3 Years

Serum was collected between 8 AM and 10 AM five times during a 4 week period (day 1, 8, 15, 22 and 29) from 30 healthy women (48 years, range 24-62), and repeated 3 years later in 21 of the subjects.

Variation after Exercise

Serum was collected before physical exercise, immediately after a biphasic 25 minutes exercise program using an ergometer bicycle, and 1 and 3 hours post-exercise from 14 healthy subjects (10/4, 50 years, range 35-64). The healthy subjects included in the present study had no previous medical history, did not experience any symptoms and had no signs of disease and were not taking any medicine.

Ethics

The studies were approved by the regional scientific ethical committee and carried out in accordance with the Declaration of Helsinki. The subjects were informed about the studies verbally and in writing and all gave their written informed consent. All were informed that they could stop the study at any time.

YKL-40 ELISA

Proper handling of blood samples are important to minimize changes in serum YKL-40 that are not related to disease processes but represent metodological variability (Johansen et al., 2006, A; Johansen et al., 2006, B; and Harvey et al., 1998). Blood samples were allowed to clot at room temperature, centrifuged within ½-2 hours at minimum 2500 g for 10 minutes and serum was stored at −80° C. until analysis. Serum YKL-40 was determined in duplicates by a commercial two-site, sandwich-type enzyme-linked immunoassay (ELISA) (Quidel Corporation, San Diego, Calif.) using streptavidin-coated microplate wells, a biotinylated-Fab monoclonal capture antibody, and an alkaline phosphatase-labeled polyclonal detection antibody (Harvey et al., 1998). The recovery of the ELISA was 102% and detection limit 20 μg/L (Johansen et al., 2006, B; and Harvey et al., 1998). The intra-assay coefficient of variation (CV) was ≦5.0% and inter-assay CVs ≦0.2% (personal observation). Samples from each subject were analyzed on the same ELISA plate.

Statistical Analysis

Descriptive statistics for serum YKL-40 were presented by the median or the geometric mean, coefficient of variation and 95% confidence interval and range. The distribution of serum YKL-40 is skewed and therefore the log transform (natural) is used for statistical estimation. The reference interval was estimated using linear regression with YKL-40 on the log scale. The variations in serum YKL-40 analysed over time (variability during 24 hours, over 3 weeks, 6 months, 12 months, 24 months and 3 years) were given by the CV and compared to the intra- and inter-assay CV of the YKL-40 ELISA. The variance components for within subjects, between subjects and between rounds were estimated assuming a random effects model with YKL-40 log transformed (multiplicative model) and presented by the coefficient of variation of the geometric means (Kirkwood, 1979). The 95% confidence limits for the difference between 2 measurements of YKL-40 in an individual were calculated on the log scale and back transformed. The relative homogeneity between subjects compared to the total variation was estimated by the intraclass correlation coefficient. Serum YKL-40 in the analysis of diurnal long term variation and physical activity were analysed using a general linear model with repeated measures. P-values <5% were considered significant. P-values for multiple testing were corrected using the Boneferroni correction. All statistical calculations were done using SAS (9.1, SAS Institute, Cary, N.C., USA).

Results

In healthy subjects the median serum YKL-40 was 43 μg/l (range: 20-184 μg/L; 5-95% interval: 20-124), and no difference between men and women (P=0.54). Serum YKL-40 increased with age (rho=0.45; P<0.0001). A normal reference interval for serum YKL-40 adjusted for age and gender was constructed by linear regression with serum YKL-40 as the dependent variable (log transformed) and age and gender as the explanatory variables. The upper limit was defined as the 95th percentile for given age and gender. The inter subject CV adjusted for age was 45%.

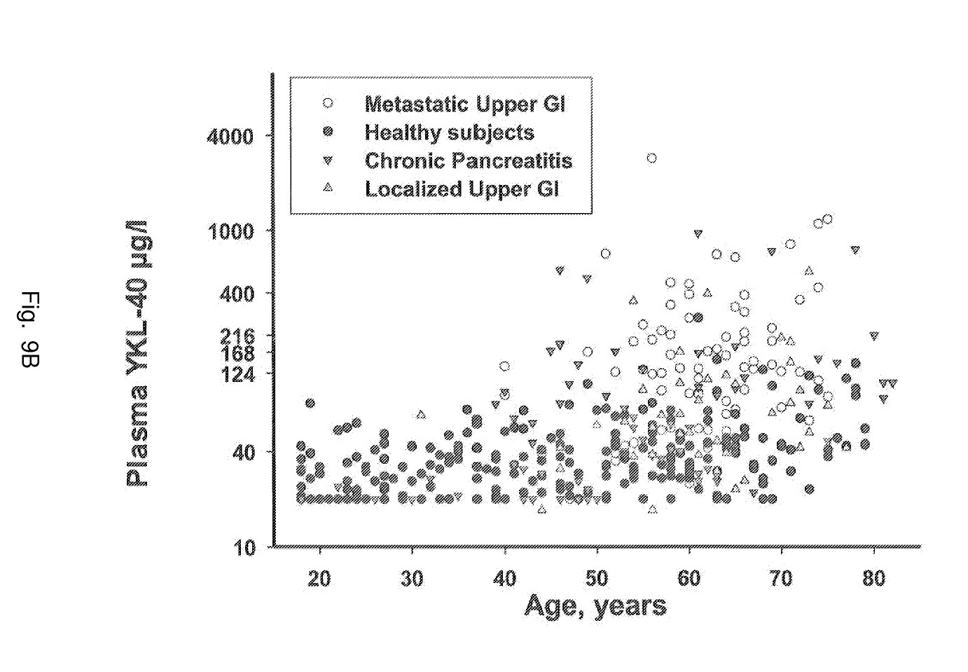

FIG. 5 illustrates the individual diurnal variation in serum YKL-40 at 7 time points during 24 hours. The mean serum YKL-40 increased 23% from 10 AM to 10 PM (P=0.01), however nonsignificant when corrected for multiple testing. No other significant differences were observed.

No changes in serum YKL-40 were found after 25 minutes of bicycling (P>0.08, linear model).

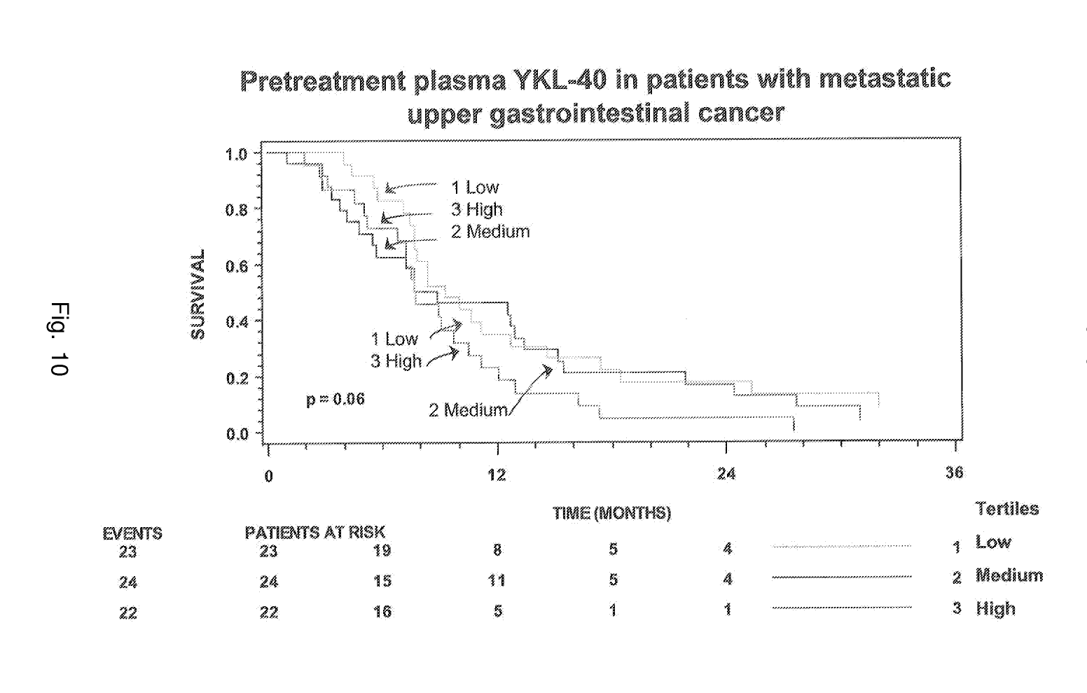

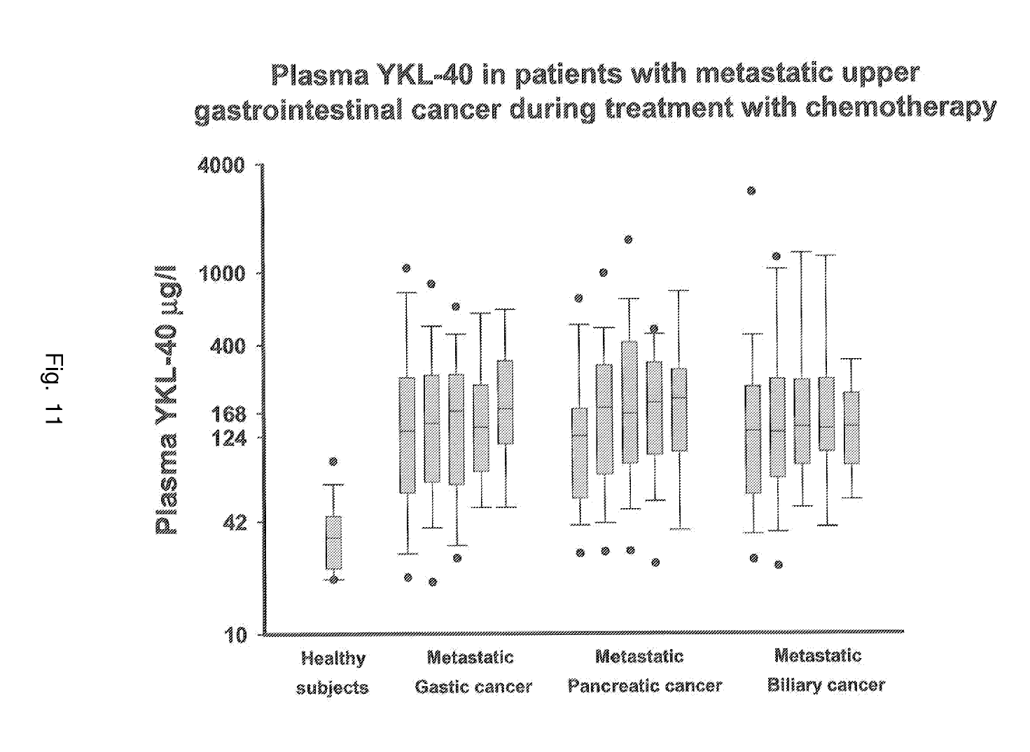

FIG. 6 shows the individual weekly changes in serum YKL-40 at 6 time points during a 3 weeks period (at 8 AM on day 1, 2, 8, 15 and 22). The median day to day CV of serum YKL-40 for each subject was 16%. On day 8 samples were collected at 8 AM and 2 PM and serum YKL-40 increased slightly (47 μg/L vs. 52, 8% difference, P<0.0001).

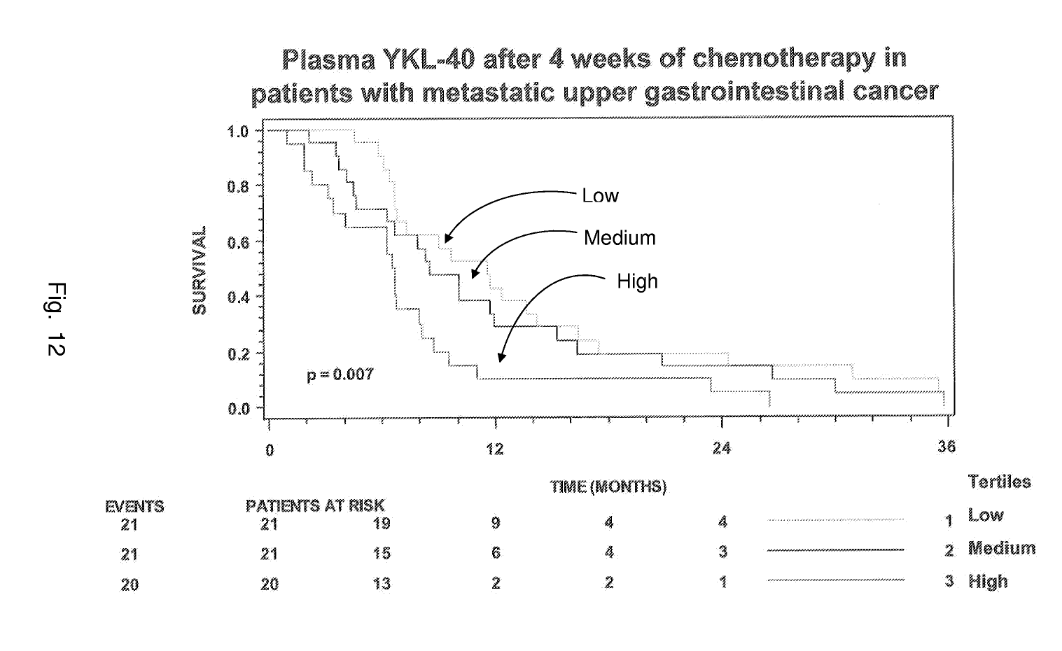

FIG. 7 illustrates the individual variation in serum YKL-40 at five time points during a 3 week period (at 8 AM on day 1, 2, 8, 15 and 22, 1st round) and repeated after 6 months (2nd round), 12 months (3rd round) and 24 months (4th round). The median day to day CV of serum YKL-40 for each subject was overall 16% (range 0-92%), and 16% (0-63%, 1st round), 19% (5-92%, 2nd), 15% (0-64%, 3rd), and 21% (0-47%, 4th).

No systematic increases or decreases were detected over the 4 rounds (P=0.09). The estimates of the variance components using a random effects model with serum YKL-40 log transformed results in a within subject CV of 27.3% and a CV over 24 months of 8.8%. The within subject CV including the variation over time and inter-assay variation was 30.2% over the 24 months period. The intraclass correlation coefficient over the 24 months was 72.4%. The estimated variation in serum YKL-40 within subjects including inter-assay variation results in 95% confidence limits for the difference between two measurements on the same subject if the second YKL-40 measurement is reduced by 52% or is increased by 109% and differences of this magnitude are significant and not only a reflection of pre-analytical conditions, methodological and normal biologic variability.

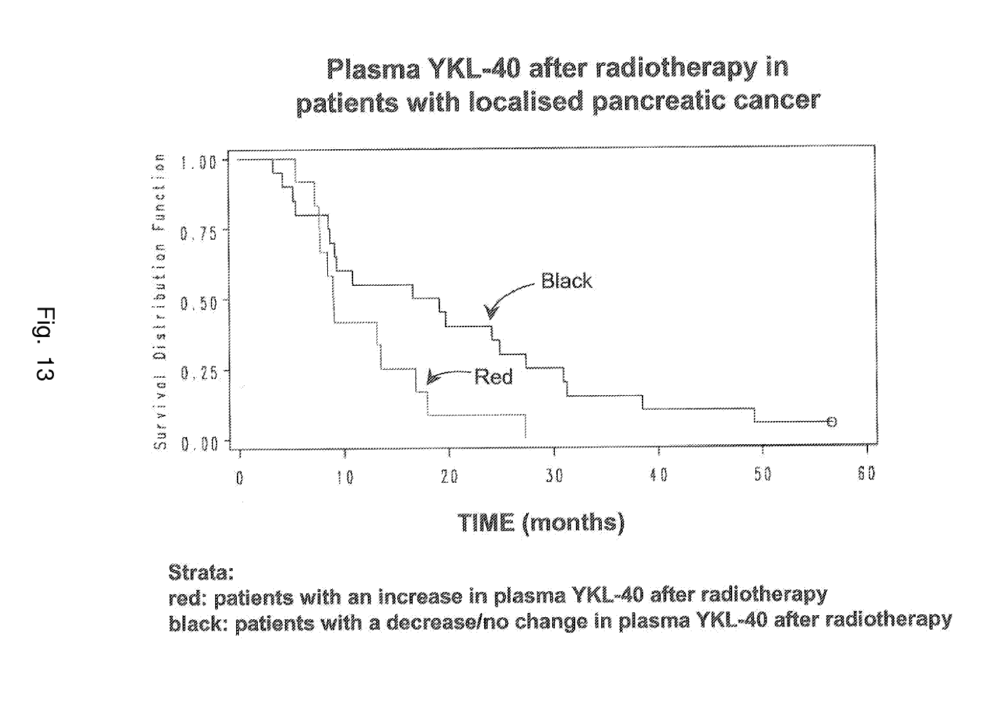

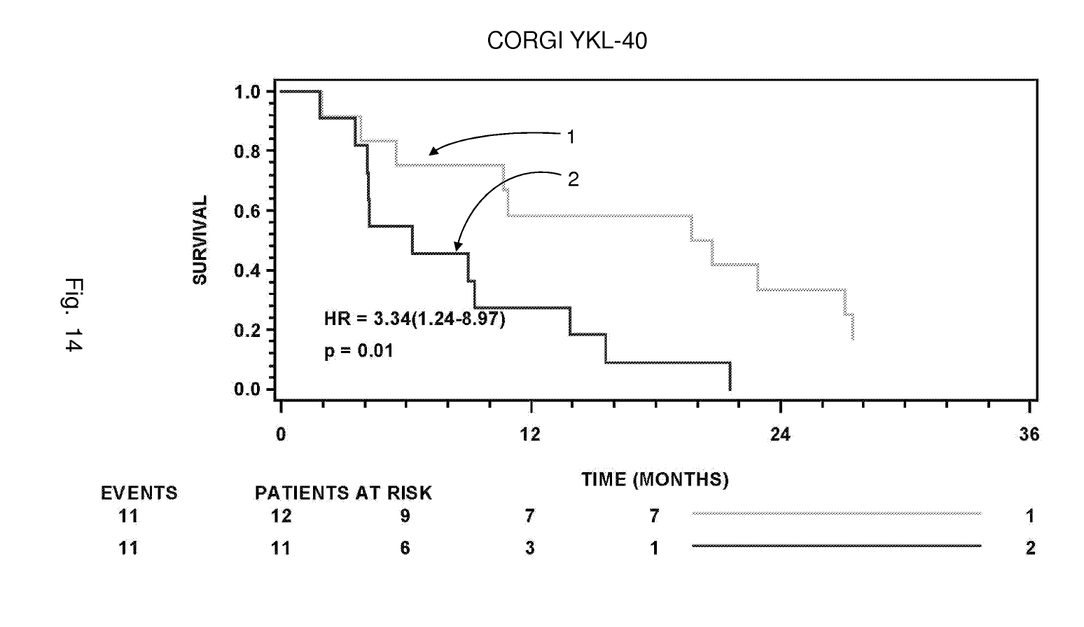

FIG. 8 shows the individual weekly changes in serum YKL-40 at five time points during a month and subsequently again after 3 years. The median CV in serum YKL-40 was 17% (1st round) and 13% (2nd round). In subjects analyzed in both rounds (n=21) no changes in serum YKL-40 were observed between the two periods (P=0.37, linear model). The estimates of the variance components using the random effects model with serum YKL-40 log transformed result in a within subject CV of 26.0% and CV over 3 years of 7.3%. The within subject CV including the variation over time and inter-assay variation was 28.8%. The between subject variation including within subject variation and variation over time was 54%. The intraclass correlation coefficient over 3 years was 72.2% suggesting a relatively low within subject variation compared to between subject variation.

Conclusions

The present study demonstrates that serum YKL-40 is stable in healthy subjects for short term as well as long term sampling periods of up to 3 years with a within subject CV of ˜30% including inter-assay variation. The between subject variation in serum YKL-40 was 45% in the study determining a normal reference interval and similar to that found in the other studies of healthy subjects in the present study.

The intraclass correlations of serum YKL-40 were 72.4% and 72.2% over a period of 2 and 3 years, suggesting a relative low within subject variation compared to between subject variations. The intraclass correlations found in the present study are similar to those found for other serological markers, for example Ockene et al. reported an intraclass correlation of 66% for high sensitive C-reactive-protein (Ockene et al., 2001).

The present estimated variation in serum YKL-40 within healthy subjects including inter-assay variation determined that an increase of >109% or a decrease of >52% in serum YKL-40 is considered as significant and not only a reflection of pre-analytical conditions, methodological and normal biologic variability.

In conclusion, the present study showed that there are no significant diurnal variation in serum YKL-40 nor an effect of physical exercise. A relatively low within subject variation compared to between subject variation in serum YKL-40 was demonstrated confirming that YKL-40 is a reliable biomarker.

### Example 3

Upper GI Cancer—Prognostic and Predictive Value of YKL-40

The purpose of the present study was to investigate in patients with upper gastrointestinal cancer the prognostic and predictive value of plasma concentrations of YKL-40 and IL-6 treated with chemo/radiotherapy for localized disease or chemotherapy for metastatic disease.

Patients and Methods

Study Populations

CORGI Study: Forty patients with localized upper GI-cancers were included in a longitudinal study of the effect of chemo/radiotherapy. Plasma samples were collected before, after 2 cycles of Xelox (oxaliplatin 130 mg/m² iv on day 1 and capecitabine 1000 mg/m² twice daily po on days 1-14 and, every 3. week). The patients were then treated with radiotherapy (50.4 Gy in 1.8 Gy fractions) to gross tumour volume in combination with a reduced Xelox regimen (oxaliplatin 30-60 mg/m² iv on day 1 and capecitapin 675-750 mg/m² twice daily p.o. every day of radiotherapy). In patients with gastric and pancreatic cancer radiotherapy was also give to adjacent lymph nodes (41.4 Gy in 1.8 Gy fractions). Plasma samples were collected 4-6 weeks after the end of chemoradiotherapy.

GITAC Study: Seventy patients with metastatic upper GI-cancers were included in a longitudinal study of the effect of sequential treatment with docetaxel 45 mg/m² or irinotecan 180 mg/m² every second week together with 5-FU/leucovorin (500 mg/m²+60 mg/m²×2, Nordic schedule, except patients with gastric carcinomas, who were treated with de Gramont schedule). During treatment with chemotherapy plasma samples were collected after 2 weeks, 4 weeks, 6 weeks and 8 weeks.

YKL-40 Analysis

Plasma concentrations of YKL-40 were measured by a two-side, sandwich-type Elisa (Quidel, Calif., USA) in accordance with the manufacturer's instructions. The sensitivity was 20 μg/land the intra- and inter-assay coefficient of variations were ≦5.0% and ≦8.4%. To eliminate the inter-assay variation samples from each patient were analyzed in the same assay. ELISA kits with the same batch number were used for all patients.

Plasma YKL-40 in Healthy Subjects

The reference intervals for plasma YKL-40 were determined in 234 healthy subjects characterized by not being on medication and having no signs of pre-existing disorders such as joint, liver, metabolic or endocrine disease or malignancy (38).

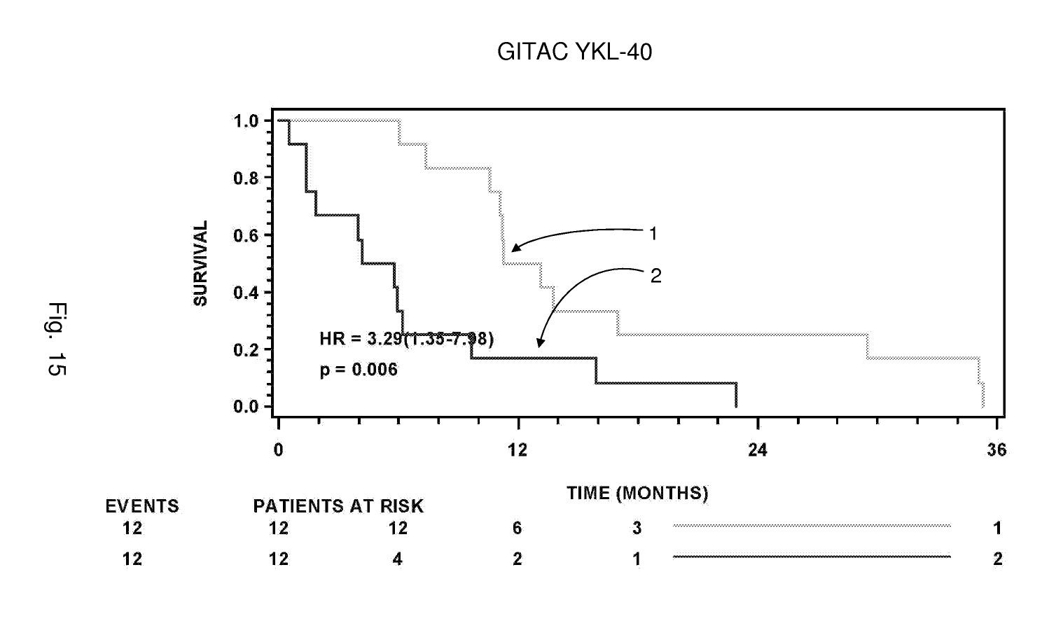

Statistical Analysis I—Basis for FIGS. 9A, 9B, 10, 11, 12, 13 and Gable 5

The clinical endpoints for this biomarker study were overall survival determined as the time from baseline blood sample before chemotherapy to time of death of all causes. All data on disease status and duration of survival were updated in 2008, where all patients were dead. Plasma concentrations of YKL-40 were considered both at baseline and after first, second, third and fourth treatment. Kruskal-Wallis test was used for comparison of three or more independent groups with nonparametric data distributions. Survival probabilities for overall survival were estimated by the Kaplan-Meier method and tests for differences between strata were done using the log-rank statistic. Graphical presentation of plasma YKL-40 levels using Kaplan-Meier estimates of survival were shown grouping patients by tertiles (normal, slightly/moderate elevated, highly elevated). Analyses of overall survival for continuous covariates as well as multivariate analyses were done using the Cox proportional hazards model. Plasma YKL-40 were entered by the actual value on the log scale (base 2). Model assessment was done using graphical methods. Analyses of updated levels of plasma YKL-40 during treatment were done using time-dependent a Cox proportional hazards model. P-values less than 5% were considered significant. All calculations were performed using SAS (version 9.1, SAS Institute, Cary, N.C., USA).

Statistical Analysis II—Basis for FIGS. 14 and 15

The clinical endpoint for this biomarker study were overall survival determined as the time from baseline blood sample before chemotherapy to time of death of all causes. All data on disease status and duration of survival were updated in September 2008 (CORGI Study) and in January 2008 (GITAC Study). Plasma YKL-40 and IL-6 were considered both at baseline and during treatment. Descriptive statistics for plasma YKL-40 and IL-6 are presented by their median levels and the range. Rank statistics were used for tests for location and performance status (Wilcoxon rank sum) and measures of association (Spearman rank correlation). Analyses of overall survival for continuous covariates as well as multivariate analyses were done using the Cox proportional hazards model. Plasma YKL-40 and IL-6 at baseline were entered by the actual value on the log scale (base 2). For analysis of survival at 4-6 weeks after end of radiochemotherapy were done using the landmark method for the CORGI Study, and for analysis of survival at 2, 4 and 6 weeks after start of chemotherapy were done using the landmark method for the GITAC Study. The ratios of the plasma YKL-40 and IL-6 levels to baseline levels were used for analysis of longitudinal data. Model assessment was done using graphical methods. Survival probabilities for overall survival were estimated by the Kaplan-Meier method and tests for differences between strata were done using the log-rank statistic. Patients were dichotomized by the median ratios of plasma YKL-40 and IL-6 compared to baseline levels. P-values less than 5% were considered significant. All calculations were performed using SAS (version 9.1, SAS Institute, Cary, N.C., USA).

Results

Pretreatment YKL-40 of the Patients

The baseline median plasma YKL-40 concentrations in the patients with localized upper GI-cancer plasma YKL-40 was higher (p<0.001) (median 64 μg/l, range 20-545) compared to healthy subjects (34 μg/l, 20-258) (Table 5.). The baseline median plasma YKL-40 concentrations of the patients with metastatic upper GI-cancer was higher (p<0.001) in the patients (median 127 μg/l, range 20-2869) compared to healthy subjects (34 μg/l, 20-258) (Table 5.). Plasma YKL-40 was higher than the upper normal level (i.e. defined as the age corrected upper 95% percentile in healthy subjects) in 33% of the patients with localized pancreatic cancer, in 50% of the patients with localized biliary or gastric cancer, in 81% of the patients with metastatic pancreatic cancer, in 85% with metastatic billiary cancer and in 77% with metastatic gastric cancer (Table 5.).

TABLE 5
Pre-treatment concentrations of plasma YKL-40 in 40 patients with
localized upper GI cancer and in 70 patients with metastatic upper
GI cancer

| | Localized Cancer | | | | |
|---|---|---|---|---|---|
| Charac- | | Gastric or | Metastatic Cancer | | |
| teristic | Pancreatic | Billiary | Pancreatic | Gastric | Biliary |
| Number | 30 | 10 | 27 | 22 | 21 |
| Plasma | 54 | 73 | 124 | 133 | 132 |
| YKL-40 | (20-545) | (20-396) | (20-710) | (20-1097) | (25-2869) |
| μg/l# | | | | | |
| Elevated | 10 | 5 | 22 | 17 | 17 |
| YKL-40 [symbol] | (33%) | (50%) | (81%) | (77%) | (85%) |

#Values are median (range) [symbol]
Number of patients with elevated YKL-40 (%) compared to age-matched healthy subjects (i.e. an YKL-40 value higher than the 95% percentile)

FIG. 9A illustrates the individual plasma YKL-40 levels according to age and type of cancer in patients with metastatic upper gastrointestinal cancer. For comparison plasma YKL-40 levels in healthy subjects are also included.

FIG. 9B illustrates the individual plasma YKL-40 levels in patients with localized upper gastrointestinal cancer, in patients with metastatic upper gastrointestinal cancer, and in patients with chronic pancreatitis. For comparison plasma YKL-40 levels in healthy subjects are also included.

Pretreatment plasma YKL-40 was not associated with performance status (p=0.08) and not correlated with serum CA 19-9 (p=0.39) and CEA (p=0.78) in patients with metastatic upper gastrointestinal cancer.

Pretreatment Plasma YKL-40 and Overall Survival—with Basis in Statistical Analysis I

In patients with localized upper GI cancer pretreatment plasma YKL-40 levels (log transformed, treated as a continuous covariate) showed that YKL-40 was not associated to overall survival (HR=0.80, 95% Cl 0.51-1.24, p=0.31)

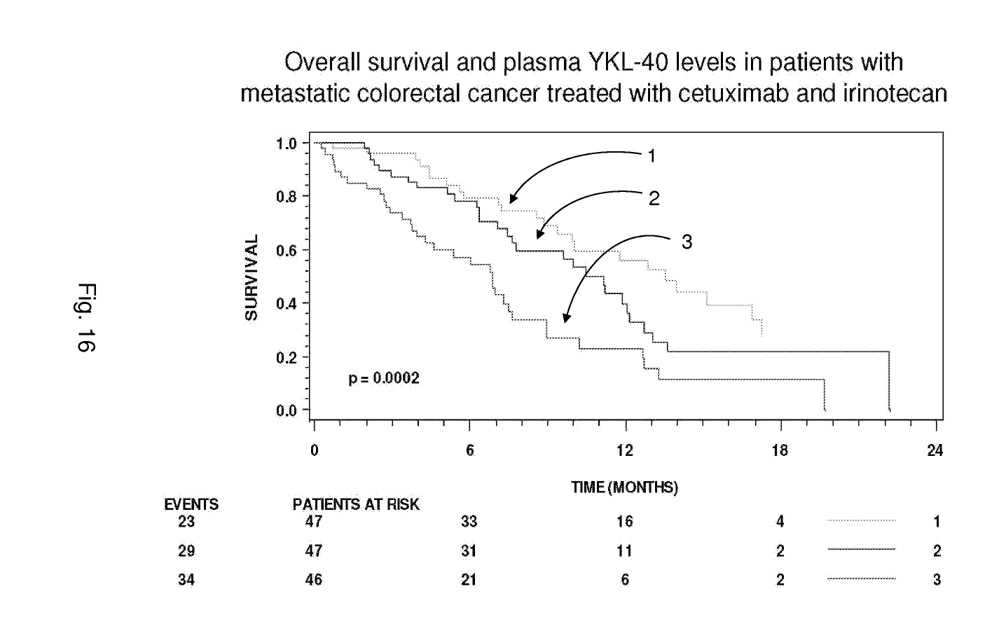

At time of follow-up all patients with metastatic upper GI had died. The median survival time was 8.6 months (range 1-38). The Kaplan-Meier estimates of survival stratified by pre-treatment plasma YKL-40 (dichotomized in tertiles) are shown in FIG. 10. Univariate analysis of pretreatment plasma YKL-40 (log transformed, treated as a continuous covariate), stratified by diagnostic group, showed that pretreatment YKL-40 was not associated to overall survival in patients with metastic upper GI (HR=1.21, 95% Cl: 0.93-1.58, p=0.15) and progression free survival (HR=1.12, 95% Cl: 0.87-1.46, p=0.35).

Plasma YKL-40 During Follow-Up and Prediction of Overall Survival—with Basis in Statistical Analysis I

Samples were obtained from the patients with localized upper GI after radiotherapy. Univariate analysis of plasma YKL-40 levels after end of radiotherapy (defined as the ratio of plasma YKL-40 =concentration at the end of radiotherapy compared to the baseline level) showed that an increase of plasma YKL-40 was associated to short overall survival in patients with localized upper GI (HR=2.42, 95% Cl: 1.16-5.04, p=0.019). The corresponding Kaplan-Meier estimates of survival are shown in FIG. 13. Only patients with localized pancreatic cancer are included in this analysis.

Samples were obtained from the patients with metastatic upper GI after chemotherapy.

During treatment plasma YKL-40 increased in patients with metastatic pancreatic cancer (p<0.01) and was unchanged in patients with gastric cancer and biliary cancer (FIG. 11). The Kaplan-Meier estimates of survival stratified by plasma YKL-40 after 4 weeks of radiotherapy treatment (dichotomized in tertiles, landmark test) are shown in FIG. 12. Patients with high plasma YKL-40 4 weeks after treatment had significantly shorter survival than patients with normal plasma YKL-40 (p=0.007, log-rank test).

Multivariate analysis including diagnostic group, age, performance status, and plasma YKL-40 after 4 weeks of treatment showed that YKL-40 was significant in predicting overall survival (HR=1.54, 1.08-2.19, p=0.017) and time to progression (HR=1.46, 1.01-2.02, p=0.04).

Pretreatment Plasma YKL-40 and Overall Survival—with Basis in Statistical Analysis II

CORGI Study. At time of follow-up one patient was still alive. The median survival time was 12.0 months (95% Cl 9.0-16.8). Univariate analysis of pretreatment plasma YKL-40 (log transformed, continuous covariate) in patients with pancreatic cancer showed that pretreatment YKL-40 was not associated to overall survival (HR=0.86, 95% Cl 0.63-1.16, p=0.32).

GITAC Study. At time of follow-up all patients had died. The median survival time was 8.4 months (range 1-38, 95% Cl 7.7-10.7). Univariate analysis of pretreatment plasma YKL-40 (log transformed, continuous covariate) showed that pretreatment YKL-40 was not associated to overall survival in patients with pancreatic cancer (HR=1.16, 95% Cl 0.84-1.62, p=0.36), gastric cancer (HR=1.12, 0.85-1.48, p=0.43) and biliary cancer (HR=1.07, 0.74-1.55, p=0.72).

Plasma YKL-40 During Treatment and Follow-Up and Prediction of Death—with Basis in Statistical Analysis II

CORGI Study. After 2 cycles of Xelox and just before start of radiochemotherapy plasma YKL-40 increased in 23 (85%) of the patients with pancreatic cancer. 4-6 weeks after the end of radiochemotherapy 10 (42%) of the patients had lower plasma YKL-40 compared to pretreatment levels. Univariate analysis of plasma YKL-40 in pancreatic cancer patients 4-6 weeks after end of radiochemotherapy (ratio compared to baseline value, continuous variable) showed that high YKL-40 ratio was associated with short overall survival (HR=3.27, 1.40-7.63, p=0.006). The corresponding Kaplain-Meier estimates of survival 4-6 weeks after the end of radiochemotherapy are shown in FIG. 14. Multivariate analysis (PS, YKL-40 and IL-6, continuous variables) showed that the actual value of plasma YKL-40 4-6 weeks after end of treatment was an independent biomarker of short survival (HR=2.91, 1.09-7.75, p=0.032).

GITAC Study. During treatment plasma YKL-40 increased compared to baseline in patients with pancreatic cancer (YKL-40: 2 weeks p=0.006, 4 weeks p=0.0002 and 6 weeks p=0.0002). In patients with pancreatic cancer univariate analysis of plasma YKL-40 ratios 2, 4 and 6 weeks after start of chemotherapy (ratio compared to baseline value, continuous variable) showed that high YKL-40 ratio after 4 weeks was associated with short overall survival (HR=1.35, 1.06-1.72, p=0.017). The corresponding Kaplain-Meier estimates of YKL-40 ratios 4 weeks after start of chemotherapy are shown in FIG. 15.

The actual plasma YKL-40 values (log transformed) were significant in univariate analysis in pancreatic cancer patients for week 4 (YKL-40: HR=1.50, 1.06-2.13, p=0.023).

Conclusion

In the present study we found that 38% of patients with localized upper GI cancer and 81% with metastatic upper GI cancer had elevated plasma YKL-40 at time of diagnosis. These numbers are higher compared to other types of adenocarcinomas, and may reflect the very poor prognosis of patients with upper GI cancer. Interestingly, patients with localized pancreatic cancer and no change or a decrease, compared to baseline level, in plasma YKL-40 four to six weeks after the end of radiochemotherapy had a better survival compared to patients with an increase in plasma YKL-40. Similarly results were found in patients with metastatic pancreatic cancer for the ratio in plasma YKL-40 four weeks after start of chemotherapy. These are all novel observations and suggest that changes in plasma YKL-40 during or after treatment are useful biomarkers to monitor in patients.

### Example 4

High Pretreatment Plasma YKL-40 Levels in Patients with Metastatic Colorectal Cancer Treated with Cetuximab are Associated with Short Survival

Patients

Prospective, longitudinal study of 140 patients (median age 63 years, range 36-87 years, performance status 0-2) with metastatic colorectal cancer resistant to 5-FU, oxaliplatin and irinotecan. The patients were then treated with irinotecan (130 mg/m²) and cetuximab (500 mg/m²) every second week independent of their KRAS status. Median follow-up time was 15 months (range 2.5-25 months). 86 patients died. Plasma YKL-40 was analyzed by ELISA (Quidel). KRAS was analyzed using DxS KRAS test kit (Roche).

Results

The median overall survival was 9.6 months. KRAS status was analyzed in 86 (61%) patients (wild type n=47, mutated n=39). Overall survival in patients with KRAS wild type was 12.1 months compared to 7.0 months in patients with KRAS mutations (p=0.08).

Pretreatment plasma YKL-40 (median 131 μg/l, range 15-1766) was elevated (i.e. >95^{th} percentile in healthy subjects, age-corrected level) in 66% of the patients. Plasma YKL-40 was not associated with KRAS status (p=0.39). YKL-40 correlated with CEA (r=0.32, p=0.0004).

Univariate analysis (log transformed continuous variable (base 2)), showed that high pretreatment plasma YKL-40 was associated with short overall survival (HR=1.29, 95% Cl: 1.12-1.49, p=0.0006). From this analysis patients with plasma YKL-40 levels 67 μg/l (first quartile), 131 μg/l (median) and 259 μg/l (third quartile) had 8 months survival of 62% (95% Cl: 52-72), 54% (95% Cl: 45-64) and 45% (95% Cl: 36-56), respectively. The Kaplan-Meier curves for these 3 groups for overall survival are illustrated in FIG. 16.

Multivariate Cox analysis (plasma YKL-40, age, sex, performance status, serum CEA) showed that pretreatment YKL-40 (HR=1.20, 95% Cl: 1.03-1.40, p=0.03) and performance status (0 vs. 1: 1.71, 0.99-2.94; 0 vs. 2: 3.62, 1.98-7.03, p=0.001) were independent factors of overall survival. Serum CEA (p=0.30) and KRAS status (p=0.13) were not significant in this model.

Conclusion

High pretreatment plasma YKL-40 was an independent prognostic biomarker of short overall survival in patients with metastatic colorectal cancer treated with cetuximab in combination with irinotecan. Thus plasma YKL-40 may be a new predictive biomarker of response to cetuximab, and thus a biomarker for selection of treatment for a specific disease.

### Example 5

High Pretreatment Plasma and Serum Concentrations of YKL-40 in Patients with Metastatic Colorectal Cancer Treated with Irinotecan and Cetuximab are Associated with Short Overall Survival and Short Progression Free Survival and is Independent of KRAS

Patients

Study 1: Prospective, longitudinal study of 196 patients with metastatic colorectal cancer resistant to 5-FU, oxaliplatin and irinotecan. The patients were treated with third-line irinotecan (130 mg/m² of body-surface area on day 1 of each 14-day period during the study) and cetuximab (first dose 400 mg/m² of body-surface area, then at a dose of 500 mg/m² of body-surface area every second week independent of their KRAS status). The patients were treated until disease progression. Median follow-up time was 19 months (range 6-31 months). 148 patients died. This study is a continuation of Example 4 herein, now including the entire group of patients.

Study 2: Retrospective, longitudinal study of 134 patients with metastatic colorectal cancer resistant to 5-FU, oxaliplatin and irinotecan. The patients were treated with third-line irinotecan (130 mg/m² of body-surface area on day 1 of each 14-day period during the study) and cetuximab (first dose 400 mg/m² of body-surface area, then at a dose of 250 mg/m² of body-surface area once weekly independent of their KRAS status). The patients were treated until disease progression. Median follow-up time was 30 months (range 14-50 months). 98 patients died.

Methods

Pretreatment plasma was available for YKL-40 analysis from 185 of the patients included in Study 1. Pretreatment serum was available for YKL-40 analysis from 134 patients included in Study 2. Plasma concentrations of YKL-40 (Study 1) and serum concentrations of YKL-40 (Study 2) were analyzed by a commercial ELISA (Quidel, Calif., USA).

DNA from primary tumor was available for KRAS mutation status from 180 of the patients included in Study 1 and from 99 patients included in Study 2. KRAS was analyzed using DxS KRAS test PCR kit (Roche).

Statistical Analysis

The primary clinical endpoint for this study was overall survival determined as the time from baseline blood sample before start of treatment with cetuximab to time to death of all causes. All data on disease status and duration of survival were updated Jul. 2, 2009 (Study 1) and Mar. 9, 2009 (Study 2). Cases in which patients were alive by this date were censored. Secondary endpoint was time to disease progression (only Study 2).

Plasma or serum concentrations of YKL-40 were determined at baseline, prior to first treatment with cetuximab. Different cut-off levels of plasma YKL-40 (Study 1) and serum YKL-40 (Study 2) in healthy subjects (age-corrected) were chosen: The 90, 95, 97.5, 99, 99.5 and 99.9 percentile levels. Plasma and serum YKL-40 levels of the two patient groups were also divided into tertiles and used as cut-off levels. Descriptive statistics are presented by their median levels and range. Rank statistics were used for tests of association between plasma and serum YKL-40 with KRAS and performance status (Wilcoxon rank sum) and measures of association (Spearman rank correlation).

Kruskal-Wallis test was used for comparison of three or more independent groups with nonparametric data distributions. Analysis of measurements for time to disease progression and death were done using the Cox proportional hazards model. Plasma and serum levels of YKL-40 were entered by their actual value (log transformed) on the log scale (base 2) or by high vs. normal level (the 95 percentile in healthy subjects was used as cut-off). Only cases with complete data were included in the multivariate analyses. Analysis of response to cetuximab was done using logistic regression and presenting the results using odds ratios (OR) with 95% confidence limits (Cl) as well as the area (AUC) under the receiver operating characteristic curve (ROC). Model assessment was done using graphical methods. Survival probabilities for overall survival were estimated by the Kaplan-Meier method and tests for differences between strata were done using the log-rank statistic. Graphical presentation using Kaplan-Meier estimates of survival was shown grouping patients by their tertiles of plasma and serum YKL-40 levels or the following cut-off levels of age-corrected YKL-40 levels in healthy subjects: 90%, 95%, 97.5%, 99%, 99.5%, and 99.9%. Model assessment was done using graphical methods, Schoenfeld and martingale residuals. P-values less than 5% were considered significant. All calculations were performed using SAS (version 9.1, SAS Institute, Cary, N.C., USA).

Results

Pretreatment Plasma and Serum YKL-40 Levels and Demographic Characteristics of the Patients

The baseline demographic characteristics of the patients with metastatic colorectal cancer included in Study 1 and Study 2 are shown in Table 6. The two study populations are comparable. 38% had KRAS mutations in Study 1 and 45% in Study 2. The patients had significantly (p<0.001) higher pretreatment plasma and serum YKL-40 levels compared to healthy subjects. Plasma and serum YKL-40 levels were higher than the upper normal level (95 percentile used as cut-off) in 52% of the patients in Study 1 and in 68% of the patients in Study 2. YKL-40 was not associated with KRAS status (Study 1: p=0.34; Study 2: p=0.45).

TABLE 6
Clinical characteristics of the patients and pretreatment
concentrations of plasma YKL-40 and serum YKL-40 in
patients with metastatic colorectal cancer treated
with irinotecan and cetuximab.

| | Study 1 | Study 2 | |
|---|---|---|---|
| Characteristic | N = 196 | N = 134 | P-value§ |
| Age, years | 64 (36-87) | 62 (38-82) | NS |
| Sex, male/female [symbol] % | 63%/37% | 54%/46% | NS |
| Metastatic sites, 1/2/3/4/ND | 104/53/17/1/21 | ND | ND |
| Number and percentages | 53%/27%/ | ND | |
| | 9%/0.5%/11% | | |
| Performance status, | 93/60/33/10 | 51/40/8/35 | NS |
| 0/1/2/ND | | | |
| Number and percentages | 47%/31%/ | 38%/30%/ | |
| | 17%/5% | 6%/26% | |
| KRAS mutations, | 69/111/16 | 45/54/35 | NS |
| MT/WT/ND | | | |
| Number and percentages# | 38%/62% | 45%/55% | |
| Plasma or serum | 133 (15-1766) | 148 (16-1410) | NS |
| YKL-40, μg/l | | | |
| Median (range) | | | |
| Patients with elevated | 97 (52%) | 91 (68%) | NS |
| YKL-40 | | | |
| Number (percentage) [symbol] | | | |

ND, not determined.
NS, not significant.
MT, KRAS mutations.
WT, KRAS wild type.
#Only the cohort with KRAS determinations. [symbol]
Only the cohort with YKL-40 determinations. The 95 percentile of plasma and serum YKL-40 levels in healthy subjects are used as cut-off (age-corrected).
§Mann-Whitney's test or Kruskal Wallis tests are used.

Pretreatment Serum YKL-40 Levels and Response to Cetuximab Therapy

Data are only available from Study 2: Twenty patients were classified as responders (all wild-type) and 76 as non-responders according to RECIST criteria (KRAS wild type: 33; KRAS mutated: 43). The corresponding serum YKL-40 levels in these 3 groups are shown in Table 7. Highest serum YKL-40 levels were found in patients with no response to treatment. Response is analyzed in the KRAS wild type group using logistic regression. The Odds ratio (OR) estimates are: serum YKL-40 entered by its actual value on the log scale (base 2): OR=1.34, 95% Cl: 0.89-2.01, p=0.16, AUC=0.61; and serum YKL-40 entered as its dichotomized level: OR=1.68, 95% Cl: 0.63-4.48, p=0.33. The fact that the 95% Cl's include 1 can likely be attributed to the small sample size.

Serum YKL-40 was independent of KRAS mutation status. High serum YKL-40 was associated with poor response to the Cetuximab treatment. Thus YKL-40 may be used to locate the group of true responders among the patients with KRAS wild type (20 out of 53, i.e. approximately 40% all KRAS wild type).

TABLE 7
Serum YKL-40 levels according to KRAS mutation status and
response in patients from Study 2.

| | | YKL-40, ug/l |
|---|---|---|
| KRAS Status | N | Median (range) |
| Wild type, response | 20 | 101 (44-639) |
| Wild type, no response | 33 | 159 (41-938) |
| Mutations, no response | 43 | 138 (16-1410) |

Pretreatment Serum YKL-40 Levels and Progression Free Survival

Data are only available from Study 2: Progression free survival was determined as time from date of first treatment and time to disease progression. 105 had progression.

Univariate Cox analysis showed that high pretreatment serum YKL-40 (log transformed continuous variable (base 2)) was associated with short progression free survival (HR=1.18, 95% Cl: 1.01-1.39, p=0.042). Multivariate Cox analysis (YKL-40 and KRAS) demonstrated that plasma YKL-40 was an independent biomarker of progression free survival (HR=1.20, 95% Cl: 1.02-1.41, p=0.026) and independent of KRAS status. The HR for YKL-40 is 1.20, i.e. the hazard increases by 20% for each doubling of YKL-40.

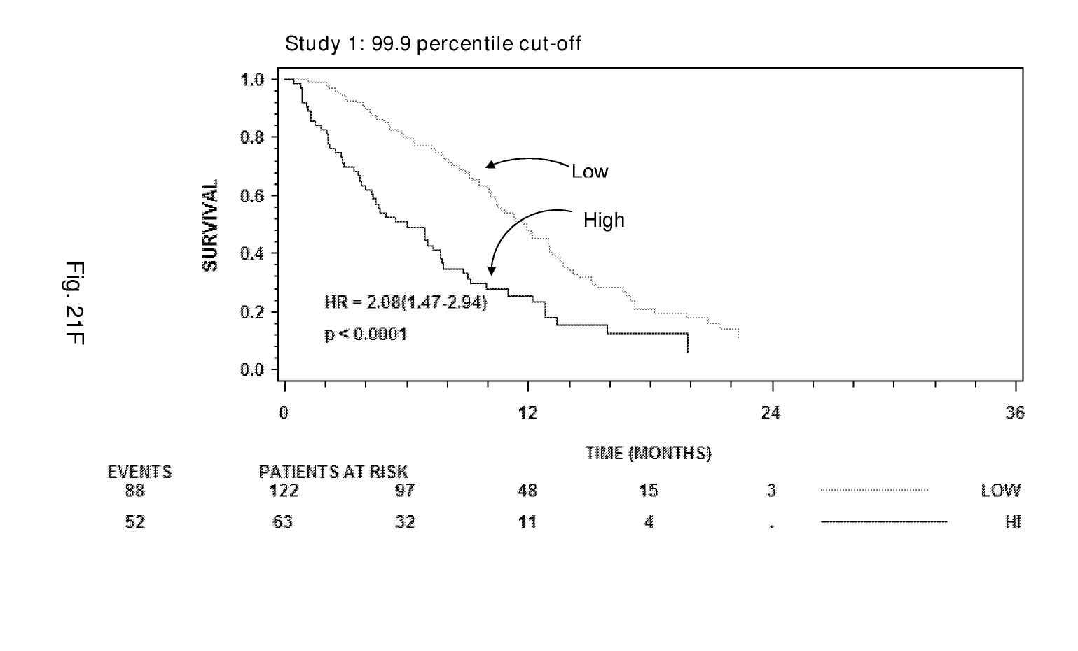

The Kaplan-Meier curves for increasing serum YKL-40 levels in the patients (tertiles are used as cut-off) and progression free survival are illustrated in FIG. 18. Significantly shorter survival was found according to increasing tertiles of pretreatment serum YKL-40.

Serum YKL-40 was independent of KRAS mutation status. High serum YKL-40 was associated with poor response to the Cetuximab treatment and short progression free survival. Thus YKL-40 may be used to locate the group of true responders among the patients with KRAS wild type (20 out of 53, i.e. approximately 40% all KRAS wild type).

Pretreatment Plasma and Serum YKL-40 Levels and Overall Survival

Study 1:

The median overall survival was 10.0 months. Overall survival in patients with KRAS wild type was 11.3 months compared to 7.5 months in patients with KRAS mutations (p=0.004).

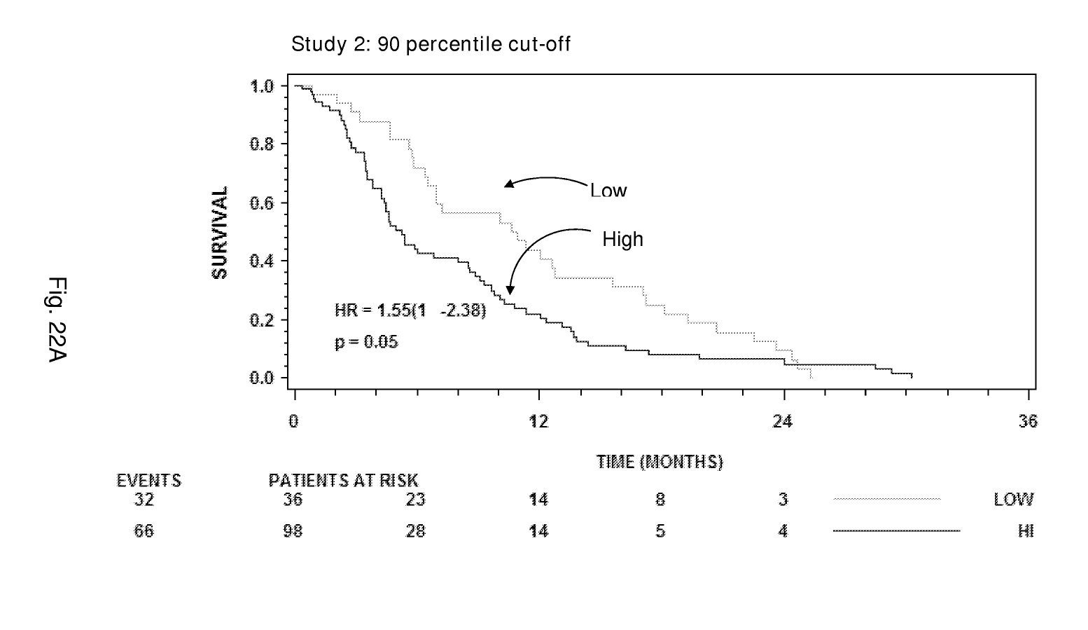

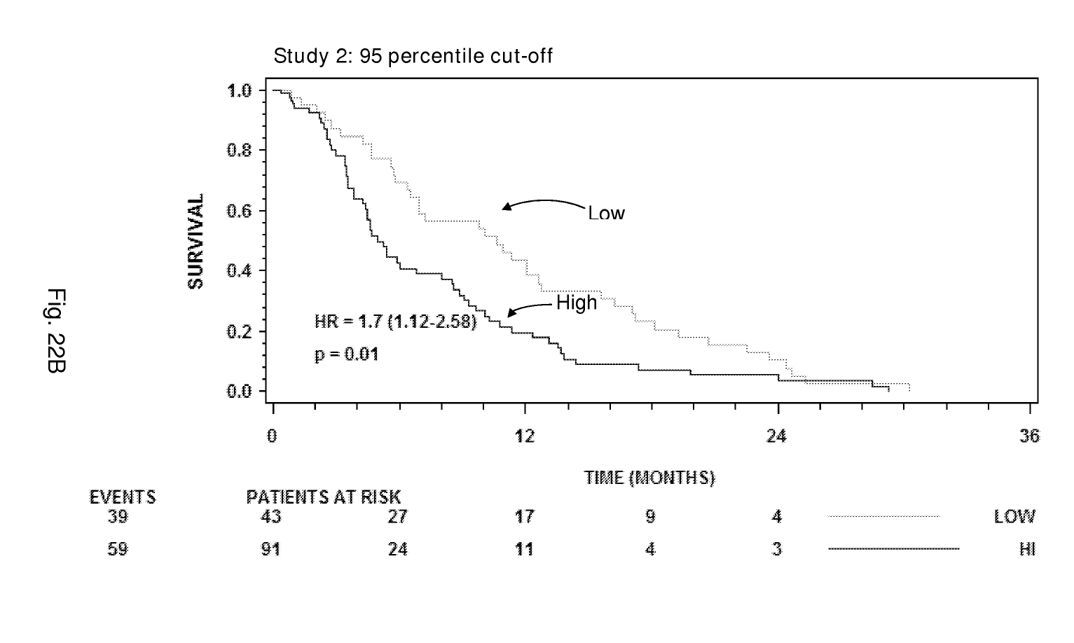

Univariate Cox analysis showed that high pretreatment plasma YKL-40 (log transformed continuous variable (base 2)), was associated with short overall survival (HR=1.23, 95% Cl: 1.09-1.39, p=0.0006), Table 8. From this analysis the 6 months survival of patients with plasma YKL-40 levels <84 μg/l (first tertile), ≧84 and ≦218 μg/l (second tertile) and >218 μg/l (third quartile) was 68%, 72%, and 46%, respectively. The Kaplan-Meier curves for these 3 groups for overall survival are illustrated in FIG. 19A. Significantly shorter survival was found for the patients with the highest plasma YKL-40 levels.

Multivariate Cox analysis (plasma YKL-40 and KRAS status) showed that pretreatment plasma YKL-40 (log transformed continuous variable (base 2): HR=1.23, 95% Cl: 1.09-1.39, p=0.0007) and KRAS status (mutated vs. wildtype: HR=1.67, 1.17-2.39, p=0.0044) were independent biomarkers of overall survival. The corresponding results when plasma YKL-40 was dichotomized according to the plasma YKL-40 level in healthy subjects (age-corrected 95% level used as cut-off) are also given in Table 8, and plasma YKL-40 remained significant (HR=1.83, 95%: 1.28-2.60, p=0.0008) and independent of KRAS. In another multivariate Cox analysis (including plasma YKL-40, KRAS, performance status, age and gender) plasma YKL-40 remained significant (HR=1.17, 95% Cl: 1.02-1.33, p=0.021).

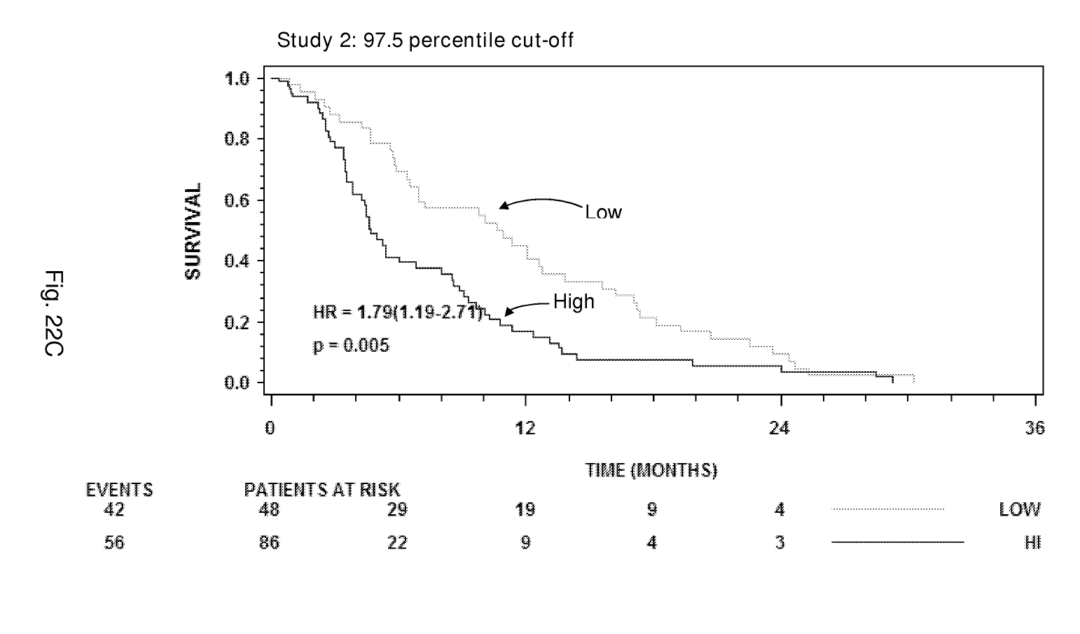

The Kaplan-Meier curves for plasma YKL-40 (the tertiles of the patients plasma YKL-40 levels are used as cut-off) and overall survival in patients with KRAS wild type are illustrated in FIG. 20A and in patients with KRAS mutations in FIG. 20B. In both patients groups significantly shorter survival were found for the patients with the highest plasma YKL-40 levels.

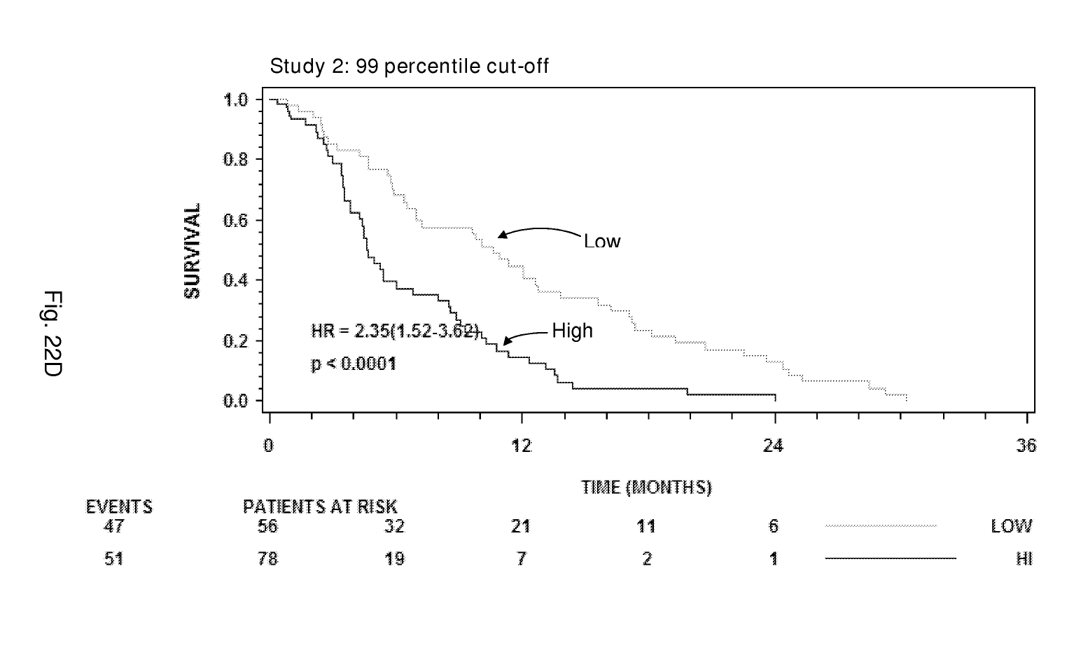

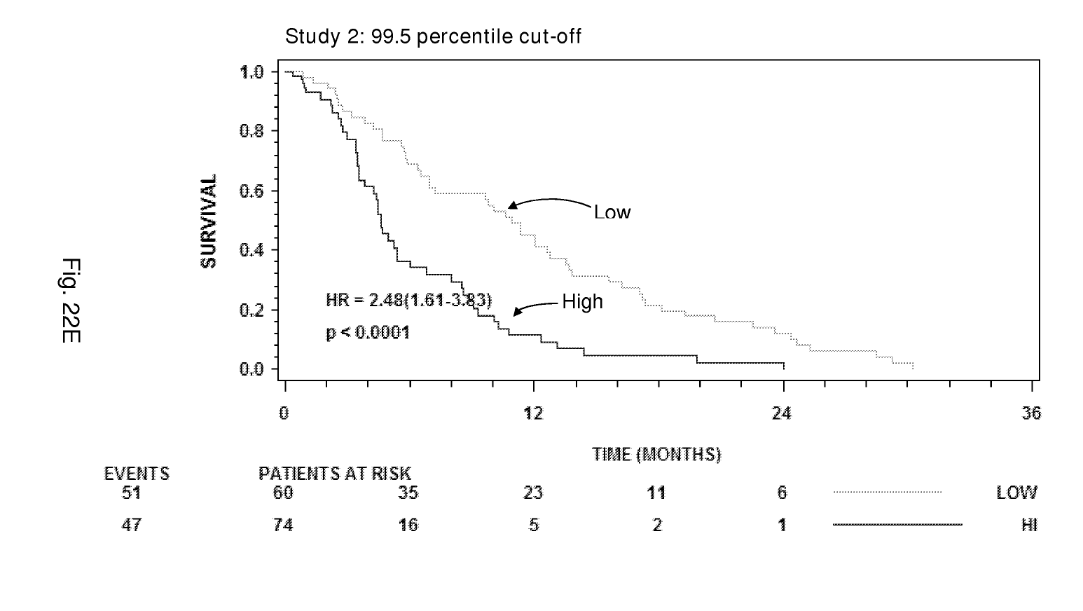

The Kaplan-Meier curves for plasma YKL-40 and overall survival in all patients included in Study 1 according to increasing cut-off levels of age-corrected plasma YKL-40 levels in healthy subjects: 90%, 95%, 97.5%, 99%, 99.5%, and 99.9% are given in FIG. 21A-F. Shorter survival was found with increasing cut-off, and the HRs increased with increasing cut-offs.

Study 2:

The median overall survival was 7.1 months. Overall survival in patients with KRAS wild type was 10.1 months compared to 6.0 months in patients with KRAS mutations (p=0.043).

Univariate Cox analysis showed that high pretreatment serum YKL-40 (log transformed continuous variable (base 2)), was associated with short overall survival (HR=1.30, 95% Cl: 1.09-1.56, p=0.003), Table 8. From this analysis the 6 months survival of patients with serum YKL-40 levels <94 μg/l (first tertile), Group 2: A4 and 253 μg/l (second tertile) and >253 μg/l (third tertile) was 67%, 53%, and 31%, respectively. The Kaplan-Meier curves for these 3 groups for overall survival are illustrated in FIG. 19B. Significantly shorter survival was found for the patients with the highest serum YKL-40 levels.

Multivariate Cox analysis (serum YKL-40 and KRAS status) showed that pretreatment serum YKL-40 (log transformed continuous variable (base 2): HR=1.41, 95% Cl: 1.18-1.69, p=0.0002) and KRAS status (mutated vs. wildtype: HR=1.57, 95% Cl: 1.02-2.42, p=0.042) were independent biomarkers of overall survival. The corresponding results when serum YKL-40 was dichotomized according to the serum YKL-40 level in healthy subjects (age-corrected 95% level used as cut-off) are also given in Table 8, and serum YKL-40 remained significant (HR=2.13, 95%: 1.40-3.33, p=0.0008) and independent of KRAS. In multivariate Cox analysis (including plasma YKL-40, KRAS, performance status) serum YKL-40 (HR=1.36, 95% Cl: 1.13-1.62, p=0.0009), KRAS (HR=1.58, 95% Cl: 1.03-2.44, p=0.037), and performance status (HR=1.69, 95%: 1.20-2.39, p=0.0028) were all significant biomarkers of survival.

The Kaplan-Meier curves for serum YKL-40 (the tertiles of the patients serum YKL-40 levels are used as cut-off) and overall survival in patients with KRAS wild type are illustrated in FIG. 20C and in patients with KRAS mutations in FIG. 20D. In both patients groups significantly shorter survival were found for the patients with the highest serum YKL-40 levels.

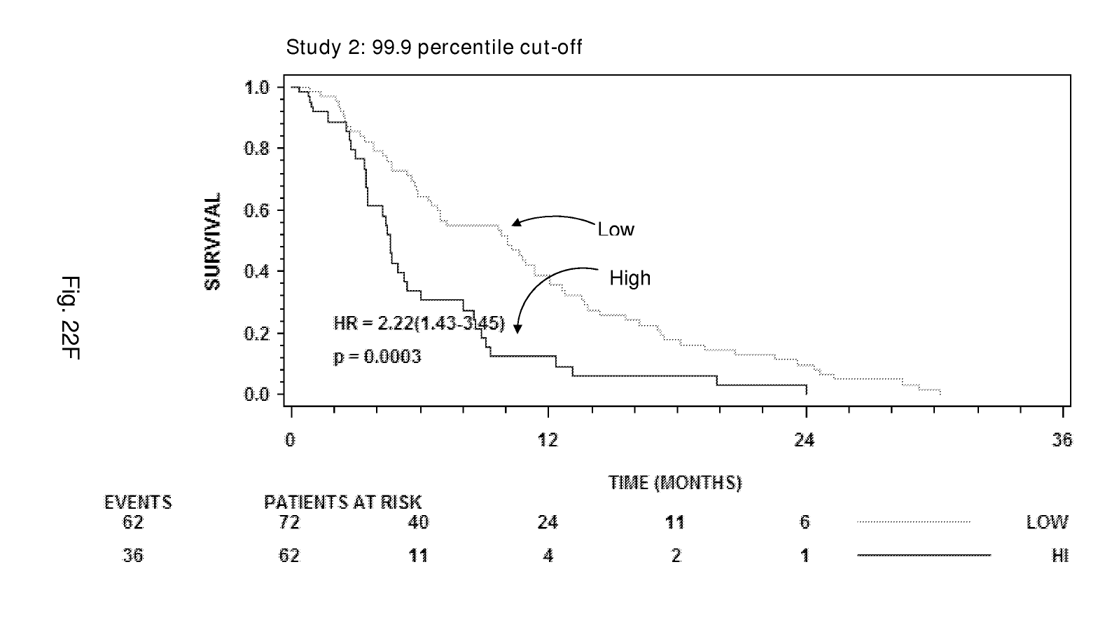

The Kaplan-Meier curves for serum YKL-40 and overall survival in all patients included in Study 2 according to increasing cut-off levels of age-corrected serum YKL-40 levels in healthy subjects: 90%, 95%, 97.5%, 99%, 99.5%, and 99.9% are given in FIG. 22A-F. Shorter survival was found with increasing cut-off, and the HRs increased with increasing cut-offs.

TABLE 8
Univariate and multivariate analyses of overall survival for the
pretreatment levels of plasma or serum YKL-40 and KRAS status in
patients with metastatic colorectal cancer treated with cetuximab.

| | Univariate analysis | | | Multivariate analysis | | |
|---|---|---|---|---|---|---|
| Variables | HR | 95% CI | P | HR | 95% CI | P |

Study 1

| | | | | | | |
|---|---|---|---|---|---|---|
| Plasma | 1.23 | 1.09-1.39 | 0.0006 | 1.23 | 1.09-1.39 | 0.0007 |
| YKL-40# | | | | | | |
| KRAS, | 1.63 | 1.16-2.30 | 0.005 | 1.67 | 1.17-2.39 | 0.0044 |
| mutations | | | | | | |
| Plasma | 1.78 | 1.26-2.53 | 0.001 | 1.83 | 1.28-2.60 | 0.0008 |
| YKL-40§ | | | | | | |
| KRAS, | 1.63 | 1.16-2.30 | 0.005 | 1.72 | 1.21-2.46 | 0.0027 |
| mutations | | | | | | |

Study 2

| | | | | | | |
|---|---|---|---|---|---|---|
| Serum | 1.30 | 1.09-1.56 | 0.003 | 1.41 | 1.18-1.69 | 0.0002 |
| YKL-40# | | | | | | |
| KRAS, | 1.55 | 1.01-2.39 | 0.045 | 1.57 | 1.02-2.42 | 0.042 |
| mutations | | | | | | |
| Serum | 1.59 | 1.04-2.45 | 0.03 | 2.13 | 1.40-3.33 | 0.0008 |
| YKL-40§ | | | | | | |
| KRAS, | 1.55 | 1.01-2.39 | 0.045 | 1.61 | 1.04-2.49 | 0.034 |
| mutations | | | | | | |

HR = Hazard ratio.
CI = Confidence interval.
#Plasma and serum YKL-40 levels are log transformed and used as a continuous variable (base 2).
The HR is for one unit on the log scale, i.e. if the HR is 1.23, this means that the hazard increases by 23% for each doubling of YKL-40.
§Plasma and serum YKL-40 levels are dichotomized (high vs. normal according to the age-corrected upper 95% percentage limit of plasma and serum YKL-40 in healthy subjects).

Conclusions

High pretreatment plasma YKL-40 and serum YKL-40 levels were prognostic biomarkers of short overall survival in two independent studies of patients with metastatic colorectal cancer treated with third-line cetuximab in combination with irinotecan. In both studies plasma YKL-40 and serum YKL-40 were independent of KRAS mutation status. In one of the studies data were available regarding response to cetuximab and progression free survival and high serum YKL-40 was associated with poor response and short progression free survival. Thus YKL-40 may be used to locate the true responders among the patients with KRAS wild type (approximately 40% of all patients with KRAS wild type). Pretreatment plasma YKL-40 and serum YKL-40 may therefore be both a new predictive biomarker of response to cetuximab and a prognostic biomarker of short survival in patients treated with cetuximab. Furthermore, by monitoring the YKL-40 level during the treatment period the progression of the disease may be monitored and the treatment be adapted accordingly.

### Example 6

Plasma and Serum YKL-40 Concentrations in Patients with Metastatic Colorectal Cancer During Treatment with Cetuximab and Irinotecan are Associated with Progression Free Survival and Overall Survival

Patients and Methods

As described for Example 5 herein.

Statistical Analysis

The analysis of updated YKL-40 levels has been done using a Cox proportional hazard model with YKL-40 as a time dependent covariate. This model includes treatment (Study 1 and Study 2) and KRAS status. Kaplan-Meier estimates of survival probabilities using a landmark at approximately 2.5 months have been done for progression free survival and overall survival.

Results

Study 1 and 2 Combined:

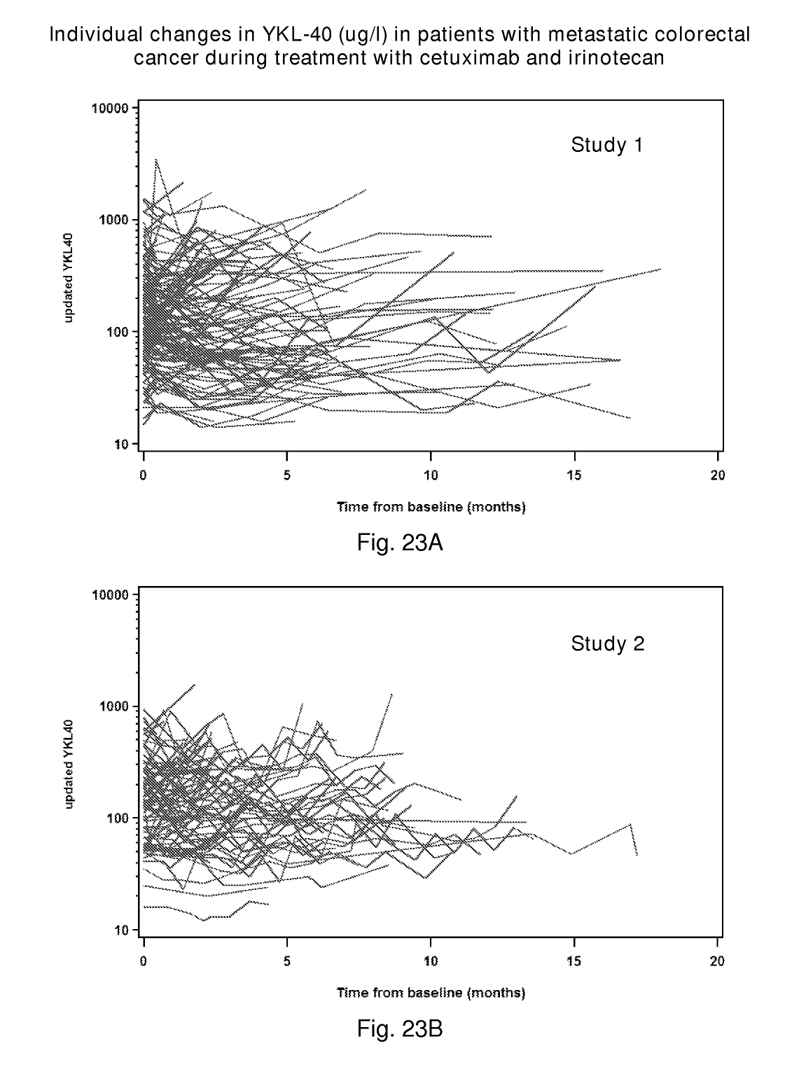

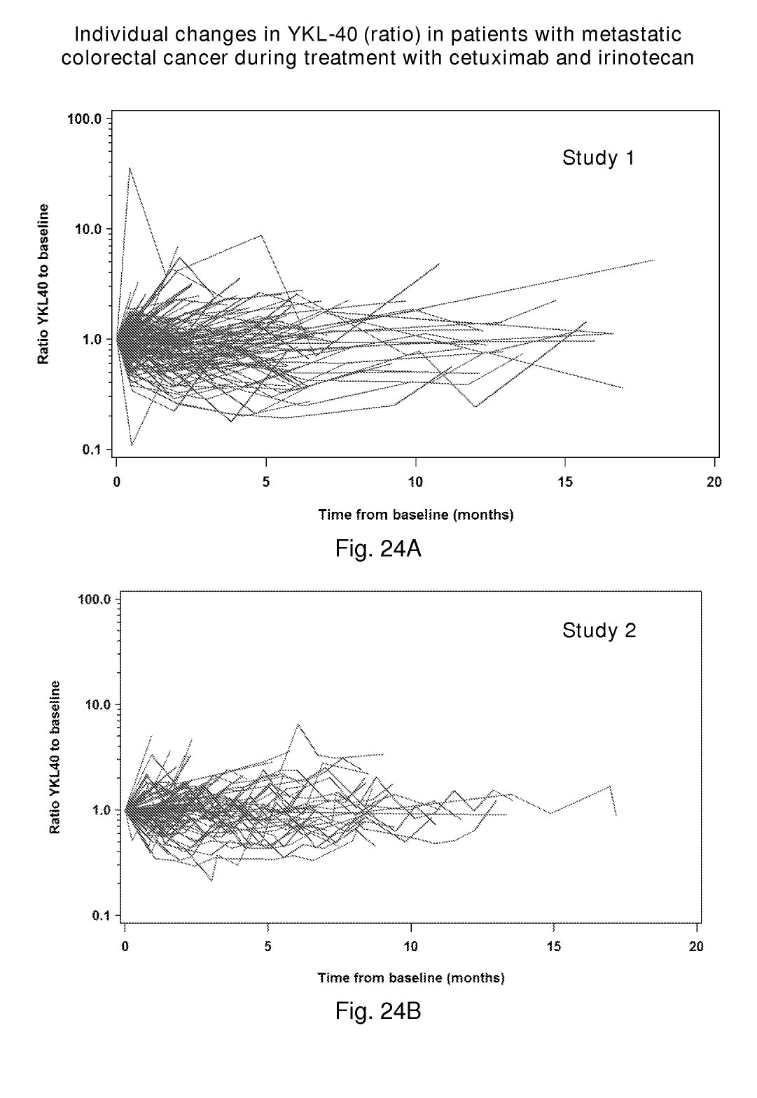

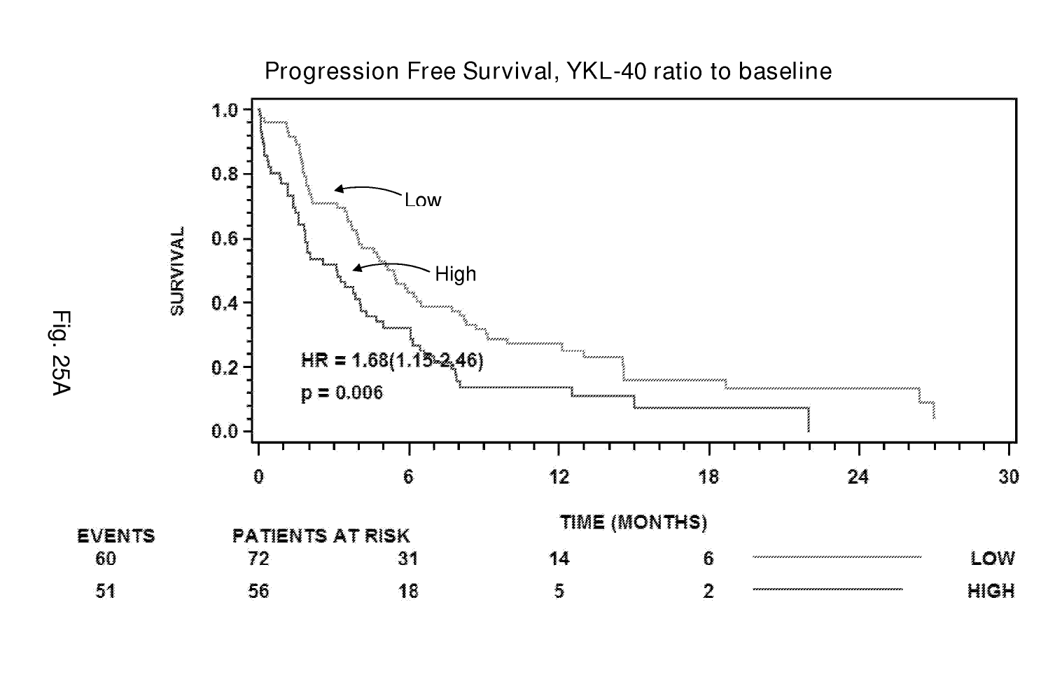

FIG. 23A (Study 1) and 23B (Study 2) illustrate the individual changes in YKL-40 (μg/l) in patients with metastatic colorectal cancer during treatment with cetuximab and irinotecan. FIG. 24A (Study 1) and 24B (Study 2) show the changes in the ratios of YKL-40 (compared to pre-treatment levels).

During treatment with cetuximab and irinotecan YKL-40 increased compared to pretreatment (baseline) levels in some patients with metastatic colorectal cancer (2 weeks mean ratio 1.21 (95% Cl: 0.81-1.60), 2 months mean ratio 1.17 (95% Cl: 1.03-1.30), 4 months mean ratio 1.04 (0.91-1.17), 6 months mean ratio 1.11 (95% Cl: 0.90-1.32), and 8 months mean ratio 1.12 (95% Cl: 0.90-1.33).

Multivariate analysis of updated YKL-40 levels showed that high YKL-40 ratio was associated with short progression free survival (HR=1.30, 95% Cl: 1.10-1.54, p=0.002) and short overall survival (HR=1.38, 95% Cl: 1.17-1.63, p=0.0002). The updated YKL-40 values (log transformed) (adjusted for Study and KRAS mutation status) were also associated with progression free survival (HR=1.11, 95% Cl: 1.04-1.20, p=0.002) and overall survival (HR=1.23, 95% Cl 1.14-1.33, p<0.0001).

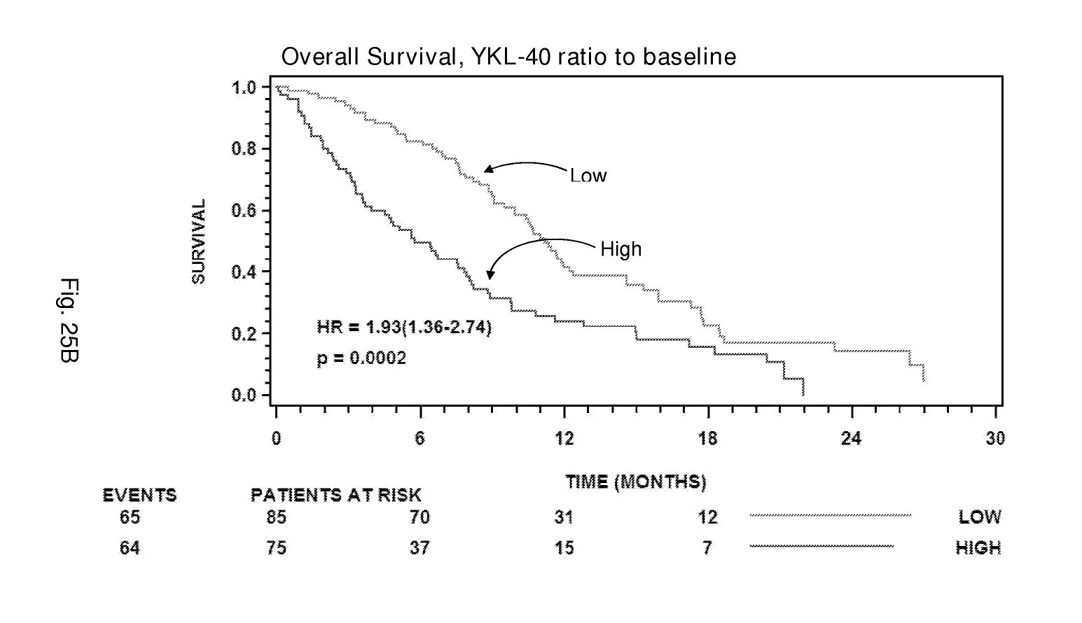

Kaplan-Meier estimates of progression free survival and overall survival and landmark time approximately 2-3 months after start of treatment with cetuximab and irinotecan are shown in FIGS. 25A and 25B. YKL-40 was dichotomized according to high or low YKL-40 ratio at this time point (defined as YKL-40 levels at 2-3 months compared to pretreatment YKL-40 levels). The 104 patients from Study 1 and 53 patients from Study 2 are combined. A high ratio is a ratio of above 1, and a low ratio is a ratio equal to/below 1, i.e. corresponding to an increase or a no-change/decrease in the YKL-40 level.

Conclusion

During treatment with cetuximab and irinotecan in patients with metastatic colorectal cancer the updated YKL-40 levels as well as the ratio of updated YKL-40 levels to the pre-treatment level were associated to progression free survival and overall survival, with high values indicating poor prognosis. These results were independent of KRAS status. These are novel observations and suggest that changes in YKL-40 during treatment with cetuximab may be a useful biomarker to monitor in patients with colorectal cancer.

### REFERENCES

Bigg H F, Wait R, Rowan A D, Cawston T E. The mammalian chitinase-like lectin, YKL-40, binds specifically to type I collagen fibril formation. J Biol Chem 2006; 281:21082-95.

Bojesen S E, Tybjærg-Hansen A, Nordestgaard B G. Integrin β3 leu33pro homozygosity and risk of cancer. J Natl Cancer Inst 2003; 95:1150-7.

Boot R G, van Achterberg T A E, van Aken B E, Renkema G H, Jacobs M J H M, Aerts J M F G, et al. Strong induction of members of the chitinase family of proteins in atherosclerosis. Chitotriosidase and human cartilage gp-39 expressed in lesion macrophages. Arterioscler Thromb Vasc Biol 1999; 19:687-94.

Bunn, et al., U.S. Pat. No. 5,213,961

Clarke R, Shipley M, Lewington S, et al. Underestimation of risk associations due to regression dilution in long-term follow-up of prospective studies. Am J Epidemiol 1999; 150:341-53.

Coligan, et al., Unit 9, Current Protocols in Immunology, Wiley Interscience, 1991).

De Ceuninck F, Gaufillier S, Bonnaud A, Sabatini M, Lesur, C, Pastoureau P. YKL-40 (Cartilage gp-39) induces proliferative events in cultured chondrocytes and synoviocytes and increases glycosaminoglycan synthesis in chondrocytes. Biochem Biophys Res Commun 2001; 285:926-31.

Harlow and Lane, Antibodies, A Laboratory Manual, Cold Spring Harbor Laboratory, 1988.

Harvey S, Weisman M, O'Dell J, et al. Chondrex: new marker of joint disease. Clin Chem 1998; 44:509-16.

Innis, et al., PCR Protocols: A Guide to Methods and Applications, Acad. Press, 1990.

Johansen J S, Williamson M K, Rice J S, Price P A. Identification of proteins secreted by human osteoblastic cells in culture. J Bone Miner Res 1992; 7:501-12.

Johansen J S, Jensen B V, Roslind A, Nielsen D, Price P A. Serum YKL-40, a new prognostic biomarker in cancer patients? Cancer Epidemiol Biomarkers Prey 2006; 15:194-202. A

Johansen J S. Studies on serum YKL-40 as a biomarker in diseases with inflammation, tissue remodelling, fibrosis and cancer. Dan Med Bull 2006; 53:172-209. B

Juel K, Helweg-Larsen K. The Danish registers of causes of death. Dan Med Bull 1999; 46:354-7.

Kirkwood T B L. Geometric means and measures of dispersion. Biometrics 1979; 35:908-9.

Kohler and Milstein, Nature, 256:495, 1975.

Kohsaka, et al., Nuc. Acids Res., 21:3469-3472, 1993

Kushner I, Rzewnicki D, Samols D. What does minor elevation of C-reactive protein signify? Am J Med 2006; 119:166.e17-166.e28.

Langone, et al. eds. Acad. Press, 1981.

Ling H, Recklies A D. The chitinase 3-like protein human cartilage glycoprotein 39 inhibits cellular responses to the inflammatory cytokines interleukin-1 and tumour necrosis factor-alpha. Biochem J 2004; 380:651-9.

Millis A J T, Hoyle M, Kent L. In vitro expression of a 38,000 dalton heparin-binding glycoprotein by morphologically differentiated smooth muscle cells. J Cell Physiol 1986; 127:366-72.

Nishikawa K C, Millis A J T. gp38k (CHI3L1) is a novel adhesion and migration factor for vascular cells. Exp Cell Res 2003; 287:79-87.

Nordestgaard B G, Benn M, Schnohr P, Tybjærg-Hansen A. Nonfasting trigycerides and risk of myocardial infarction, ischemic heart disease, and death in men and women. JAMA 2007; 298:299-308.

Ockene I S, Matthews C E, Rifai N, Ridker P M, Reed G, Stanek E. Variability and classification accuracy of serial high-sensitivity C-reactive protein measurements in healthy adults. Clin Chem 2001; 47:444-50.

Recklies A D, White C, Ling H. The chitinase 3-like protein human cartilage 39 (HC-gp39) stimulates proliferation of human connective-tissue cells and activates both extracellular signal-regulated kinase-and protein kinase B-mediated signalling pathways. Biochem J 2002; 365:119-26

Recklies A D, Ling H, White C, Bernier S M. Inflammatory cytokines induce production of CHI3L1 by articular chondrocytes. J Biol Chem. 2005; 280:41213-21.

Remington's Pharmaceutical Sciences, 16th Ed., Mack Publishing Co., Easton, Pa., 1980.

Renkema G H, Boot R G, Au F L, et al. Chitotriosidase, a chitinase, and the 39-kDa human cartilage glycoprotein, a chitin-binding lectin, are homologues of family 18 glycosyl hydrolases secreted by human macrophages. Eur J Biochem 1998; 251:504-9.

Salacinski, et al., Anal. Biochem., 117:136-146, 1981

Schnohr P, Jensen J S, Scharling H, Nordestgaard B G. Coronary heart disease risk factors ranked by importance for the individual and community. A 21 year follow-up of 12 000 men and women from The Copenhagen City Heart Study. Eur Heart J 2002; 23:620-6.

Shackelton L M, Mann D M, Millis A J T. Identification of a 38-kDa heparin-binding glycoprotein (gp38k) in differentiating vascular smooth muscle cells as a member of a group of proteins associated with tissue remodelling. J Biol Chem 1995; 270:13076-83.

1. A method for determining a therapy for a specific disease or disorder, for which the YKL-40 level may be increased, in a subject, said method comprising:
i) determining the level of YKL-40 in a sample obtained from the subject; and
ii) comparing the level of YKL-40 with one or more reference levels of YKL-40 from the following age dependent cut-off values defined as:
the 90^{th} percentile: ln(plasma YKL-40 μg/l)=3.5+0.02×age (years),
the 95^{th} percentile: ln(plasma YKL-40 μg/l )=3.6+0.02×age (years), and
the 97.5^{th} percentile: ln(plasma YKL-40 μg/l )=3.9+0.02×age (years);
wherein the level of YKL-40 with respect to the reference levels indicates the progress and/or state of said specific disease or disorder, and therefore the therapy to be initiated or continued, wherein the higher the level of YKL-40 the more severe the specific disease or disorder is classified as and the higher is the efficacy required of the therapy to be initiated, or the more must the ongoing treatment be altered or replaced for another more efficient treatment.

2-8. (canceled)

9. The method according to claim 1, wherein the one or more reference levels of YKL-40 is a set of YKL-40 age dependent cut-off values defined in the following table:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 40 | 44 | 54 | 59 | 65 |
| 30-39 | 48 | 54 | 65 | 72 | 80 |
| 40-49 | 59 | 65 | 80 | 88 | 98 |
| 50-59 | 72 | 80 | 98 | 108 | 119 |
| 60-69 | 88 | 98 | 119 | 132 | 145 |
| 70-79 | 108 | 119 | 154 | 161 | 178 |
| 80-89 | 132 | 145 | 178 | 196 | 217 |

10. The method according claim 1, wherein the one or more reference levels of YKL-40 is a set of YKL-40 age dependent reference levels obtained by measuring the YKL-40 levels in samples from age distributed subpopulations of healthy individuals, wherein the set of YKL-40 age dependent reference levels is defined in the following table:

| | Age dependent reference levels for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 33-40 | 37-44 | 45-54 | 49-59 | 55-65 |
| 30-39 | 40-48 | 45-54 | 55-65 | 60-72 | 67-80 |
| 40-49 | 49-59 | 55-65 | 67-80 | 74-88 | 81-98 |
| 50-59 | 60-72 | 67-80 | 81-98 | 90-108 | 99-119 |
| 60-69 | 74-88 | 81-98 | 99-119 | 110-132 | 122-145 |
| 70-79 | 90-108 | 99-119 | 122-154 | 134-161 | 148-178 |
| 80-89 | 110-132 | 122-145 | 148-178 | 164-196 | 181-217 |

11. (canceled)

12. The method according to claim 1, wherein the one or more reference levels is one or more previously determined levels of YKL-40 from the said same subject and wherein the one or more reference levels of YKL-40 are YKL-40 plasma levels that are age adjusted by adding 0.5 μg/l per year for women, and 0.8 μg/l per year for men.

13. (canceled)

14. The method according to claim 1, wherein a level of YKL-40 in the sample being increased to at least a factor of 1.70 or more compared to the YKL-40 reference level indicates that the specific disease or disorder has evolved to a more severe stage of the disease or disorder, more preferably increased to at least a factor of 1.75; yet even more preferably increased to at least a factor of 1.75, such as e.g. a factor of 1.80, or a factor of 1.90, or a factor of 2; most preferably increased to at least a factor of 2, such as e.g. a factor of 2.10, a factor of 2.20, a factor of 2.25, or a factor of 2.50 compared to the YKL-40 reference level indicates that the specific disease or disorder has evolved to a more severe stage or wherein a level of YKL-40 in the sample being increased by 109% compared to the YKL-40 reference level indicates that the specific disease or disorder has evolved to a more severe stage or:
wherein a level of YKL-40 in the sample being decreased at least to a factor of 0.50 compared to the YKL-40 reference level indicates that a specific disease or disorder has evolved to a less severe stage of the disease or disorder, more preferably decreased at least to a factor of 0.48, such as e.g. a factor of 0.45, a factor of 0.43, a factor of 0.40, or a factor of 0.38, compared to the YKL-40 reference level indicates that the specific disease or disorder has evolved to a less severe stage or wherein a level of YKL-40 in the sample being decreased by 52% compared to the YKL-40 reference level indicates that the specific disease or disorder has evolved to a less severe stage.

15-36. (canceled)

37. The method according to claim 1, wherein the biological sample is blood, serum, or plasma and the subject is a mammal, preferably a human.

38-48. (canceled)

49. A method for monitoring therapeutic treatment of a specific disease or disorder, for which the YKL-40 level may be increased, in a subject, said subject being treated for the specific disease, said method comprising
i) determining the level of YKL-40 in a sample obtained from the subject;
ii) comparing the level of YKL-40 with one or more reference levels of YKL-40 from the following age dependent cut-off values defined as:
the 90^{th} percentile: ln(plasma YKL-40 μg/l)=3.5+0.02×age (years),
the 95^{th} percentile: ln(plasma YKL-40 μg/l)=3.6+0.02×age (years), and
the 97.5^{th} percentile: ln(plasma YKL-40 μg/l)=3.9+0.02×age (years);
or
comparing the level of YKL-40 with one or more previously determined levels of YKL-40 from the same subject:
where a level of YKL-40 in the sample being increased to at least a factor 1.60 compared to the reference level of YKL-40 indicates that the disease or disorder has evolved to a more severe stage; and
where a level of YKL-40 in the sample being decreased to at least a factor 0.60 compared to the reference level of YKL-40 indicates that the disease or disorder has evolved to a less severe stage;
wherein the level of YKL-40 with respect to the reference levels indicates the progress and/or state of said specific disease or disorder, and therefore the degree of efficacy of the ongoing therapeutic treatment; and
iii) based thereon determining whether the therapeutic treatment of the specific disease or disorder is to be continued, terminated or replaced, wherein the higher the level of YKL-40 the more severe the specific disease or disorder is classified as, and the higher is the efficacy required of the therapy to be initiated, or the more must the ongoing treatment be altered or replaced for another more efficient treatment.

50. The method according to claim 49, wherein the determination in step i) is performed after initiation of the treatment in question.

51. The method according to claim 49, wherein the one or more reference levels of YKL-40 is a set of YKL-40 age dependent cut-off values defined in the following table:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 40 | 44 | 54 | 59 | 65 |
| 30-39 | 48 | 54 | 65 | 72 | 80 |
| 40-49 | 59 | 65 | 80 | 88 | 98 |
| 50-59 | 72 | 80 | 98 | 108 | 119 |
| 60-69 | 88 | 98 | 119 | 132 | 145 |
| 70-79 | 108 | 119 | 154 | 161 | 178 |
| 80-89 | 132 | 145 | 178 | 196 | 217 |

52. The method according to claim 49, wherein the one or more reference levels of YKL-40 is a set of YKL-40 age dependent reference levels obtained by measuring the YKL-40 levels in samples from age distributed subpopulations of healthy individuals, wherein the set of YKL-40 age dependent reference levels is defined in the following table:

| | Age dependent reference levels for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 33-40 | 37-44 | 45-54 | 49-59 | 55-65 |
| 30-39 | 40-48 | 45-54 | 55-65 | 60-72 | 67-80 |
| 40-49 | 49-59 | 55-65 | 67-80 | 74-88 | 81-98 |
| 50-59 | 60-72 | 67-80 | 81-98 | 90-108 | 99-119 |
| 60-69 | 74-88 | 81-98 | 99-119 | 110-132 | 122-145 |
| 70-79 | 90-108 | 99-119 | 122-154 | 134-161 | 148-178 |
| 80-89 | 110-132 | 122-145 | 148-178 | 164-196 | 181-217 |

53. The method according to claim 49, wherein the one or more reference levels is one or more previously determined levels of YKL-40 from the said same subject.

54. The method according to claim 49, wherein the one or more reference levels of YKL-40 are YKL-40 plasma levels that are age adjusted by adding 0.5 μg/l per year for women, and 0.8 μg/l per year for men.

55. The method according to claim 49, wherein a level of YKL-40 in the sample being increased to at least a factor of 1.70 or more compared to the YKL-40 reference level indicates that the specific disease or disorder has evolved to a more severe stage of the disease or disorder, more preferably increased to at least a factor of 1.75; yet even more preferably increased to at least a factor of 1.75, such as e.g. a factor of 1.80, or a factor of 1.90, or a factor of 2; most preferably increased to at least a factor of 2, such as e.g. a factor of 2.10, a factor of 2.20, a factor of 2.25, or a factor of 2.50 compared to the YKL-40 reference level indicates that the specific disease or disorder has evolved to a more severe stage or wherein a level of YKL-40 in the sample being increased by 109% compared to the YKL-40 reference level indicates that the specific disease or disorder has evolved to a more severe stage or
wherein a level of YKL-40 in the sample being decreased at least to a factor of 0.50 compared to the YKL-40 reference level indicates that a specific disease or disorder has evolved to a less severe stage of the disease or disorder, more preferably decreased at least to a factor of 0.48, such as e.g. a factor of 0.45, a factor of 0.43, a factor of 0.40, or a factor of 0.38, compared to the YKL-40 reference level indicates that the specific disease or disorder has evolved to a less severe stage or wherein a level of YKL-40 in the sample being decreased by 52% compared to the YKL-40 reference level indicates that the specific disease or disorder has evolved to a less severe stage.

56. A method for determining a prognosis for a subject suffering from a specific disease or disorder for which the YKL-40 level may be increased, said method comprising
i) determining the level of YKL-40 in a sample obtained from the subject;
ii) comparing said level of YKL-40 with one or more reference levels of YKL-40 from the following age dependent cut-off values defined as:
the 90^{th} percentile: ln(plasma YKL-40 μg/l)=3.5+0.02×age (years),
the 95^{th} percentile: ln(plasma YKL-40 μg/l)=3.6+0.02×age (years), and
the 97.5^{th} percentile: ln(plasma YKL-40 μg/l)=3.9+0.02×age (years);
wherein the level of YKL-40 with respect to the reference levels indicates the development or progression of said disease or disorder during or after the specific treatment regime and therefore the prognosis, wherein the higher the YKL-40 level the more severe the prognosis is.

57. The method according to claim 56, wherein the determination in step i) is performed after initiation of the treatment in question.

58. The method according to claim 56, wherein the one or more reference levels of YKL-40 is a set of YKL-40 age dependent cut-off values defined in the following table:

| | Age dependent cut-off values for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 40 | 44 | 54 | 59 | 65 |
| 30-39 | 48 | 54 | 65 | 72 | 80 |
| 40-49 | 59 | 65 | 80 | 88 | 98 |
| 50-59 | 72 | 80 | 98 | 108 | 119 |
| 60-69 | 88 | 98 | 119 | 132 | 145 |
| 70-79 | 108 | 119 | 154 | 161 | 178 |
| 80-89 | 132 | 145 | 178 | 196 | 217 |

59. The method according to claim 56, wherein the one or more reference levels of YKL-40 is a set of YKL-40 age dependent reference levels obtained by measuring the YKL-40 levels in samples from age distributed subpopulations of healthy individuals, wherein the set of YKL-40 age dependent reference levels is defined in the following table:

| | Age dependent reference levels for healthy subjects | | | | |
|---|---|---|---|---|---|
| | 70^{th} | 75^{th} | 85^{th} | 90^{th} | 95^{th} |
| Age | percentile | percentile | percentile | percentile | percentile |
| intervals | (μg/l | (μg/l | (μg/l | (μg/l | (μg/l |
| (years) | YKL-40) | YKL-40) | YKL-40) | YKL-40) | YKL-40) |
| 20-29 | 33-40 | 37-44 | 45-54 | 49-59 | 55-65 |
| 30-39 | 40-48 | 45-54 | 55-65 | 60-72 | 67-80 |
| 40-49 | 49-59 | 55-65 | 67-80 | 74-88 | 81-98 |
| 50-59 | 60-72 | 67-80 | 81-98 | 90-108 | 99-119 |
| 60-69 | 74-88 | 81-98 | 99-119 | 110-132 | 122-145 |
| 70-79 | 90-108 | 99-119 | 122-154 | 134-161 | 148-178 |
| 80-89 | 110-132 | 122-145 | 148-178 | 164-196 | 181-217 |

60. The method according to claim 56, wherein the one or more reference levels is one or more previously determined levels of YKL-40 from the said same subject.

61. The method according to claim 56, wherein the one or more reference levels of YKL-40 are YKL-40 plasma levels that are age adjusted by adding 0.5 μg/l per year for women, and 0.8 μg/l per year for men.

62. The method according to claim 56, wherein a level of YKL-40 in the sample being increased to at least a factor of 1.70 or more compared to the YKL-40 reference level indicates that the specific disease or disorder has evolved to a more severe stage of the disease or disorder, more preferably increased to at least a factor of 1.75; yet even more preferably increased to at least a factor of 1.75, such as e.g. a factor of 1.80, or a factor of 1.90, or a factor of 2; most preferably increased to at least a factor of 2, such as e.g. a factor of 2.10, a factor of 2.20, a factor of 2.25, or a factor of 2.50 compared to the YKL-40 reference level indicates that the specific disease or disorder has evolved to a more severe stage or wherein a level of YKL-40 in the sample being increased by 109% compared to the YKL-40 reference level indicates that the specific disease or disorder has evolved to a more severe stage or
wherein a level of YKL-40 in the sample being decreased at least to a factor of 0.50 compared to the YKL-40 reference level indicates that a specific disease or disorder has evolved to a less severe stage of the disease or disorder, more preferably decreased at least to a factor of 0.48, such as e.g. a factor of 0.45, a factor of 0.43, a factor of 0.40, or a factor of 0.38, compared to the YKL-40 reference level indicates that the specific disease or disorder has evolved to a less severe stage or wherein a level of YKL-40 in the sample being decreased by 52% compared to the YKL-40 reference level indicates that the specific disease or disorder has evolved to a less severe stage.
